(19) United States
(12) Patent Application Publication
(10) Pub. No.: US 2025/0057898 A1
(43) Pub. Date: Feb. 20, 2025

(54) USE OF LACTIC ACID BACTERIA TO INHIBIT METHANOGEN GROWTH OR REDUCE METHANE EMISSIONS

(71) Applicant: FONTERRA CO-OPERATIVE GROUP LIMITED, Auckland (NZ)

(72) Inventors: Graeme Trevor Attwood, Hamilton (NZ); Laureen Crouzet, Hamilton (NZ); Shalome Anitta Bassett, Auckland (NZ); James William Dekker, Auckland (NZ); Jeremy Paul Hill, Auckland (NZ)

(21) Appl. No.: 18/723,377

(22) PCT Filed: Dec. 22, 2022
(86) PCT No.: PCT/IB2022/062650

§ 371 Date: Jun. 21, 2024

(30) Foreign Application Priority Data
Dec. 23, 2021 (AU) 2021904257
Dec. 23, 2021 (AU) 2021904258

(51) Int. Cl. A61K 35/747; A23K 10/18; A23K 50/10; C12N 1/20; C12R 1/225

(52) U.S. Cl. CPC A61K 35/747; A23K 10/18; A23K 50/10; C12N 1/205; A23V 2400/175; C12R 2001/225

(57) ABSTRACT

This invention relates to the use of a strain of lactic acid bacteria for improving the body weight or body composition of animals, such as ruminant and/or monogastric animals, improving feed efficiency, growth, productivity, and/or milk or meat yield, and/or for inhibiting the growth of methane-producing bacteria and/or archaea in the digestive tract of animals, reducing the ability of the rumen and/or gastrointestinal microbiome to produce methane, reducing methane emissions by an animal, delivering a microorganism to an animal, and/or reducing the greenhouse gas emission footprint of an animal. Animal feed compositions are also provided.

### TECHNICAL FIELD

This invention relates to the use of a strain of lactic acid bacteria for improving the body weight or body composition of animals, such as ruminant and/or monogastric animals, improving feed efficiency, growth, productivity, and/or milk or meat yield, and/or for inhibiting the growth of methane-producing bacteria and/or archaea in the digestive tract of animals, reducing the ability of the rumen and/or gastrointestinal microbiome to produce methane, reducing methane emissions by an animal, delivering a microorganism to an animal, and/or reducing the greenhouse gas emission footprint of an animal. Animal feed compositions are also provided.

### BACKGROUND

The use of lactic acid bacteria (LAB) as probiotics to attempt to improve animal health and nutrition is well documented, and have been investigated as alternatives to antibiotic used as growth promoters.

On-farm, LAB are used as direct-fed microbials (DFMs), probiotics and as silage inoculants. Their actions are exerted in strain- and host-specific manners. Studies have reported benefits that include a reduction in the incidence of diarrhoea, promotion of ruminal development, improved feed efficiency, increased body weight gain, and reduction in morbidity (Krehbiel et al., 2003).

The use of LAB to reduce methane emissions from ruminants has also been proposed.

A major source of methane emissions is the fermentation of organic matter by methanogenic bacteria and archaea. One prevalent source of anthropogenic methane emissions is in agriculture, where methane is produced by enteric fermentation in the digestive tract of ruminants, and from manure such as pig manure. These sources accounted for ˜30% of total global anthropogenic methane emissions in 2017 (Jackson et al., 2020), and methane emissions from pigs account for ˜10% of the total methane production from livestock in China (Mi et al., 2019).

In addition, not only does methanogenesis in ruminants and monogastric animals result in greenhouse gas emissions, but it is also energetically wasteful to the animal. It has long been recognised that methane production dramatically impacts the efficiency with which animals convert feed into metabolic energy. This decrease in efficiency results because methane represents a caloric loss to a ruminant of approximately 5-10% of its total caloric intake, and represents ˜0.1-3.3% of digestive energy loss in pigs (Mi et al., 2019). To date however, research on the potential use of LAB to reduce methane emissions has been limited.

Thus, there remains a need for methods and compositions useful for increasing feed efficiency, increasing body weight or improving body composition of animals, enhancing growth and/or productivity, and/or increasing milk or meat production in animals. Methods and compositions for inhibiting the growth of methane-producing bacteria and/or archaea in the gastrointestinal tract of animals, reducing the ability of the rumen and/or gastrointestinal microbiome to produce methane, reducing methane emissions by animals, delivering a microorganism to animals, and/or reducing the greenhouse gas emission footprint of animals are also desirable.

It is an object of this invention to go some way towards achieving one or more of these desiderata, or at least to offer the public a useful choice.

### SUMMARY OF THE INVENTION

In a first aspect, the invention provides isolated Lacticaseibacillus rhamnosus strain FNZ129, NMIA accession number V21/015446 dated 2 Aug. 2021, or a derivative thereof.

In one embodiment, the Lacticaseibacillus rhamnosus strain FNZ129 is a biologically pure culture.

In a second aspect, the invention provides a food or feed composition comprising Lacticaseibacillus rhamnosus strain FNZ129, NMIA accession number V21/015446 dated 2 Aug. 2021, or a derivative thereof.

In a third aspect, the invention provides a feed composition for:
a) improving the body weight and/or body composition of an animal,
b) increasing feed efficiency in an animal,
c) enhancing the growth and/or productivity in an animal,
d) increasing the yield of milk and/or milk components produced from an animal,
e) inhibiting the growth of methane-producing bacteria and/or archaea in the gastrointestinal tract of an animal,
f) reducing the ability of the gastrointestinal microbiome to produce methane,
g) reducing methane emissions by an animal,
h) delivering a microorganism to an animal, and/or
i) reducing the greenhouse gas emission footprint of an animal, the feed composition comprising Lacticaseibacillus rhamnosus (Lactobacillus rhamnosus) strain FNZ129, NMIA accession number V21/015446 dated 2 Aug. 2021, or a derivative thereof.

In a fourth aspect, the invention provides a method for improving the body weight and/or body composition of an animal, said method comprising the step of administering to said animal a food or feed composition of the second aspect, or a feed composition of the third aspect.

In a fifth aspect, the invention provides a method for increasing feed efficiency in an animal, said method comprising the step of administering to said animal a food or feed composition of the second aspect, or a feed composition of the third aspect.

In a sixth aspect, the invention provides a method for enhancing the growth and/or productivity in an animal, said method comprising the step of administering to said animal a food or feed composition of the second aspect, or a feed composition of the third aspect.

In a seventh aspect, the invention provides a method for increasing the yield of milk and/or milk components produced from an animal, said method comprising the step of administering to said animal a food or feed composition of the second aspect, or a feed composition of the third aspect.

In an eighth aspect, the invention provides a method for inhibiting the growth of methane-producing bacteria and/or archaea in the gastrointestinal tract of an animal, said method comprising the step of administering to said animal a food or feed composition of the second aspect, or a feed composition of the third aspect.

In a ninth aspect, the invention provides a method for reducing the ability of the gastrointestinal microbiome of an animal to produce methane, said method comprising the step of administering to said animal a food or feed composition of the second aspect, or a feed composition of the third aspect.

In a tenth aspect, the invention provides a method for reducing methane emissions by an animal, said method comprising the step of administering to said animal a food or feed composition of the second aspect, or a feed composition of the third aspect.

In an eleventh aspect, the invention provides a method for delivering a microorganism to an animal, said method comprising the step of administering to said animal a food or feed composition of the second aspect, or a feed composition of the third aspect.

In a twelfth aspect, the invention provides a method for reducing the greenhouse gas emission footprint of an animal, said method comprising the step of administering to said animal a food or feed composition of the second aspect, or a feed composition of the third aspect.

In a thirteenth aspect, the invention provides a method for improving the body weight and/or body composition of an animal, said method comprising the step of administering to said animal an effective amount of Lacticaseibacillus rhamnosus (Lactobacillus rhamnosus) strain FNZ129, NMIA accession number V21/015446 dated 2 Aug. 2021, or a derivative thereof.

In a fourteenth aspect, the invention provides a method for increasing feed efficiency in an animal, wherein the method comprises administering to the animal an effective amount of Lacticaseibacillus rhamnosus (Lactobacillus rhamnosus) strain FNZ129, NMIA accession number V21/015446 dated 2 Aug. 2021, or a derivative thereof.

In a fifteenth aspect, the invention provides a method for enhancing the growth and/or productivity in an animal, said method comprising the step of administering to said animal an effective amount of Lacticaseibacillus rhamnosus (Lactobacillus rhamnosus) strain FNZ129, NMIA accession number V21/015446 dated 2 Aug. 2021, or a derivative thereof.

In a sixteenth aspect, the invention provides a method for increasing the yield of milk and/or milk components produced from an animal, said method comprising the step of administering to said animal an effective amount of Lacticaseibacillus rhamnosus (Lactobacillus rhamnosus) strain FNZ129, NMIA accession number V21/015446 dated 2 Aug. 2021, or a derivative thereof.

In a seventeenth aspect, the invention provides a method for inhibiting the growth of methane-producing bacteria and/or archaea in the gastrointestinal tract of an animal, wherein the method comprises administering to the animal an effective amount of Lacticaseibacillus rhamnosus (Lactobacillus rhamnosus) strain FNZ129, NMIA accession number V21/015446 dated 2 Aug. 2021, or a derivative thereof.

In a eighteenth aspect, the invention provides a method for reducing the ability of the gastrointestinal microbiome to produce methane, wherein the method comprises administering to an animal an effective amount of Lacticaseibacillus rhamnosus (Lactobacillus rhamnosus) strain FNZ129, NMIA accession number V21/015446 dated 2 Aug. 2021, or a derivative thereof.

In a ninteenth aspect, the invention provides a method for reducing methane emissions by an animal, wherein the method comprises administering to the animal an effective amount of Lacticaseibacillus rhamnosus (Lactobacillus rhamnosus) strain FNZ129, NMIA accession number V21/015446 dated 2 Aug. 2021, or a derivative thereof.

In a twentieth aspect, the invention provides a method for delivering a microorganism to an animal, wherein the method comprises administering to the animal an effective amount of Lacticaseibacillus rhamnosus (Lactobacillus rhamnosus) strain FNZ129, NMIA accession number V21/015446 dated 2 Aug. 2021, or a derivative thereof.

In a twenty-first aspect, the invention provides a method for reducing the greenhouse gas emission footprint of an animal, wherein the method comprises administering to the animal an effective amount of Lacticaseibacillus rhamnosus (Lactobacillus rhamnosus) strain FNZ129, NMIA accession number V21/015446 dated 2 Aug. 2021, or a derivative thereof.

In a twenty-second aspect, the invention provides an animal to which the method of any one of the fourth to twenty-first aspects has been applied.

In a twenty-third aspect, the invention provides a method for producing an animal product, for example a dairy, meat, or wool product, having a reduced greenhouse gas emission footprint, the method comprising:
a. providing the animal of the twenty-second aspect, and
b. producing an animal product from the animal.

In a twenty-fourth aspect, the invention provides use of Lacticaseibacillus rhamnosus (Lactobacillus rhamnosus) strain FNZ129, NMIA accession number V21/015446 dated 2 Aug. 2021, or a derivative thereof, for the manufacture of a composition for improving the body weight and/or body composition of an animal, increasing feed efficiency in an animal, enhancing the growth and/or productivity in an animal, increasing the yield of milk and/or milk components produced from an animal, inhibiting the growth of methane-producing bacteria and/or archaea in the gastrointestinal tract of an animal, reducing the ability of the gastrointestinal microbiome to produce methane, reducing methane emissions by an animal, delivering a microorganism to an animal, and/or reducing the greenhouse gas emission footprint of an animal.

In a twenty-fifth aspect, the invention provides Lacticaseibacillus rhamnosus (Lactobacillus rhamnosus) strain FNZ129, NMIA accession number V21/015446 dated 2 Aug. 2021, or a derivative thereof, for use in improving the body weight and/or body composition of an animal, increasing feed efficiency in an animal, enhancing the growth and/or productivity in an animal, increasing the yield of milk and/or milk components produced from an animal, inhibiting the growth of methane-producing bacteria and/or archaea in the gastrointestinal tract of an animal, reducing the ability of the gastrointestinal microbiome to produce methane, reducing methane emissions by an animal, delivering a microorganism to an animal, and/or reducing the greenhouse gas emission footprint of an animal.

In a twenty-sixth aspect, the invention provides a ruminant feed composition for inhibiting the growth of methane-producing bacteria and/or archaea in the forestomach of ruminant animals, reducing the ability of the rumen microbiome to produce methane, reducing methane emissions by a ruminant animal, increasing feed efficiency in a ruminant animal, enhancing the growth and/or productivity in a ruminant animal, increasing the yield of milk and/or milk components produced from a ruminant animal, or improving the body weight and/or body composition of a ruminant animal, the feed composition comprising Lacticaseibacillus rhamnosus strain FNZ129, NMIA accession number V21/015446 dated 2 Aug. 2021, or a derivative thereof.

In a twenty-seventh aspect, the invention provides a method for inhibiting the growth of methane-producing bacteria and/or archaea in the forestomach of a ruminant animal, said method comprising the step of administering to said animal a food or feed composition according to the second aspect, or a ruminant feed composition according to the twentieth aspect.

In a twenty-eighth aspect, the invention provides a method for reducing the ability of the rumen microbiome of a ruminant animal to produce methane, said method comprising the step of administering to said animal a food or feed composition according to the second aspect, or a ruminant feed composition according to the twentieth aspect.

In a twenty-ninth aspect, the invention provides a method for reducing methane emissions by a ruminant animal, said method comprising the step of administering to said animal a food or feed composition according to the second aspect, or a ruminant feed composition according to the twentieth aspect.

In a thirtieth aspect, the invention provides a method for increasing feed efficiency in a ruminant animal, said method comprising the step of administering to said animal a food or feed composition according to the second aspect, or a ruminant feed composition according to the twentieth aspect.

In a thirty-first aspect, the invention provides a method for enhancing the growth and/or productivity in a ruminant animal, said method comprising the step of administering to said animal a food or feed composition according to the second aspect, or a ruminant feed composition according to the twentieth aspect.

In a thirty-second aspect, the invention provides a method for increasing the yield of milk and/or milk components produced from a ruminant animal, said method comprising the step of administering to said animal a food or feed composition according to the second aspect, or a ruminant feed composition according to the twentieth aspect.

In a thirty-third aspect, the invention provides a method for improving the body weight or body composition of a ruminant animal, said method comprising the step of administering to said animal a food or feed composition according to the second aspect, or a ruminant feed composition according to the twentieth aspect.

In a thirty-fourth aspect, the invention provides a method for inhibiting the growth of methane-producing bacteria and/or archaea in the forestomach of ruminant animals, wherein the method comprises administering to a ruminant animal an effective amount of Lacticaseibacillus rhamnosus strain FNZ129, NMIA accession number V21/015446 dated 2 Aug. 2021, or a derivative thereof.

In a thirty-fifth aspect, the invention provides a method for reducing methane emissions by a ruminant animal, wherein the method comprises administering to the animal an effective amount of Lacticaseibacillus rhamnosus strain FNZ129, NMIA accession number V21/015446 dated 2 Aug. 2021, or a derivative thereof.

In a thirty-sixth aspect, the invention provides a method for increasing feed efficiency in a ruminant animal, wherein the method comprises administering to the animal an effective amount of Lacticaseibacillus rhamnosus strain FNZ129, NMIA accession number V21/015446 dated 2 Aug. 2021, or a derivative thereof.

In a thirty-seventh aspect, the invention provides a method for enhancing the growth and/or productivity in a ruminant animal, said method comprising the step of administering to said animal an effective amount of Lacticaseibacillus rhamnosus strain FNZ129, NMIA accession number V21/015446 dated 2 Aug. 2021, or a derivative thereof.

In a thirty-eighth aspect, the invention provides a method for increasing the yield of milk and/or milk components produced from a ruminant animal, said method comprising the step of administering to said animal an effective amount of Lacticaseibacillus rhamnosus strain FNZ129, NMIA accession number V21/015446 dated 2 Aug. 2021, or a derivative thereof.

In a thirty-ninth aspect, the invention provides a method for improving the body weight and/or body composition of a ruminant animal, said method comprising the step of administering to said animal an effective amount of Lacticaseibacillus rhamnosus strain FNZ129, NMIA accession number V21/015446 dated 2 Aug. 2021, or a derivative thereof.

In a fortieth aspect, the invention provides a method for reducing the ability of the rumen microbiome to produce methane, wherein the method comprises administering to the animal an effective amount of Lacticaseibacillus rhamnosus strain FNZ129, NMIA accession number V21/015446 dated 2 Aug. 2021, or a derivative thereof.

In a forty-first aspect, the invention provides a method for improving the absorptive capacity of the forestomach, for example increasing the absorptive capacity for volatile fatty acids (VFAs), wherein the method comprises administering to the animal an effective amount of Lacticaseibacillus rhamnosus strain FNZ129, NMIA accession number V21/015446 dated 2 Aug. 2021, or a derivative thereof.

In a forty-second aspect, the invention provides a method for enhancing the physical and/or functional development of the rumen, or other chambers of the forestomach, in a ruminant, for example a young ruminant, for example a young ruminant prior to weaning, wherein the method comprises administering to the animal an effective amount of Lacticaseibacillus rhamnosus strain FNZ129, NMIA accession number V21/015446 dated 2 Aug. 2021, or a derivative thereof.

In a forty-third aspect, the invention provides a use of Lacticaseibacillus rhamnosus strain FNZ129, NMIA accession number V21/015446 dated 2 Aug. 2021, or a derivative thereof, for the manufacture of a composition for inhibiting the growth of methane-producing bacteria and/or archaea in the forestomach of ruminant animals, reducing the ability of the rumen microbiome to produce methane, reducing methane emissions by a ruminant animal, increasing feed efficiency in a ruminant animal, increasing the yield of milk and/or milk components produced from a ruminant animal, or improving the body weight and/or body composition of a ruminant animal.

In a forty-fourth aspect, the invention provides Lacticaseibacillus rhamnosus strain FNZ129, NMIA accession number V21/015446 dated 2 Aug. 2021, or a derivative thereof, for use in inhibiting the growth of methane-producing bacteria and/or archaea in the forestomach of ruminant animals, reducing the ability of the rumen microbiome to produce methane, reducing methane emissions by a ruminant animal, increasing feed efficiency in a ruminant animal, increasing the yield of milk and/or milk components produced from a ruminant animal, or improving the body weight and/or body composition of a ruminant animal.

In a forty-fifth aspect, the invention provides monogastric feed composition for inhibiting the growth of methane-producing bacteria and/or archaea in the gastrointestinal tract of monogastric animals, reducing the ability of the gastrointestinal microbiome to produce methane, reducing methane emissions by a monogastric animal, increasing feed efficiency in a monogastric animal, enhancing the growth and/or productivity in a monogastric animal, increasing the yield of milk and/or milk components produced from a monogastric animal, or improving the body weight and/or body composition of a monogastric animal, the feed composition comprising Lacticaseibacillus rhamnosus strain FNZ129, NMIA accession number V21/015446 dated 2 Aug. 2021, or a derivative thereof.

In a further aspect, the invention provides a method for inhibiting the growth of methane-producing bacteria and/or archaea in the gastrointestinal tract of monogastric animals, said method comprising the step of administering to said animal a food or feed composition according to the second aspect, or a monogastric feed composition according to the forty-fifth aspect.

In a further aspect, the invention provides a method for reducing the ability of the gastrointestinal microbiome to produce methane, said method comprising the step of administering to said animal a food or feed composition according to the second aspect, or a monogastric feed composition according to the forty-fifth aspect.

In a further aspect, the invention provides a method for reducing methane emissions by a monogastric animal, said method comprising the step of administering to said animal a food or feed composition according to the second aspect, or a monogastric feed composition according to the forty-fifth aspect.

In a further aspect, the invention provides a method for increasing feed efficiency in a monogastric animal, said method comprising the step of administering to said animal a food or feed composition according to the second aspect, or a monogastric feed composition according to the forty-fifth aspect.

In a further aspect, the invention provides a method for enhancing the growth and/or productivity in a monogastric animal, said method comprising the step of administering to said animal a food or feed composition according to the second aspect, or a monogastric feed composition according to the forty-fifth aspect.

In a further aspect, the invention provides a method for increasing the yield of milk and/or milk components produced from a monogastric animal, said method comprising the step of administering to said animal a food or feed composition according to the second aspect, or a monogastric feed composition according to the forty-fifth aspect.

In a further aspect, the invention provides a method for improving the body weight or body composition of a monogastric animal, said method comprising the step of administering to said animal a food or feed composition according to the second aspect, or a monogastric feed composition according to the forty-fifth aspect.

In a further aspect the invention provides a method for inhibiting the growth of methane-producing bacteria and/or archaea in the gastrointestinal tract of monogastric animals, wherein the method comprises administering to a monogastric animal an effective amount of Lacticaseibacillus rhamnosus strain FNZ129, NMIA accession number V21/015446 dated 2 Aug. 2021, or a derivative thereof.

In a further aspect the invention provides a method for reducing methane emissions by a monogastric animal, wherein the method comprises administering to the animal an effective amount of Lacticaseibacillus rhamnosus strain FNZ129, NMIA accession number V21/015446 dated 2 Aug. 2021, or a derivative thereof.

In a further aspect the invention provides a method for increasing feed efficiency in a monogastric animal, wherein the method comprises administering to the animal an effective amount of Lacticaseibacillus rhamnosus strain FNZ129, NMIA accession number V21/015446 dated 2 Aug. 2021, or a derivative thereof.

In a further aspect, the invention provides a method for reducing the ability of the gastrointestinal microbiome to produce methane, wherein the method comprises administering to the animal an effective amount of Lacticaseibacillus rhamnosus strain FNZ129, NMIA accession number V21/015446 dated 2 Aug. 2021, or a derivative thereof.

In a further aspect, the invention provides a method for enhancing the growth and/or productivity in a monogastric animal, wherein the method comprises administering to a monogastric animal an effective amount of Lacticaseibacillus rhamnosus strain FNZ129, NMIA accession number V21/015446 dated 2 Aug. 2021, or a derivative thereof.

In a further aspect, the invention provides a method for improving the body weight and/or body composition of monogastric animal, wherein the method comprises administering to a monogastric animal an effective amount of Lacticaseibacillus rhamnosus strain FNZ129, NMIA accession number V21/015446 dated 2 Aug. 2021, or a derivative thereof.

In a further aspect, the invention provides a method for improving the absorptive capacity of the gastrointestinal tract, for example increasing the absorptive capacity for volatile fatty acids (VFAs), wherein the method comprises administering to the animal an effective amount of Lacticaseibacillus rhamnosus strain FNZ129, NMIA accession number V21/015446 dated 2 Aug. 2021, or a derivative thereof.

In a further aspect, the invention provides a method for enhancing the physical and/or functional development of the gastrointestinal tract, functional achievement of the gastrointestinal tract, or promoting maturation of the gastrointestinal tract, in a monogastric animal, for example a young animal, for example a young animal prior to weaning, wherein the method comprises administering to the animal an effective amount of Lacticaseibacillus rhamnosus strain FNZ129, NMIA accession number V21/015446 dated 2 Aug. 2021, or a derivative thereof.

In a further aspect, the invention provides use of Lacticaseibacillus rhamnosus strain FNZ129, NMIA accession number V21/015446 dated 2 Aug. 2021, or a derivative thereof for the manufacture of a composition for inhibiting the growth of methane-producing bacteria and/or archaea in the gastrointestinal tract of monogastric animals, reducing the ability of the gastrointestinal microbiome to produce methane, reducing methane production by a monogastric animal, increasing feed efficiency in a monogastric animal, or improving the body weight and/or body composition of a monogastric animal.

In a further aspect, the invention provides Lacticaseibacillus rhamnosus strain FNZ129, NMIA accession number V21/015446 dated 2 Aug. 2021, or a derivative thereof for use in inhibiting the growth of methane-producing bacteria and/or archaea in the gastrointestinal tract of monogastric animals, reducing the ability of the gastrointestinal microbiome to produce methane, reducing methane production, by a monogastric animal, increasing feed efficiency in a monogastric animal, or improving the body weight and/or body composition of a monogastric animal.

In a further aspect, the invention provides a method for delivering a microorganism to an animal, said method comprising the step of administering to said animal a food or feed composition according to the second aspect, or a feed composition according to the third aspect.

In a further aspect, the invention provides a method for delivering a microorganism to an animal, said method comprising the step of administering to said animal an effective amount of Lacticaseibacillus rhamnosus strain FNZ129, NMIA accession number V21/015446 dated 2 Aug. 2021, or a derivative thereof.

The following embodiments and preferences may relate alone or in any combination of any two or more to any of the above aspects.

In one embodiment, the isolated Lacticaseibacillus rhamnosus strain FNZ129 is a biologically pure culture.

In some embodiments, the food or feed composition is a ruminant feed composition. In some embodiments, the feed composition is a ruminant feed composition, and the animal is a ruminant animal. In some embodiments, the food or feed composition is a feed composition for a monogastric animal. In some embodiments, the feed composition is a monogastric feed composition, and the animal is a monogastric animal.

In some embodiments, the feed composition is a fermented yoghurt-style composition, and wherein the fermented yoghurt-style composition is formed through a process of growing L. rhamnosus FNZ129 using a milk-based carrier or non-milk-based carrier.

In some embodiments, the feed composition is or comprises Partial or Total Mixed Ration (TMR), corn, soybean, forage, grain, distiller's grain, sprouted grain, legumes, fibre, fodder, grass, hay, straw, silage, kernel, leaves, meal, mash feed, fruit pulp, vegetable pulp, fruit or vegetable pomace, citrus meal, wheat shorts, corn cob meal, lick block, or molasses.

In some embodiments, the feed composition further comprises at least one microorganism of a different species or strain, a vaccine that inhibits methanogens or methanogenesis, and/or a natural or chemically-synthesised inhibitor of methanogenesis and/or methanogen inhibitor such as bromoform.

In some embodiments, the feed composition further comprises one or more agents selected from one or more prebiotics, one or more probiotics, one or more postbiotics, one or more sources of dietary fibre, one or more galactooligosaccharides, one or more short chain galactooligosaccharides, one or more long chain galactooligosaccharides, one or more fructooligosaccharides, inulin, one or more galactans, one or more fructans, lactulose, one or more milk-derived oligosaccharides (for example, 2′-fucosyllactose, 3′-fucosyllactose, 3′-sialyllactose, 6′-sialyllactose, lacto-N-tetraose, lacto-N-neotetraose), or any mixture of any two or more thereof.

In some embodiments, the derivative of the L. rhamnosus FNZ129 is a cell lysate of the L. rhamnosus FNZ129, a cell suspension of the L. rhamnosus FNZ129, a metabolite of the L. rhamnosus FNZ129, or a culture supernatant of the L. rhamnosus FNZ129, or killed L. rhamnosus FNZ129.

In some embodiments, the ruminant feed composition is a bovine feed composition. In some embodiments, the ruminant feed composition is a goat feed composition. In some embodiments, the ruminant feed composition is a sheep feed composition. In some embodiments, the ruminant feed composition is a bison feed composition. In some embodiments, the ruminant feed composition is a yak feed composition. In some embodiments, the ruminant feed composition is a water buffalo feed composition. In some embodiments, the ruminant feed composition is a deer feed composition. In some embodiments, the ruminant feed composition is a camel feed composition. In some embodiments, the ruminant feed composition is an alpaca feed composition. In some embodiments, the ruminant feed composition is a llama feed composition. In some embodiments, the ruminant feed composition is a wildebeest feed composition. In some embodiments, the ruminant feed composition is an antelope feed composition. In some embodiments, the ruminant feed composition is a nilgai feed composition.

In some embodiments, the L. rhamnosus FNZ129 or derivative thereof is administered in a feed composition. In some embodiments the feed composition is a livestock feed composition. In some embodiments the feed composition is a companion animal feed composition. In some embodiments the feed composition is a pet food composition. In some embodiments the feed composition is a herbivore feed composition. In some embodiments the feed composition is an omnivore feed composition. In some embodiments the feed composition is a carnivore feed composition. In some embodiments the feed composition is a poultry feed composition. In some embodiments the feed composition is a cat feed composition. In some embodiments the feed composition is a dog feed composition. In some embodiments the feed composition is a pig feed composition. In some embodiments the feed composition is a horse feed composition. In some embodiments the feed composition is a donkey feed composition. In some embodiments the feed composition is a rabbit feed composition. In some embodiments the feed composition is a chicken feed composition. In some embodiments the feed composition is a duck feed composition. In some embodiments the feed composition is a goose feed composition. In some embodiments the feed composition is a turkey feed composition. In some embodiments the feed composition is a human food composition.

In some embodiments, the animal is a ruminant animal. In some embodiments, the animal is a monogastric animal.

In one embodiment, the method enhances anatomical development of the rumen. For example, the method enhances development of rumen epithelium and/or muscularisation, for example increasing growth of rumen mass, growth of rumen papillae, increase in papillae density, for example dorsal papillae density, and/or total surface area of the ruminal wall in the animal.

In one embodiment, the method enhances rumen weight, ruminal wall thickness, or density of rumen papillae per cm² of ruminal wall, for example compared to an untreated animal.

In one embodiment, the method increases rumen papillae length, width, and/or surface area. For example, in some embodiments, the method increases rumen papillae length, width, and/or surface area to at least 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.22, 1.24, 1.26, 1.28, 1.30, 1.32, 1.34, 1.36, 1.38, or 1.40 times that of an untreated animal.

In one embodiment, the method enhances functional achievement of the rumen, or promotes maturation of the forestomach. For example, the method stimulates rumination, enhances dry matter intake (DMI), enhances absorptive ability and/or promotes maturation towards a mature physiology.

In some embodiments, the method inhibits the growth of a methylotrophic methanogen in the forestomach and/or caecum of the animal. In some embodiments, the method inhibits the growth of a methanogen from the genus Methanosphaera in the forestomach and/or caecum of the animal. In some embodiments, the method inhibits the growth of a methylotrophic methanogen in the gastrointestinal of the animal. In one embodiment, the method inhibits the growth of a methanogen from the genus Methanosphaera in the gastrointestinal tract of the animal. In some embodiments, the method inhibits the growth of a methylotrophic methanogen in the caecum or colorectum of the animal. In one embodiment, the method inhibits the growth of a methanogen from the genus Methanosphaera in the caecum or colorectum of the animal.

In some embodiments, the L. rhamnosus FNZ129 or derivative thereof is administered in a composition that is a food, drink, food additive, drink additive, animal feed, animal feed additive, animal feed supplement, dietary supplement, carrier, vitamin or mineral premix, nutritional product, enteral feeding product, soluble, slurry, supplement, pharmaceutical, lick block, drench, tablet, capsule, pellet, bolus, or intra-ruminal product, or the L. rhamnosus FNZ129 is encapsulated, for example in liposomes, microbubbles, microparticles or microcapsules.

In a further aspect, the invention provides a composition comprising Lacticaseibacillus rhamnosus strain FNZ129, NMIA accession number V21/015446 dated 2 Aug. 2021, or a derivative thereof. In some embodiments the composition is a food, drink, food additive, drink additive, animal feed, animal feed additive, animal feed supplement, dietary supplement, carrier, vitamin or mineral premix, nutritional product, enteral feeding product, soluble, slurry, supplement, pharmaceutical, lick block, drench, tablet, capsule, pellet, bolus, or intra-ruminal product, or the L. rhamnosus FNZ129 is encapsulated, for example in liposomes, microbubbles, microparticles or microcapsules.

In some embodiments, the L. rhamnosus FNZ129 or derivative thereof is administered in drinking water, milk, milk powder, milk replacement, milk fortifier, whey, whey powder, Partial or Total Mixed Ration (TMR), a feed pellet, corn, soybean, forage, grain, distiller's grain, sprouted grain, legumes, vitamins, amino acids, minerals, fibre, fodder, grass, hay, straw, silage, kernel, leaves, meal, solubles, slurries, supplements, mash feed, meal, fruit pulp, vegetable pulp, fruit or vegetable pomace, citrus meal, wheat shorts, corn cob meal, molasses, sucrose, maltodextrin, rice hulls, vermiculite, zeolites or crushed limestone.

In some embodiments, the method comprises administering to the animal the L. rhamnosus FNZ118 in an amount of at least about 10⁴ colony forming units per kilogram of dry weight carrier feed, such as at least about 10⁵, at least about 10⁶, at least about 10⁷, at least about 10⁸, at least about 10⁹, at least about 10¹⁰, at least about 10¹¹, at least about 10¹², or at least about 10¹³ colony forming units per kilogram of dry weight carrier feed. In some embodiments, the method comprises administering to the animal the L. rhamnosus FNZ129 in an amount of from 10⁴ to 10¹³ colony forming units per kilogram of dry weight carrier feed. In one embodiment, the method comprises administering to the animal the L. rhamnosus FNZ129 in an amount from 10⁸ to 10¹² colony forming units per kilogram of dry weight carrier feed.

In some embodiments, the method comprises administering to the animal L. rhamnosus FNZ129 in an amount of from 10⁴ to 10¹⁰ colony forming units per kilogram of body weight of the animal per day.

In some embodiments, the method comprises administering to the animal L. rhamnosus FNZ129 in an amount from 10⁴ to 10¹³ colony forming units per day. In some embodiments, the method comprises administering to the animal L. rhamnosus FNZ129 in an amount from 10⁸ to 10¹² colony forming units per day.

In some embodiments, the method comprises further administering at least one microorganism of a different species or strain, a vaccine that inhibits methanogens or methanogenesis, and/or a natural or chemically-synthesised inhibitor of methanogenesis and/or methanogen inhibitor. An example of a useful inhibitor of methanogenesis is bromoform, which works by inhibiting the efficiency of the methyltransferase enzyme by reacting with the reduced vitamin B12 cofactor required for the penultimate step of methanogenesis.

In one embodiment, the method comprises further administering at least one microorganism of a different species or strain, a vaccine that inhibits methanogens or methanogenesis, and/or a natural or chemically-synthesised inhibitor of methanogenesis and/or methanogen inhibitor that targets a hydrogenotrophic methanogen, for example, a methanogen from the genus Methanobrevibacter.

In some embodiments, the L. rhamnosus FNZ129 or derivative thereof is administered separately, simultaneously or sequentially with one or more agents selected from one or more prebiotics, one or more probiotics, one or more postbiotics, one or more sources of dietary fibre, one or more galactooligosaccharides, one or more short chain galactooligosaccharides, one or more long chain galactooligosaccharides, one or more fructooligosaccharides, inulin, one or more galactans, one or more fructans, lactulose, or any mixture of any two or more thereof.

In some embodiments, the method additionally enhances the growth or productivity of the animal, for example the method increases the yield of milk and/or milk components produced from the animal. In some embodiments, the method increases the yield of milk fat, milk protein or milk solids in the milk produced from the animal.

In some embodiments, the method additionally increases the body weight and/or improves body composition, such as altering the muscle to fat ratio, of the animal. In some embodiments, the method increases the body weight and/or improves body composition, such as altering the muscle to fat ratio, of the monogastric animal. In some embodiments, the method reduces the body mass index (BMI) and/or increases the muscle to fat ratio of the monogastric animal.

In some embodiments, the ruminant animal is a bovine, goat, sheep, bison, yak, water buffalo, deer, camel, alpaca, llama, wildebeest, antelope, or nilgai. In one embodiment, the ruminant animal are cattle or sheep. In one embodiment, the ruminant animal are cattle. In one embodiment, the ruminant animal is a lactating animal. In an alternative embodiment, the ruminant animal is a pre-weaning animal, such as a calf or a lamb.

In some embodiments, the method additionally increases wool growth of the ruminant animal.

In some embodiments, the monogastric animal is a human, pig, cat, dog, horse, donkey, rabbit, or poultry. In some embodiments, the monogastric animal is a companion animal. In some embodiments, the monogastric animal is a non-human animal. In some embodiments, the monogastric animal is a pig. In some embodiments, the monogastric animal is a chicken, duck, goose or turkey.

In some embodiments, the animal is a pre-weaning animal, for example a calf, a lamb, a piglet, or a foal.

In some embodiments, the L. rhamnosus FNZ129 is administered to the animal both prior to weaning and after weaning.

In some embodiments, the administering is to a pre-weaning animal and wherein the inhibition of the growth of methane-producing bacteria and/or archaea in the gastrointestinal tract of the animal, the reduction of methane emissions, for example methane production, by the animal, and/or the increased feed efficiency in the animal persists post-weaning.

In some embodiments, the inhibition of the growth of methane-producing bacteria and/or archaea in the gastrointestinal tract of the animal, the reduction of methane emissions, for example methane production, by the animal, and/or the increased feed efficiency in the animal persists for at least 2 days, 3 days, 5 days, 1 week, 2 weeks, 1 month, 2 months, 3 months, 4 months, 5 months, 6 months, 7 months 8 months, 9 months, 10 months, 11 months, 1 year, 2 years, 3 years, 4 years, 5 years, 6 years, or 7 years after the last administration of L. rhamnosus FNZ129.

In some embodiments, the inhibition of the growth of methane-producing bacteria and/or archaea in the gastrointestinal tract of the animal, the reduction of methane emissions, for example methane production, by the animal, and/or the increased feed efficiency in the animal persists for the life of the animal.

In some embodiments, the composition is or comprises a food or feed composition according to the second aspect, or a feed composition according to the third aspect.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### BRIEF DESCRIPTION OF THE DRAWINGS

FIG. 1 shows the effect of feed supplementation with L. rhamnosus FNZ129 on the growth of pigs.

FIG. 2 shows the effect of L. rhamnosus FNZ129 on the most probable number (MPN) of methanogenic bacteria per gram of pig faeces in the caecum. L. rhamnosus significantly reduced the MPN, p=0.015.

FIG. 3 shows the effect of L. rhamnosus FNZ129 on the most probable number (MPN) of methanogenic bacteria per gram of pig faeces in the colorectum.

FIG. 4 shows the absolute concentration of the main volatile fatty acids (mM) measured in pig caecal samples collected after euthanasia. n=8 animals for control and n=7 animals for FNZ129 treatment. * T test FNZ129 vs control p<0.05.

FIG. 5 shows the absolute concentration of the main volatile fatty acids (mM) measured in pig colorectal samples collected after euthanasia. n=8 animals for control and n=7 animals for FNZ129 treatment.

### DETAILED DESCRIPTION OF THE INVENTION

The present invention is based on the finding that Lacticaseibacillus rhamnosus strain FNZ129 and derivatives thereof inhibit or suppress the growth of methane-producing bacteria and/or archaea in the gastrointestinal tract of animals and/or reduces the ability of the gastrointestinal microbiome to produce methane. For example, L. rhamnosus FNZ129 and derivatives thereof inhibit or suppress the growth of methane-producing bacteria and/or archaea in the forestomach of ruminant animals and/or reduce the ability of the rumen microbiota to produce methane. L. rhamnosus FNZ129 and derivatives thereof also inhibit the growth of methane-producing bacteria and/or archaea in the gastrointestinal tract of monogastric animals and/or reduces the ability of the gastrointestinal microbiome to produce methane. Inhibiting the growth of methane-producing bacteria and/or archaea can reduce methane emissions and may alter the volatile fatty acids (VFA) profile, total VFA concentration, residual feed intake (RFI) and/or rate of fermentation in the gastrointestinal tract, which can act as an increased energy source driving enhanced growth or increased productivity, such as milk, meat, or wool production, and can stimulate rumen and/or gastrointestinal development, such as rumen papillae development.

Accordingly, in a first aspect, the invention provides isolated Lacticaseibacillus rhamnosus strain FNZ129, NMIA accession number V21/015446 dated 2 Aug. 2021, or a derivative thereof.

In a second aspect, the invention provides a food or feed composition comprising Lacticaseibacillus rhamnosus strain FNZ129, NMIA accession number V21/015446 dated 2 Aug. 2021, or a derivative thereof.

In a third aspect, the invention provides a feed composition for:
a) improving the body weight and/or body composition of an animal,
b) increasing feed efficiency in an animal,
c) enhancing the growth and/or productivity in an animal,
d) increasing the yield of milk and/or milk components produced from an animal,
e) inhibiting the growth of methane-producing bacteria and/or archaea in the gastrointestinal tract of an animal,
f) reducing the ability of the gastrointestinal microbiome to produce methane,
g) reducing methane emissions by an animal,
h) delivering a microorganism to an animal, and/or
i) reducing the greenhouse gas emission footprint of an animal, the feed composition comprising Lacticaseibacillus rhamnosus (Lactobacillus rhamnosus) strain FNZ129, NMIA accession number V21/015446 dated 2 Aug. 2021, or a derivative thereof.

In a further aspect, the invention provides a method for improving the body weight and/or body composition of an animal, said method comprising the step of administering to said animal a food or feed composition of the second aspect, or a feed composition of the third aspect.

In a further aspect, the invention provides a method for increasing feed efficiency in an animal, said method comprising the step of administering to said animal a food or feed composition of the second aspect, or a feed composition of the third aspect.

In a further aspect, the invention provides a method for enhancing the growth and/or productivity in an animal, said method comprising the step of administering to said animal a food or feed composition of the second aspect, or a feed composition of the third aspect.

In a further aspect, the invention provides a method for increasing the yield of milk and/or milk components produced from an animal, said method comprising the step of administering to said animal a food or feed composition of the second aspect, or a feed composition of the third aspect.

In a further aspect, the invention provides a method for inhibiting the growth of methane-producing bacteria and/or archaea in the gastrointestinal tract of an animal, said method comprising the step of administering to said animal a food or feed composition of the second aspect, or a feed composition of the third aspect.

In a further aspect, the invention provides a method for reducing the ability of the gastrointestinal microbiome of an animal to produce methane, said method comprising the step of administering to said animal a food or feed composition of the second aspect, or a feed composition of the third aspect.

In a further aspect, the invention provides a method for reducing methane emissions by an animal, said method comprising the step of administering to said animal a food or feed composition of the second aspect, or a feed composition of the third aspect.

In a further aspect, the invention provides a method for delivering a microorganism to an animal, said method comprising the step of administering to said animal a food or feed composition of the second aspect, or a feed composition of the third aspect.

In a further aspect, the invention provides a method for reducing the greenhouse gas emission footprint of an animal, said method comprising the step of administering to said animal a food or feed composition of the second aspect, or a feed composition of the third aspect.

In a further aspect, the invention provides a method for improving the body weight and/or body composition of an animal, said method comprising the step of administering to said animal an effective amount of Lacticaseibacillus rhamnosus (Lactobacillus rhamnosus) strain FNZ129, NMIA accession number V21/015446 dated 2 Aug. 2021, or a derivative thereof.

In a further aspect, the invention provides a method for increasing feed efficiency in an animal, wherein the method comprises administering to the animal an effective amount of Lacticaseibacillus rhamnosus (Lactobacillus rhamnosus) strain FNZ129, NMIA accession number V21/015446 dated 2 Aug. 2021, or a derivative thereof.

In a further aspect, the invention provides a method for enhancing the growth and/or productivity in an animal, said method comprising the step of administering to said animal an effective amount of Lacticaseibacillus rhamnosus (Lactobacillus rhamnosus) strain FNZ129, NMIA accession number V21/015446 dated 2 Aug. 2021, or a derivative thereof.

In a further aspect, the invention provides a method for increasing the yield of milk and/or milk components produced from an animal, said method comprising the step of administering to said animal an effective amount of Lacticaseibacillus rhamnosus (Lactobacillus rhamnosus) strain FNZ129, NMIA accession number V21/015446 dated 2 Aug. 2021, or a derivative thereof.

In a further aspect, the invention provides a method for inhibiting the growth of methane-producing bacteria and/or archaea in the gastrointestinal tract of an animal, wherein the method comprises administering to the animal an effective amount of Lacticaseibacillus rhamnosus (Lactobacillus rhamnosus) strain FNZ129, NMIA accession number V21/015446 dated 2 Aug. 2021, or a derivative thereof.

In a further aspect, the invention provides a method for reducing the ability of the gastrointestinal microbiome to produce methane, wherein the method comprises administering to an animal an effective amount of Lacticaseibacillus rhamnosus (Lactobacillus rhamnosus) strain FNZ129, NMIA accession number V21/015446 dated 2 Aug. 2021, or a derivative thereof.

In a further aspect, the invention provides a method for reducing methane emissions by an animal, wherein the method comprises administering to the animal an effective amount of Lacticaseibacillus rhamnosus (Lactobacillus rhamnosus) strain FNZ129, NMIA accession number V21/015446 dated 2 Aug. 2021, or a derivative thereof.

In a further aspect, the invention provides a method for delivering a microorganism to an animal, wherein the method comprises administering to the animal an effective amount of Lacticaseibacillus rhamnosus (Lactobacillus rhamnosus) strain FNZ129, NMIA accession number V21/015446 dated 2 Aug. 2021, or a derivative thereof.

In a further aspect, the invention provides a method for reducing the greenhouse gas emission footprint of an animal, wherein the method comprises administering to the animal an effective amount of Lacticaseibacillus rhamnosus (Lactobacillus rhamnosus) strain FNZ129, NMIA accession number V21/015446 dated 2 Aug. 2021, or a derivative thereof.

In a further aspect, the invention provides use of Lacticaseibacillus rhamnosus (Lactobacillus rhamnosus) strain FNZ129, NMIA accession number V21/015446 dated 2 Aug. 2021, or a derivative thereof, for the manufacture of a composition for improving the body weight and/or body composition of an animal, increasing feed efficiency in an animal, enhancing the growth and/or productivity in an animal, increasing the yield of milk and/or milk components produced from an animal, inhibiting the growth of methane-producing bacteria and/or archaea in the gastrointestinal tract of an animal, reducing the ability of the gastrointestinal microbiome to produce methane, reducing methane emissions by an animal, delivering a microorganism to an animal, and/or reducing the greenhouse gas emission footprint of an animal.

In a further aspect, the invention provides Lacticaseibacillus rhamnosus (Lactobacillus rhamnosus) strain FNZ129, NMIA accession number V21/015446 dated 2 Aug. 2021, or a derivative thereof, for use in improving the body weight and/or body composition of an animal, increasing feed efficiency in an animal, enhancing the growth and/or productivity in an animal, increasing the yield of milk and/or milk components produced from an animal, inhibiting the growth of methane-producing bacteria and/or archaea in the gastrointestinal tract of an animal, reducing the ability of the gastrointestinal microbiome to produce methane, reducing methane emissions by an animal, delivering a microorganism to an animal, and/or reducing the greenhouse gas emission footprint of an animal.

In one embodiment, the methods and compositions enhance anatomical development of the rumen. For example, the method enhances development of rumen epithelium and/or muscularisation, for example increasing growth of rumen mass, growth of rumen papillae, increase in papillae density, for example dorsal papillae density, and/or total surface area of the ruminal wall in the animal.

In one embodiment, the methods and compositions disclosed herein enhance rumen weight, ruminal wall thickness, or density of rumen papillae per cm² of ruminal wall.

In one embodiment, the methods and compositions disclosed herein enhance functional achievement of the rumen. For example, the method stimulates rumination and/or enhances dry matter intake (DMI). In one embodiment the methods and compositions disclosed herein increase ruminal turnover rate and/or increase post-ruminal digestion. Without wishing to be bound by theory, it has been hypothesised that a higher rumen turnover rate selects for microorganisms that are capable of fast, heterofermentative growth on soluble sugars, producing less hydrogen, which leads to less methane formation. For example, Kamke et al (2016) note that lactate conversion to butyrate, instead of to propionate, produces 2 mol of hydrogen per hexose, which could produce 0.5 mol of methane via the hydrogenotrophic pathway, and postulate that direct fermentation of hexoses to butyrate and acetate by, for example, members of the Ruminococcaceae would produce 2.66 mol of hydrogen and allow 0.66 mol of methane to be formed. Thus, lower hydrogen production via the lactate to butyrate pathway is predicted to decrease methane production.

The term “administering” refers to the action of introducing an effective amount of L. rhamnosus strain FNZ129 or a derivative thereof into the forestomach of a ruminant animal. More particularly, this administration is an administration by oral route. This administration can in particular be carried out by supplementing the feed or drink intended for the animal with the strain; the supplemented feed or drink then being ingested by the animal.

The term “effective amount” refers to a quantity of L. rhamnosus strain FNZ129 or a derivative thereof sufficient to allow a desired effect, i.e., inhibition of the growth of methane-producing bacteria and/or archaea in the gastrointestinal tract of the animal (for example, the forestomach of the animal), a reduction in methane emissions by the animal, or an increase in feed efficiency in the animal, in comparison with a reference. The desired effect (such as inhibition of growth of methane-producing bacteria and/or archaea and/or reduction of methane production or emission) can be measured in vitro or in vivo. For example, the desired effect can be measured in vitro using the methods described herein, for example, in the Examples below, or in an artificial rumen system, such as that described in T. Hano (1993) J. Gen. Appl. Microbiol., 39, 35-45, or by in vivo oral administration to animals.

This effective amount can be administered to the animal in one or more doses.

The terms “reducing methane production” and “reducing methane emissions”, e.g., “reducing methane production by the animal” and “reducing methane emissions by the animal” refers to reducing methane production or emissions by any mechanism, and from any animal-related source. For example, when referring to a ruminant animal, the term may refer to a reduction in methane produced within the forestomach of ruminant animals, or it may refer to a reduction in methane produced or emitted by the faeces of a ruminant animal. When referring to a monogastric animal, the term may refer to a reduction in methane produced within the gastrointestinal tract of the monogastric animal, or it may refer to a reduction in methane produced or emitted by the faeces or manure of a monogastric animal.

It is anticipated that the reduction in methane production may be due to a variety of mechanisms. These may include, for example, killing methanogens (i.e. a bactericidal/archaeacidal effect), inhibiting the growth of methanogens (i.e. a bacteriostatic/archaeostatic effect), and/or inhibiting the ability of the gastrointestinal, forestomach, or rumen microbiota to produce methane. Inhibiting the ability of the gastrointestinal, forestomach, or rumen microbiota to produce methane may be via a variety of mechanisms, including, for example, physical and/or chemical changes to the gastrointestinal tract, caecal, forestomach, or rumen environment, changes to the microbiota, the inhibition of one or more methanogenic pathways, and/or cross-feeding (or disrupting cross-feeding) of intermediaries between members of the microbiome.

It will be appreciated that a reduction in greenhouse gas (GHG) emissions, such as methane emissions, is desirable. GHG emissions may be reduced either directly, for example by reducing the ability of the gastrointestinal microbiome to produce methane and/or by reducing methane emissions by an animal, or indirectly. One example of indirect reduction in GHG emissions is by altered land use and/or land retirement. Animals with improved feed efficiency (such as animals to which the methods or compositions of the present invention have been applied) may require less pasture for forage and/or less imported feed. Alternatively or additionally, more animals may be able to be farmed on a given land area, allowing the same production with reduced land use. In either case, decreased land requirements may allow unused pasture to be retired, for example by planting trees or other vegetation for carbon sequestration. Such land use changes may further lower the GHG emissions per farm, resulting in a lower GHG emission footprint per animal, and/or per kg animal product (such as a dairy, meat, or wool product).

The GHG emission footprint of an animal and/or animal product may be determined using techniques known in the art. It will be appreciated that certain GHGs produce more global warming potential than others. For example, 1 kg of methane emissions produces a global warming impact approximately equivalent to 25 kg of CO₂. To account for this, GHG emissions are typically reported as CO₂ equivalents (CO₂ₑ), i.e. the amount of CO₂ that would have an equivalent global warming impact. The GHG emission footprint may be calculated per animal, or per amount of animal product (for example, per kg milk solids, per kg meat, or per kg wool). As described above, the GHG emission footprint should take into account alterations to land use, such as planting trees or other vegetation for carbon sequestration.

The term “animal product” refers to any product produced from or by an animal, or containing any animal-derived component(s). The term is intended to include products that are directly produced by an animal (for example, milk, meat, and wool) and products that include or are made from animal ingredients, that have optionally undergone further processing, optionally with other ingredients. For example, the term is intended to include foods and beverages that contain animal ingredients, such as various dairy products (including buttermilk, cheese, cream, formula, ice cream, milk, milk powder, puddings, shakes, smoothies, and yoghurts), meat products (such as a chops, ground meat, hamburger, sausages, sausage meat, steaks, and wings) and other products that contain animal ingredients.

The term “feed efficiency” refers to the relationship between feed intake and muscle weight gain or milk yield. Microbial fermentation in the gastrointestinal tract, forestomach, or rumen produce volatile fatty acids (VFA) such as acetic acid, propionic acid and butyric acid. These fatty acids are absorbed directly from the gastrointestinal (GI) tract and/or rumen wall and used as raw materials for growth and development of the animal, milk components, and other final digested products. The majority of the energy consumed by body tissues is used to produce milk or milk components, or muscle. Thus, when the utilisation of energy is improved, milk production, e.g., milk yield, and/or milkfat, milk protein, and/or milk solids can be increased. Increases in muscle, and/or improvements in body composition, such as altered muscle/fat ratio in an animal, can also be achieved.

Feed efficiency can be calculated by dividing the weight of milk produced by an animal, or the liveweight of an animal, by the weight of dry matter consumed by that animal. Thus, an animal with a higher feed efficiency will produce more milk, milk with a higher content of milk components such as, but not limited to, fat and protein, and/or will show increased weight gain compared to an animal with a lower feed efficiency when given the same nutrient input. Feed efficiency can be measured by differences in the growth of an animal by any of the following parameters: average daily weight gain, total weight gain, feed conversion, which includes both feed:gain and gain:feed, feed efficiency, mortality, and feed intake. That is to say, improved feed efficiency can mean that the ratio of feed intake/muscle weight gain is decreased. Improved feed efficiency can also mean that the ratio of muscle weight gain/feed intake is increased. The term feed efficiency may also refer to the feed intake/weight gain or weight gain/feed intake. The feed efficiency may be standardised to account for differences in protein and fat content by using the energy-corrected milk (ECM) yield instead of the weight of milk. This can be calculated using the following formula (Tyrrell and Reid, 1965):

ECM=(12.82×weight of fat in pounds)+(7.13×weight of protein in pounds)+(0.323×weight of milk in pounds).

“Feed conversion” and “residual feed intake (RFI)” are also commonly used measures of feed efficiency, and the terms are often used almost interchangeably. In animal husbandry, feed conversion ratio or feed conversion rate is a ratio or rate measuring of the efficiency with which the bodies of livestock convert animal feed into the desired output. RFI is defined as the difference between the actual dry matter intake (DMI) of an animal and the expected DMI required for maintenance and growth.

The primary advantage of improving feed efficiency (i.e., improving the feed conversion ratio or lowering RFI) is to reduce DMI in animals without compromising growth performance, because feed-related costs often represent the largest production expense in beef or milk production. Any reduction in DMI to produce a unit of beef or milk product would minimise feed costs, resulting in maximising the overall profitability of the beef or dairy industry.

In one embodiment, the feed efficiency in a ruminant animal is increased to at least about 1.01× of the feed efficiency of an untreated animal, such as at least about 1.02×, 1.03×, 1.04×, 1.05×, 1.06×, 1.07×, 1.08×, 1.09×, 1.10×, 1.12×, 1.14×, 1.16×, 1.18×, such as at least about 1.20×.

Increased feed efficiency may result from alteration of the volatile fatty acid (VFA) profile, total VFA concentration and/or the rate of fermentation in the rumen and forestomach.

In some embodiments, L. rhamnosus strain FNZ129 or a derivative thereof promotes propionic acid production. Propionic acid has higher ATP production efficiency compared with other volatile fatty acids, and hence, feed efficiency is improved owing to the promotion of propionic acid production. Propionic acid is also glucogenic and can thus promote lactose synthesis in the mammary gland.

In some embodiments, L. rhamnosus FNZ129 or a derivative thereof shifts hydrogen metabolism from methanogenesis to short chain/volatile fatty acid (VFA) production, for example to propionic acid production. Propionate is predominantly used as a glucose precursor, and more propionate formation would likely result in a more efficient utilisation of feed energy. Maximizing the flow of metabolic hydrogen in the forestomach or rumen away from methane and toward VFA (mainly propionate) would increase the efficiency of animal farming (e.g. ruminant production) and decrease its environmental impact, and would enhance rumen development and/or rumen papillae development.

Acetate is the primary substrate for mammary lipid synthesis, along with β-hydroxybutyrate which is produced during the absorption of butyrate. Consequently, a high acetate fermentation pattern will provide substrate to maintain or increase milk fat.

Thus, in some embodiments, L. rhamnosus strain FNZ129 or a derivative thereof results in an increase in milkfat, milk protein, overall milk volume and/or milk solids as a result of increased VFAs in the forestomach or rumen, which can act as an increased energy source driving increased production.

In some embodiments, the yield of milk and/or or milk components produced from the animal are preferably increased by 1.5% or more, more preferably, by 3.0% or more, by 4.5% or more, or by 6.0% or more.

In some embodiments, L. rhamnosus strain FNZ129 or a derivative thereof results in an increase in liveweight, muscle mass, and/or fat deposition, and/or improvements in body composition, such as altered muscle/fat ratio in an animal as a result of increased VFAs in the forestomach or rumen, which can act as an increased energy source driving increased production.

In some embodiments, the liveweight of the animal is preferably increased by 1% or more, more preferably, by 2% or more, by 3% or more, by 4% or more, by 5% or more, by 6% or more, by 7% or more, by 8% or more, by 9% or more, or by 10% or more, in comparison to a reference animal.

It is anticipated that the present invention could also be used to extend the lactation cycle of a lactating ruminant, such as a cow. A cow directs a significant portion of its energy towards producing milk during lactation. After a long period of lactation, its body condition will be poorer for it. Because of this, the lactation period is usually shortened or curtailed to prevent excess deterioration on body condition. It is anticipated that the methods and ruminant feed composition disclosed herein will increase feed efficiency by the ruminant animal and therefor reduce the impact of milk production on body condition. As a result, it would be possible to milk cows for a longer duration.

It is also anticipated that the present invention could also be used to reduce or ameliorate the deterioration of body condition due to lactation. It is anticipated that the methods and ruminant feed compositions disclosed herein will increase feed efficiency by the ruminant animal and therefore result in the ruminant animal having an improved body condition at the end of lactation. For example, the animal has a higher body condition score (BCS) when the animal enters the dry period. As a result, the ruminant animal would require less dry matter intake during the offseason to gain body condition. Alternatively or additionally, the methods and ruminant feed compositions disclosed herein are useful for improving body condition of an animal prior to lactation. For example, the methods and compositions disclosed herein could improve the body composition of the mother and/or the foetus or neonate. For example, the methods and compositions disclosed herein could improve body composition and/or weight of the neonate at birth.

It is also anticipated that the methods of the present invention could be used to reduce or ameliorate the deterioration of body condition due to birthing or laying. It is anticipated that the methods disclosed herein will increase feed efficiency by the monogastric animal and therefore result in the monogastric animal having an improved body condition at the end of birthing or laying. As a result, the monogastric animal would require less feed intake to gain body condition. Alternatively or additionally, the methods and feed compositions disclosed herein are useful for improving body condition of an animal prior to lactation. For example, the methods and compositions disclosed herein could improve the body composition of the mother and/or the foetus or neonate. For example, the methods and compositions disclosed herein could improve body composition and/or weight of the neonate at birth.

It is also anticipated that the present invention could be similarly useful for reducing or ameliorating the deterioration of body condition in other times of stress, such as calving, drought, or insufficient feed intake.

As discussed above, the methods and compositions disclosed herein enhance the physical and/or functional development of the rumen, particularly in early life of young or pre-weaning ruminants. The development of the rumen involves three distinct processes: (i) anatomical development (e.g., growth in rumen mass and growth of rumen papillae), (ii) functional achievement (e.g., fermentation capacity and enzyme activity), and (iii) microbial colonization (bacteria, fungi, methanogens, and protozoa).

The anatomical development of the rumen is a process that occurs following three phases: non-rumination (0-3 weeks), transitional phase (3-8 weeks), and rumination (from 8 weeks on. During the transitional phase, growth and development of the ruminal absorptive surface area (papillae) is essential to enable absorption and utilisation of digestion end products, specifically rumen volatile fatty acids. The presence and absorption of volatile fatty acids stimulates rumen epithelial metabolism and may be key in initiating rumen epithelial development. A continuous exposure to volatile fatty acids maintains rumen papillae growth, size, and function. Different volatile fatty acids stimulate such development differently, with butyrate the most stimulatory, followed by propionate. Thus, it is expected that shifts hydrogen metabolism from methanogenesis to short chain/volatile fatty acid (VFA) production, for example to propionic acid production, would therefore enhance rumen epithelial growth and development.

As used herein, the term “gastrointestinal tract” refers to the part of the digestive system that begins in the stomach and ends in the rectum, including the small intestine. Therefore, the mouth and oesophagus are not considered part of the gastrointestinal tract for the purposes of this application.

In some embodiments, the growth of methane-producing bacteria and/or archaea is inhibited in faeces of the animal. In some embodiments, the growth of methane-producing bacteria and/or archaea is inhibited in the distal intestine of the animal. In some embodiments the growth of methane-producing bacteria and/or archaea is inhibited in the colon of the animal. In some embodiments, the growth of methane-producing bacteria and/or archaea is inhibited in the rectum of the animal. In some embodiments, the growth of methane-producing bacteria and/or archaea is inhibited in the small intestine of the animal. In some embodiments, the growth of methane-producing bacteria and/or archaea is inhibited in the hindgut of the animal. In some embodiments, the growth of methane-producing bacteria and/or archaea is inhibited in the caecum of the animal.

It is also anticipated that the methods of the present invention could be useful for improving gut comfort, or preventing, reducing or ameliorating the symptoms caused by gases produced by methanogens in the gastrointestinal tract of the animal, for example excessive flatulence, abdominal distension (bloating) and abdominal pain.

### Ruminants

In some embodiments, the food or feed composition is a ruminant feed composition. In some embodiments, the animal is a ruminant animal.

Ruminants are a group of herbivores having a stomach comprising multiple compartments, that digest their food by a first microbial fermentation in the rumen to form a cud, regurgitating and chewing the cud, and then swallowing the chewed cud for further digestion. This group includes, but is not limited to, the Ruminantia and Tylopoda suborders, and includes several species of domesticated livestock. In one embodiment, the ruminant animal is a bovine, goat, sheep, bison, yak, water buffalo, deer, camel, alpaca, llama, wildebeest, antelope, or nilgai. In a preferred embodiment, the ruminant animal is a bovine or a sheep.

In one embodiment, the ruminant animal is a lactating animal. In an alternative embodiment, the ruminant animal is a pre-weaning animal, such as a calf or a lamb.

The ruminant stomach is divided into the nonglandular forestomach (rumen, reticulum, omasum) and the terminal glandular stomach, the abomasum.

In some embodiments, the ruminant animal is neonatal, newborn, or young. For example, in some embodiments, the ruminant animal is one day, 2 days, 3 days, 4 days, 5 days, 6 days, 1 week, 2 weeks, 3 weeks, one month, or 2 months of age.

In some embodiments, L. rhamnosus strain FNZ129 or a derivative thereof is administered to the ruminant animal prior to weaning. In some embodiments, the L. rhamnosus FNZ129 or derivative thereof is administered to the ruminant animal after weaning. In some embodiments, the L. rhamnosus FNZ129 or derivative thereof is administered to the ruminant animal both prior to weaning and after weaning. For example, in some embodiments, L. rhamnosus strain FNZ129 or a derivative thereof is administered throughout the ruminant animal's life.

For example, the L. rhamnosus FNZ129 or derivative thereof is administered to the ruminant animal on or about day 0 of birth, for example around day 0, day 1 or day 2 of birth. Administration may then occur at least one per day, for example multiple times per day, sufficient to obtain persistency of effect. For example, administration may continue for 2 days, 3 days, 4 days, 5 days, 6 days, 1 week, 2 weeks, 3 weeks, one month, 6 weeks, 2 months, 10 weeks or three months or more from birth. In some embodiments, the administration of L. rhamnosus strain FNZ129 or a derivative thereof continues for four months, five months, six months, seven months, eight months, nine months, ten months, eleven months, 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, or for the life of the animal.

### Monogastric Animals

In some embodiments, the food or feed composition is a feed composition for a monogastric animal. In some embodiments, the animal is a monogastric animal.

Monogastric animals are a group of animals having a simple single-chambered stomach, in comparison with ruminant animals, which have a stomach comprising multiple compartments including a foregut or rumen. The monogastric animals group includes carnivores, omnivores, and herbivores, such as humans, cats, dogs, pigs, horses, donkeys, rabbits, and poultry.

Monogastric animals include several species of domesticated livestock. In one embodiment, the monogastric animal is a human, pig, horse, donkey, rabbit, or poultry. In a preferred embodiment, the monogastric animal is a pig. In one embodiment, the monogastric animal is a chicken, duck, goose or turkey. In one embodiment, the monogastric animal is a companion animal, such as a cat or a dog.

In one embodiment, the monogastric animal is a pre-weaning animal, such as a piglet or a foal. In some embodiments, the L. rhamnosus FNZ129 or derivative thereof is administered to the monogastric animal prior to weaning. In some embodiments, the L. rhamnosus FNZ129 or derivative thereof is administered to the monogastric animal after weaning. In some embodiments, the L. rhamnosus FNZ129 or derivative thereof is administered to the monogastric animal both prior to weaning and after weaning. For example, in some embodiments, L. rhamnosus strain FNZ129 or a derivative thereof is administered throughout the animal's life.

For example, the L. rhamnosus FNZ129 or derivative thereof is administered to the animal on or about day 0 of birth, for example around day 0, day 1 or day 2 of birth. Administration may then occur at least one per day, for example multiple times per day, sufficient to obtain persistency of effect. For example, administration may continue for 2 days, 3 days, 4 days, 5 days, 6 days, 1 week, 2 weeks, 3 weeks, one month, 6 weeks, 2 months, 10 weeks or three months or more from birth. In some embodiments, the administration of L. rhamnosus strain FNZ129 or a derivative thereof continues for four months, five months, six months, seven months, eight months, nine months, ten months, eleven months, 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, or for the life of the animal.

Lacticaseibacillus rhamnosus FNZ129

A culture of Lacticaseibacillus rhamnosus FNZ129 (also known as Lactobacillus rhamnosus FNZ129) was isolated from a human source and deposited at the National Measurement Institute of Australia (NMIA), 1/153 Bertie Street, Port Melbourne, Victoria, Australia 3207 on 2 Aug. 2021, and was given accession number V21/015446. This is a recognised International Depositary Authority under the Budapest Treaty on the International Recognition of the Deposit of Microorganisms for the Purposes of Patent Procedure. The terms Lactobacillus rhamnosus strain FNZ129, Lactobacillus rhamnosus FNZ129, Lacticaseibacillus rhamnosus FNZ129, and L. rhamnosus FNZ129 are used interchangeably herein.

Whole genome sequencing using a combination of short-read (Illumina) and long-read (PacBio) sequencing technologies was used to create hybrid genome assemblies. The final hybrid assembly contained two contigs. Total length was 3094881 bp (3.09 Mb). Species ID was confirmed for the FNZ129 strain as Lacticaseibacillus rhamnosus using the taxonomic sequence classifier programme, Kraken.

All WGS and associated bioinformatics were conducted in accordance with the EFSA Guidance available at: https://efsa.onlinelibrary.wiley.com/doi/pdf/10.2903/j.efsa.2018.5206 in conjunction with the latest EFSA statement (July 2021) available at: https://efsa.onlinelibrary.wiley.com/doi/full/10.2903/j.efsa.2021.6506

### Morphological Properties

The morphological properties of L. rhamnosus FNZ129 are described below.

Short to medium rods with square ends in chains, generally 0.7×1.1×2.0-4.0 μm, when grown in MRS broth. Gram positive, non-mobile, non-spore forming, catalase negative facultative anaerobic rods.

### Further Characterisation

It will be appreciated that there are a wide variety of methods known and available to the skilled artisan that can be used to confirm the identity of L. rhamnosus FNZ129, wherein exemplary methods include DNA fingerprinting, genomic analysis, sequencing, and related genomic and proteomic techniques.

L. rhamnosus Strain FNZ129 and Derivatives Thereof

As described herein, certain embodiments of the present invention utilise live L. rhamnosus strain FNZ129. In other embodiments, a derivative of L. rhamnosus strain FNZ129 is utilised.

As used herein, the term “derivative” and grammatical equivalents thereof when used with reference to bacteria (including use with reference to a specific strain of bacteria such as L. rhamnosus FNZ129) contemplates mutants and homologues of or derived from the bacteria, killed or attenuated bacteria such as but not limited to heat-killed, lysed, fractionated, pressure-killed, irradiated, and UV- or light-treated bacteria, and material derived from the bacteria including but not limited to bacterial cell wall compositions, bacterial cell lysates, lyophilised bacteria, anti-methanogen factors from the bacteria, bacterial metabolites, bacterial cell suspensions, bacterial culture supernatant, and the like, wherein the derivative retains anti-methanogen activity. Transgenic microorganisms engineered to express one or more anti-methanogen factors are also contemplated. Methods to produce such derivatives, such as but not limited to one or more mutants of L. rhamnosus strain FNZ129 or one or more anti-methanogen factors, and particularly derivatives suitable for administration to a ruminant animal (for example, in a composition) are well known in the art.

It will be appreciated that methods suitable for identifying L. rhamnosus strain FNZ129, such as those described above, are similarly suitable for identifying derivatives of L. rhamnosus strain FNZ129, including for example mutants or homologues of L. rhamnosus strain FNZ129, or for example bacterial metabolites from L. rhamnosus strain FNZ129.

The term “anti-methanogen factor” refers to a bacterial molecule responsible for mediating anti-methanogen activity, including but not limited to bacterial DNA motifs, RNA including mRNA and miRNA, proteins, exosomes, bacteriocins, bacteriocin-like molecules, anti-microbial peptides, antibiotics, antimicrobials, small molecules, polysaccharides, or cell wall components such as lipoteichoic acids and peptidoglycan, or a mixture of any two or more thereof. While, as noted above, these molecules have not been clearly identified, and without wishing to be bound by any theory, their presence can be inferred by the presence of anti-methanogen activity.

The term “anti-methanogen activity” refers to the ability of certain microorganisms to inhibit the growth of methanogenic bacteria and/or archaea, and/or to reduce the production of methane by methanogenic bacteria and/or archaea. This ability may be limited to inhibiting the growth of and/or ability to produce methane of certain groups of methanogenic bacteria and/or archaea such as, for example, inhibiting the growth of hydrogenotrophic methanogens, inhibiting the ability of hydrogenotrophic methanogens to produce methane, inhibiting the growth of methylotrophic methanogens, inhibiting the ability of methylotrophic methanogens to produce methane, inhibiting the growth of certain species of methanogens, or inhibiting the ability of certain species of methanogens to produce methane.

Reference to retaining anti-methanogen activity is intended to mean that a derivative of a microorganism, such as a mutant or homologue of a microorganism or an attenuated or killed microorganism, or a cell culture supernatant, still has useful anti-methanogen activity, or that a composition comprising a microorganism or a derivative thereof still has useful anti-methanogen activity. While the bacterial molecules responsible for mediating anti-methanogen activity have not been clearly identified, molecules that have been proposed as possible candidates include bacterial DNA motifs, RNA including mRNA and miRNA, proteins, exosomes, bacteriocins, antibiotics, surface proteins, small organic acids, polysaccharides, and cell wall components such as lipoteichoic acids and peptidoglycan. It has been postulated that these interact with components of the methanogenic bacteria and/or archaea to give a growth-inhibitory effect. Preferably, the retained activity is at least about 35, 40, 45, 50, 55, 60, 65, 70, 75, 80, 85, 90, 95, 99 or 100% of the activity of an untreated (i.e., live or non-attenuated) control, and useful ranges may be selected between any of these values (for example, from about 35 to about 100%, from about 50 to about 100%, from about 60 to about 100%, from about 70 to about 100%, from about 80 to about 100%, and from about 90 to about 100%).

Using conventional solid substrate and liquid fermentation technologies well known in the art, L. rhamnosus strain FNZ129 can be grown in sufficient amounts to allow use as contemplated herein. For example, L. rhamnosus strain FNZ129 can be produced in bulk for formulation using nutrient film or submerged culture growing techniques, for example under conditions as described in WO99/10476. Briefly, growth is carried out under aerobic conditions at any temperature satisfactory for growth of the organism. For example, for L. rhamnosus strain FNZ129, a temperature range of from 30 to 40° C., preferably 37° C., is preferred. The pH of the growth medium is slightly acidic, preferably about 6.0 to 6.5. Incubation time is sufficient for the isolate to reach a stationary growth phase.

Bacterial cells may be harvested by methods well known in the art, for example, by conventional filtering or sedimentary methodologies (e.g. centrifugation) or harvested dry using a cyclone system. Bacterial cells can be used immediately or stored, preferably freeze-dried or chilled at −20° to 6° C., preferably −4° C., for as long as required using standard techniques. Cryoprotectants, cryopreservatives, and/or lyoprotectants may also be used to enhance the stability and/or viability of bacterial cells when dried and/or frozen as is known in the art.

### Supernatants

Further embodiments of the present invention utilise supernatant(s) from a cell culture comprising L. rhamnosus strain FNZ129 or a derivative thereof. These embodiments include processes for preparing a bacterial culture supernatant, said process comprising culturing bacterial cells, and separating the supernatant from the cultured cells, thereby obtaining the supernatant. This process also enables further isolation of bacterial molecules responsible for mediating anti-methanogen activity that are obtainable from the supernatant.

As would be understood by the skilled addressee, a supernatant useful in the present invention encompasses both the supernatant from such cultures, and/or concentrates of such supernatant and/or fractions of such supernatant.

The term “supernatant” in the present context refers to a medium from a bacterial culture from which the bacteria have subsequently been removed, e.g. by centrifugation or filtration.

A supernatant useful in the present invention can readily be obtained by a simple process for preparing a bacterial culture supernatant, said process comprising
a) culturing cells of L. rhamnosus strain FNZ129,
b) optionally releasing of active compounds and/or extracellular components of the cells by various cellular treatments such as, but not limited to, acidic or alkaline modifications, sonication, detergents e.g. Sodium dodecyl sulfate (SDS) and/or Triton X, muralytic enzymes e.g. mutalolysin and/or lysozyme, salt and/or alcohol, and
c) separating the supernatant from the cultured cells,
thereby obtaining said supernatant.

In a preferred embodiment of this process, the supernatant composition is further subjected to a drying step to obtain a dried culture product.

The drying step may conveniently be freeze drying or spray drying, but any drying process which is suitable for drying of anti-methanogen factors such as bacteriocins, also including vacuum drying and air drying, are contemplated.

Although the content of the supernatants produced by L. rhamnosus strain FNZ129 is not yet characterised in detail, it is known that certain bacterial strains may produce bacteriocins that are small heat-stable proteins and therefore, without wishing to be bound by theory, it is expected that even drying methods, including spray drying, which result in moderate heating of the culture eluate product, will result in active compositions, as demonstrated in the Examples described herein.

### Lysate

A fluid containing the contents of lysed cells is called a lysate. A lysate contains active components of the bacterial cells and may be either crude, thus containing all cellular components, or partially and/or completely separated in separate fractions, such as extracellular components, intracellular components, proteins etc.

Methods for producing bacterial cell lysates are well known in the art. Such methods can include, but are not limited to, mechanical lysis, such as mechanical shearing, grinding, milling, or sonication, enzymatic lysis, such as by enzymes that degrade the bacterial cell wall, chemical lysis, such as using detergents, denaturants, pressure alterations, and/or osmotic shock, and combinations of the above.

Further embodiments of the present invention thus utilise a lysate of L. rhamnosus strain FNZ129 or a derivative thereof.

### Cell Suspension

The present invention may also in some embodiments utilise a cell suspension comprising L. rhamnosus strain FNZ129 or a derivative thereof.

In the present context, the term “cell suspension” relates to a number of cells of L. rhamnosus strain FNZ129 or a derivative thereof dispersed or in suspension in a liquid e.g. a liquid nutrient medium, culture medium or saline solution.

The cells may be presented in the form of a cell suspension in a solution that is suitable for dispersion. The cell suspension can e.g. be dispersed via spraying, dipping, or any other application process.

The cells may be viable, but the suspension may also comprise inactivated or killed cells or a lysate hereof. In one embodiment, the suspension of the present invention comprises viable cells. In another embodiment, the suspension of the present invention comprises inactivated, killed or lysed cells.

### Bacteriocins

Bacteriocins are antimicrobial compounds produced by bacteria to inhibit other bacterial strains and species.

Lactic acid bacteria (LAB) are well known to produce bacteriocins and these compounds are of global interest to the food industry because they inhibit the growth of many spoilage and pathogenic bacteria, thus extending shelf life and safety of foods. Bacteriocins are typically considered to be narrow spectrum antibiotics. Moreover, bacteriocins of especially LAB display very low human toxicity and have been consumed in fermented food for millennia.

A further aspect of the invention provides an isolated antimicrobial compound obtainable from L. rhamnosus strain FNZ129 or a derivative thereof. Such antimicrobial compound may for example be obtained from a supernatant or lysate resulting from the process described herein, further comprising an isolation step.

As is illustrated in the Examples disclosed herein, it has been found that L. rhamnosus strain FNZ129 and/or compositions comprising L. rhamnosus strain FNZ129, and/or the culture supernatant of L. rhamnosus strain FNZ129 are useful as an antimicrobial compound, in particular for inhibiting the growth of methane-producing bacteria and/or inhibiting the ability of methanogens to produce methane.

In the present context, the term antimicrobial compound utilises a compound that kills microorganisms, impair their survival or inhibits their growth.

Antimicrobial compounds can be grouped according to the microorganisms they act primarily against. For example, antibacterials are used against bacteria and antifungals are used against fungi. They can also be classified according to their function. Compounds that kill microbes are called microbicidal, while those that merely inhibit their growth are called microbiostatic.

In one embodiment, the present invention relates to an antimicrobial compound, which is microbicidal. In another embodiment, the present invention relates to an antimicrobial compound, which is microbiostatic. In another embodiment, the present invention relates to an antimicrobial compound, which is antibacterial.

### Feed or Carrier Compositions

A feed composition (such as a ruminant feed composition or a monogastric feed composition) useful herein may be formulated as a food, drink, food additive, drink additive, animal feed, animal feed additive, animal feed supplement, dietary supplement, carrier, vitamin or mineral premix, nutritional product, enteral feeding product, soluble, slurry, supplement, pharmaceutical, lick block, drench, tablet, capsule, pellet or intra-ruminal product, e.g., a bolus. Appropriate formulations may be prepared by an art skilled worker with regard to that skill and the teaching of this specification.

The composition can be administered as a top dressing on, or mixed into, a standard feed material such as a daily ration. In addition, the strain can be administered in a partial or total mixed ration (TMR), pelleted feedstuff, mixed in with liquid feed or drink, mixed in a protein premix, or delivered via a vitamin and mineral premix.

In one embodiment, compositions useful herein include any edible feed product which is able to carry bacteria or a bacterial derivative. As used in this application, the term “feed(s)” or “animal feed(s)” refers to material(s) that are consumed by animals and contribute energy and/or nutrients to an animal's diet. Animal feeds typically include a number of different components that may be present in forms such as concentrate(s), premix(es), co-product(s), or pellets. Examples of feeds and feed components include Partial or Total Mixed Ration (TMR), corn, soybean, forage, grain, distiller's grain, sprouted grain, legumes, vitamins, amino acids, minerals, fibre, fodder, grass, hay, straw, silage, kernel, leaves, meal, solubles, slurries, supplements, mash feed, meal, fruit pulp, vegetable pulp, fruit or vegetable pomace, citrus meal, wheat shorts, corn cob meal, and molasses. Other compositions useful as a carrier include milk, milk powder, milk replacement, milk fortifier, colostrum, whey, whey powder, sucrose, maltodextrin, rice hulls and the like.

In certain embodiments, the feed composition is formed through a process of growing L. rhamnosus strain FNZ129 using a milk-based carrier, such as thermalized milk, or a non-milk-based carrier, to create a fermented yoghurt-style composition. Methods to create such fermented yoghurt-style compositions are well known in the art, and may include, for example, using a warm water bath or other heating means to incubate the milk at a suitable temperature until a sufficient cell density is reached, such as over 12 hours. In one embodiment, the temperature is 25-30° C. Optionally, the milk may include other additives to promote bacterial growth, such as yeast extract. In certain embodiments, this method takes place on-site, such as on the farm where the probiotic feed supplementation is to take place. The fermented yoghurt-style composition may be administered by oral application, such as by drenching. In some embodiments, the fermented yoghurt-style composition is administered at a dose of 1-100 ml per day, such as 2-50, 5-30, or 10-20 ml per day.

Other suitable feed formulations for ruminants are described in E. W. Crampton et al., Applied Animal Nutrition, W. H. Freeman and Company, San Francisco, CA., 1969 and D. C. Church, Livestock Feeds and Feeding, 0 & B Books, Corvallis, Oreg., 1977, both of which are incorporated herein by reference.

In one embodiment, compositions useful herein include any non-feed carrier consumed by the animal to which bacteria or a bacterial derivative is added, such as vermiculite, zeolites or crushed limestone and the like.

In one embodiment, compositions useful herein include pet food compositions for companion animals such as cats and dogs. In certain embodiments, the L. rhamnosus FNZ129 is included in the pet food in an amount of about 104 cfu (colony forming units)/g of pet food to about 10¹⁴ cfu/g of pet food. In certain embodiments, the composition further comprises at least one protein source. In certain embodiments, the composition further comprises at least one source of fat. In certain embodiments, the composition further comprises at least one carbohydrate source. In certain embodiments, the pet food is a dog food. In certain embodiments, the pet food is a cat food.

The terms “pet food”, or “pet food composition” as used herein means nutritional compositions intended for ingestion by a pet. In one embodiment a nutritional composition may refer to a dietary supplement intended for ingestion by a pet. A dietary supplement is intended to refer to a composition that provides nutrients that may otherwise not be consumed in sufficient quantities by the pet. In one embodiment a nutritional composition may refer to a pet treat intended for ingestion by a pet. The term “pet treat” as used herein refers to a food for consumption by a pet that is intended as an occasional reward or indulgence and not as the sole source of a pet's nutrition.

In one embodiment, compositions useful herein include food compositions for omnivores such as chickens, pigs, humans, and dogs. Such food compositions are well known in the art.

In certain embodiments, the composition of the invention comprises live L. rhamnosus strain FNZ129. Methods to produce such compositions are well known in the art.

In some embodiments, the composition of the invention comprises one or more derivatives of L. rhamnosus strain FNZ129. Again, methods to produce such compositions are well known in the art and may utilise standard microbiological and pharmaceutical practices. In some embodiments, the composition comprises a dried culture product, such as a supernatant or cell lysate as described herein.

It will be appreciated that a broad range of additives or carriers may be included in such compositions, for example to improve or preserve bacterial viability or to increase anti-methanogen activity of L. rhamnosus strain FNZ129 or a derivative thereof. For example, additives such as surfactants, wetters, humectants, stickers, dispersal agents, stabilisers, penetrants, and so-called stressing additives to improve bacterial cell vigour, growth, replication and survivability (such as potassium chloride, glycerol, sodium chloride and glucose), as well as cryoprotectants such as maltodextrin, may be included. Additives may also include compositions which assist in maintaining microorganism viability in long term storage, for example unrefined corn oil, or “invert” emulsions containing a mixture of oils and waxes on the outside and water, sodium alginate and bacteria on the inside.

In some embodiments, the L. rhamnosus FNZ129 or derivative thereof are encapsulated. Methods to produce such encapsulated bacteria are well known in the art. In some embodiments, the L. rhamnosus FNZ129 or derivative thereof are encapsulated in liposomes, microbubbles, microparticles or microcapsules and the like. Such encapsulants can include natural, semisynthetic, or synthetic polymers, waxes, lipids, fats, fatty alcohols, fatty acids, and/or plasticisers, for example alginates, gums, K-Carrageenan, chitosan, starch, sugars, gelatine, and so on.

In certain embodiments, the L. rhamnosus strain FNZ129 is in a reproductively viable form and amount.

The composition may comprise a carbohydrate source, such as a disaccharide including, for example, sucrose, fructose, glucose, or dextrose. Preferably the carbohydrate source is one able to be aerobically or anaerobically utilised by L. rhamnosus strain FNZ129.

In such embodiments, the composition preferably is capable of supporting reproductive viability of the L. rhamnosus strain FNZ129 for a period greater than about two weeks, preferably greater than about one month, about two months, about three months, about four months, about five months, more preferably greater than about six months, most preferably at least about 2 years to about 3 years or more.

In certain embodiments, an oral composition is formulated to allow the administration of an effective amount of L. rhamnosus strain FNZ129 to establish a population in the gastrointestinal tract of the animal when ingested. The established population may be a transient or permanent population.

While various routes and methods of administration are contemplated, oral administration of L. rhamnosus strain FNZ129, such as in a composition suitable for oral administration, is currently preferred. It will of course be appreciated that other routes and methods of administration may be utilised or preferred in certain circumstances.

The term “oral administration” includes oral, buccal, enteral, intra-ruminal, and intra-gastric administration.

In theory one colony forming unit (cfu) should be sufficient to establish a population of L. rhamnosus strain FNZ129 in an animal, but in actual situations a minimum number of units are required to do so. Therefore, for therapeutic mechanisms that are reliant on a viable, living population of probiotic bacteria, the number of units administered to a subject will affect efficacy.

In one embodiment, a composition formulated for administration will be sufficient to provide at least about 6×10⁹ cfu L. rhamnosus strain FNZ129 per day, for example at least about 6×10¹¹ cfu per day. In another embodiment, a composition formulated for administration will be sufficient to provide at least about 10¹⁰ cfu L. rhamnosus FNZ129 per day. In another embodiment, a composition formulated for administration will be sufficient to provide at least about 10¹² cfu L. rhamnosus strain FNZ129 per day.

Methods to determine the presence of a population of gut and/or rumen flora, such as L. rhamnosus strain FNZ129, in the gastrointestinal tract of a subject are well known in the art, and examples of such methods are presented herein. In certain embodiments, presence of a population of L. rhamnosus strain FNZ129 can be determined directly, for example by analysing one or more samples obtained from an animal and determining the presence or amount of L. rhamnosus strain FNZ129 in said sample. In other embodiments, presence of a population of L. rhamnosus strain FNZ129 can be determined indirectly, for example by observing a reduction in methane emissions or methane production, a reduction in hydrogen production, or a decrease in the number of other gut and/or rumen flora in a sample obtained from an animal. Combinations of such methods are also envisaged.

The efficacy of a composition useful according to the invention can be evaluated both in vitro and in vivo. See, for example, the examples below. Briefly, the composition can be tested for its ability to inhibit the growth of methanogenic bacteria and/or archaea, or its ability to reduce the production of methane by methanogenic bacteria and/or archaea. For in vivo studies, the composition can be fed to or injected into a ruminant or monogastric animal and its effects on methanogenic bacteria and/or archaea, and its effect on methane emissions are then assessed. Based on the results, an appropriate dosage range and administration route can be determined.

Methods of calculating appropriate dose may depend on the nature of the active agent in the composition. For example, when the composition comprises live bacteria, the dose may be calculated with reference to the number of live bacteria present. For example, as described herein the examples the dose may be established by reference to the number of colony forming units (cfu) to be administered per day, or by reference to the number of cfu per kilogram dry feed weight.

By way of general example, the administration of from about 1×10⁶ cfu to about 1×10¹² cfu of L. rhamnosus strain FNZ129 per kg dry feed weight per day, preferably about 1×10⁶ cfu to about 1×10¹¹cfu/kg/day, about 1×10⁶ cfu to about 1×10¹⁰ cfu/kg/day, about 1×10⁶ cfu to about 1×10⁹ cfu/kg/day, about 1×10⁶ cfu to about 1×10⁸ cfu/kg/day, about 1×10⁶ cfu to about 5×10⁷ cfu/kg/day, or about 1×10⁶ cfu to about 1×10⁷ cfu/kg/day, is contemplated. Preferably, the administration of from about 5×10⁶ cfu to about 5×10⁸ cfu per kg dry feed weight of L. rhamnosus strain FNZ129 per day, preferably about 5×10⁶ cfu to about 4×10⁸ cfu/kg/day, about 5×10⁶ cfu to about 3×10⁸ cfu/kg/day, about 5×10⁶ cfu to about 2×10⁸ cfu/kg/day, about 5×10⁶ cfu to about 1×10⁸ cfu/kg/day, about 5×10⁶ cfu to about 9×10⁷ cfu/kg/day, about 5×10⁶ cfu to about 8×10⁷ cfu/kg/day, about 5×10⁶ cfu to about 7×10⁷ cfu/kg/day, about 5×10⁶ cfu to about 6×10⁷ cfu/kg/day, about 5×10⁶ cfu to about 5×10⁷ cfu/kg/day, about 5×10⁶ cfu to about 4×10⁷ cfu/kg/day, about 5×10⁶ cfu to about 3×10⁷ cfu/kg/day, about 5×10⁶ cfu to about 2×10⁷ cfu/kg/day, or about 5×10⁶ cfu to about 1×10⁷ cfu/kg/day, is contemplated.

In certain embodiments, periodic dose need not vary with body weight, dry feed weight or other characteristics of the subject. In such examples, the administration of from about 1×10⁶ cfu to about 1×10¹³ cfu of L. rhamnosus strain FNZ129 per day, preferably about 1×10⁶ cfu to about 1×10¹² cfu/day, about 1×10⁶ cfu to about 1×10¹¹ cfu/day, about 1×10⁶ cfu to about 1×10¹⁰ cfu/day, about 1×10⁶ cfu to about 1×10⁹ cfu/day, about 1×10⁶ cfu to about 1×10⁸ cfu/day, about 1×10⁶ cfu to about 5×10⁷ cfu/day, or about 1×10⁶ cfu to about 1×10⁷ cfu/day, is contemplated.

In certain embodiments, the administration of from about 5×10⁷ cfu to about 5×10¹⁰ cfu per kg body weight of L. rhamnosus strain FNZ129 per day, preferably about 5×10⁷ cfu to about 4×10¹⁰ cfu/day, about 5×10⁷ cfu to about 3×10¹⁰ cfu/day, about 5×10⁷ cfu to about 2×10¹⁰ cfu/day, about 5×10⁷ cfu to about 1×10¹⁰ cfu/day, about 5×10⁷ cfu to about 9×10⁹ cfu/day, about 5×10⁷ cfu to about 8×10⁹ cfu/day, about 5×10⁷ cfu to about 7×10⁹ cfu/day, about 5×10⁷ cfu to about 6×10⁹ cfu/day, about 5×10⁷ cfu to about 5×10⁹ cfu/day, about 5×10⁷ cfu to about 4×10⁹ cfu/day, about 5×10⁷ cfu to about 3×10⁹ cfu/day, about 5×10⁷ cfu to about 2×10⁹ cfu/day, or about 5×10⁷ cfu to about 1×10⁹ cfu/day, is contemplated. Preferably, a dose of between 1×10⁸ and 1×10⁹ cfu/kg body weight per day is administered.

It will be appreciated that, in certain embodiments, the dose need not be administered daily. For example, the composition may be formulated to be administered every two days, twice weekly, weekly, fortnightly, or monthly. Alternatively, in certain embodiments, the composition may be formulated to be administered 2, 3, 4, 5, 6, 7, 8, 9, 10, 11, 12, 13, 14, 15, 16, 17, 18, 19, 20, 21, 22, 23, or 24 times per day, with every feed, or with every mouthful.

In one embodiment, L. rhamnosus FNZ129 may be dosed at between about 1×10³ to about 1×10⁹ cfu/g of pet food and/or pet food admixture.

Suitably, L. rhamnosus FNZ129 may be dosed at between about 1×10⁴ to about 1×10⁸ cfu/g of pet food and/or pet food admixture.

Suitably, L. rhamnosus FNZ129 may be dosed at between about 7.5×10⁴ to about 1×10⁷ cfu/g of pet food and/or pet food admixture.

Preferably, the L. rhamnosus FNZ129 may be dosed at about 1×10⁶ cfu/g of pet food and/or pet food admixture.

In embodiments where the pet food is a pet treat the number of cfu/g dosed may be between about 2 times to about 20 times, suitably between about 4 times to about 15 times the number of cfu/g dosed in a pet food and/or pet food admixture. Preferably the number of cfu/g dosed may be about 10 times the number of cfu/g dosed in a pet food and/or pet food admixture.

It will be appreciated that the composition is preferably formulated so as to allow the administration of an efficacious dose of L. rhamnosus strain FNZ129 and/or one or more derivatives thereof. The dose of the composition administered, the period of administration, and the general administration regime may differ between animals depending on such variables as mode of administration chosen, and the age, sex, body weight, and species of an animal. Furthermore, as described above the appropriate dose may depend on the nature of the active agent in the composition and the manner of formulation.

In some embodiments, the dose of the composition does not vary over time. In other embodiments, the dose of the composition may vary over time. For example, in some embodiments, an initial dosing regimen may be followed by a maintenance dosing regimen.

It will be appreciated that a higher dose may be required to establish a population of L. rhamnosus FNZ129 in the animal, and a lower dose may be sufficient to maintain said population. Accordingly, in some embodiments, the initial dosing regimen comprises administering a higher dose and/or a more frequent dose than the maintenance dosing regimen. Preferably, the initial dosing regimen is efficacious to establish a population of L. rhamnosus FNZ129 in the animal, and preferably the maintenance dosing regimen is efficacious to maintain a population of L. rhamnosus FNZ129 in the animal. In some embodiments, the maintenance dosing regimen comprises administering a dose every day, every second day, twice weekly, weekly, fortnightly, or monthly.

In some embodiments, the effect of the methods described herein persist after the administration of the L. rhamnosus FNZ129. Without wishing to be bound by theory, it is anticipated that administration of L. rhamnosus FNZ129 as described herein may result in a long-lasting or even permanent changes in the forestomach or rumen of the ruminant animal, or the gastrointestinal tract of the monogastric animal. In some embodiments, the effect persists for 2 days after the last administration of L. rhamnosus FNZ129, such as 3 days, 5 days, 1 week, 2 weeks, 1 month, 2 months, 3 months, 4 months, 5 months, 6 months, 7 months 8 months, 9 months, 10 months, 11 months, 1 year, 2 years, 3 years, 4 years, 5 years, 6 years, or 7 years after the last administration of L. rhamnosus FNZ129. In a preferred embodiment, the effect persists for the life of the animal.

In examples where the composition comprises one or more derivatives of L. rhamnosus strain FNZ129, the dose may be calculated by reference to the amount or concentration of the derivative to be administered per day. For example, when the bacteria are inactivated, the quantities described previously are calculated before inactivation. For a composition comprising L. rhamnosus strain FNZ129 culture supernatant, the dose may be calculated by reference to the concentration of culture supernatant present in the composition. The concentration of culture supernatant present in the composition may be calculated, for example, on the basis of the cfu of the culture. For example, a dosage of culture supernatant equivalent to 1×10¹ cfu/day can be calculated from the total yield of the culture and the total volume of the culture supernatant.

It will be appreciated that preferred compositions are formulated to provide an efficacious dose in a convenient form and amount. In certain embodiments, such as but not limited to those where periodic dose need not vary with body weight or other characteristics of the animal, the composition may be formulated for unit dosage. It should be appreciated that administration may include a single daily dose or administration of a number of discrete divided doses as may be appropriate. For example, an efficacious dose of L. rhamnosus strain FNZ129 may be formulated into a feed for oral administration.

However, by way of general example, the inventors contemplate administration of from about 1 mg to about 1000 mg of a composition useful herein per day, preferably about 50 to about 500 mg per day, alternatively about 150 to about 410 mg/day or about 110 to about 310 mg/day. In one embodiment, the inventors contemplate administration of from about 0.05 mg to about 250 mg per kg body weight of a composition useful herein. For example, administration to a human may comprise a single dose of 6×10⁹ CFU per day to adults or children as a 500 mg capsule.

In one embodiment a composition useful herein comprises, consists essentially of, or consists of at least about 0.1, 0.2, 0.5, 1, 5, 10, 15, 20, 25, 30, 35, 40, 45, 50, 55, 60, 65, 70, 75, 80, 85, 90, 95, 99, 99.5, 99.8 or 99.9% by weight of L. rhamnosus strain FNZ129 and/or a derivative thereof and useful ranges may be selected between any of these foregoing values (for example, from about 0.1 to about 50%, from about 0.2 to about 50%, from about 0.5 to about 50%, from about 1 to about 50%, from about 5 to about 50%, from about 10 to about 50%, from about 15 to about 50%, from about 20 to about 50%, from about 25 to about 50%, from about 30 to about 50%, from about 35 to about 50%, from about 40 to about 50%, from about 45 to about 50%, from about 0.1 to about 60%, from about 0.2 to about 60%, from about 0.5 to about 60%, from about 1 to about 60%, from about 5 to about 60%, from about 10 to about 60%, from about 15 to about 60%, from about 20 to about 60%., from about 25 to about 60%, from about 30 to about 60%, from about 35 to about 60%, from about 40 to about 60%, from about 45 to about 60%, from about 0.1 to about 70%, from about 0.2 to about 70%, from about 0.5 to about 70%, from about 1 to about 70%, from about 5 to about 70%, from about 10 to about 70%, from about 15 to about 70%, from about 20 to about 70%, from about 25 to about 70%, from about 30 to about 70%, from about 35 to about 70%, from about 40 to about 70%, from about 45 to about 70%, from about 0.1 to about 80%, from about 0.2 to about 80%, from about 0.5 to about 80%, from about 1 to about 80%, from about 5 to about 80%, from about 10 to about 80%, from about 15 to about 80%, from about 20 to about 80%., from about 25 to about 80%, from about 30 to about 80%, from about 35 to about 80%, from about 40 to about 80%, from about 45 to about 80%, from about 0.1 to about 90%, from about 0.2 to about 90%, from about 0.5 to about 90%, from about 1 to about 90%, from about 5 to about 90%, from about 10 to about 90%, from about 15 to about 90%, from about 20 to about 90%, from about 25 to about 90%, from about 30 to about 90%, from about 35 to about 90%, from about 40 to about 90%, from about 45 to about 90%, from about 0.1 to about 99%, from about 0.2 to about 99%, from about 0.5 to about 99%, from about 1 to about 99%, from about 5 to about 99%, from about 10 to about 99%, from about 15 to about 99%, from about 20 to about 99%, from about 25 to about 99%, from about 30 to about 99%, from about 35 to about 99%, from about 40 to about 99%, and from about 45 to about 99%).

In one embodiment a composition useful herein comprises, consists essentially of, or consists of at least about 0.001, 0.01, 0.05, 0.1, 0.15, 0.2, 0.3, 0.4, 0.5, 1, 2, 3, 4, 5, 6, 7, 8, 9, 10, 11, 12, 13, 14, 15, 16, 17, 18 or 19 grams of L. rhamnosus strain FNZ129 and/or a derivative thereof and useful ranges may be selected between any of these foregoing values (for example, from about 0.01 to about 1 grams, about 0.01 to about 10 grams, about 0.01 to about 19 grams, from about 0.1 to about 1 grams, about 0.1 to about 10 grams, about 0.1 to about 19 grams, from about 1 to about 5 grams, about 1 to about 10 grams, about 1 to about 19 grams, about 5 to about 10 grams, and about 5 to about 19 grams).

In certain embodiments, a composition useful herein comprises, consists essentially of, or consists of at least about 10⁴, 10⁵, 10⁶, 10⁷, 10⁸, 10⁹, 10¹⁰, 10¹¹, 10¹², or 10¹³ colony forming units (cfu) of L. rhamnosus strain FNZ129 per kg dry weight of the composition, and useful ranges may be selected between any of these foregoing values (for example, from about 10⁵ to about 10¹³ cfu, from about 10⁶ to about 10¹² cfu, from about 10⁷ to about 10¹² cfu, from about 10⁸ to about 10¹¹ cfu, from about 10⁸ to about 10¹⁰ cfu, and from about 10⁸ to about 10⁹ cfu).

It will be apparent that the concentration of L. rhamnosus strain FNZ129 and/or one or more derivatives thereof in a composition formulated for administration may be less than that in a composition formulated for, for example, distribution or storage, and that the concentration of a composition formulated for storage and subsequent formulation into a composition suitable for administration must be adequate to allow said composition for administration to also be sufficiently concentrated so as to be able to be administered at an efficacious dose.

The compositions useful herein may be used alone or in combination with one or more other therapeutic agents. The therapeutic agent may be a food, drink, food additive, drink additive, food component, drink component, dietary supplement, vitamin or mineral premix, oil, oil blend, oil rich feed supplement, nutritional product, medical food, nutraceutical, medicament or pharmaceutical. The therapeutic agent may be a probiotic agent or a probiotic factor, and is preferably effective to inhibit the growth of methanogenic bacteria and/or archaea, or to reduce methane emissions by methanogenic bacteria and/or archaea. In some embodiments, the oil, oil blend, or oil rich feed supplement is palm kernel expeller (PKE) and/or PROLIQ.

When used in combination with another therapeutic agent, the administration of a composition useful herein and the other therapeutic agent may be simultaneous or sequential. Simultaneous administration includes the administration of a single dosage form that comprises all components or the administration of separate dosage forms at substantially the same time. Sequential administration includes administration according to different schedules, preferably so that there is an overlap in the periods during which the composition useful herein and other therapeutic agent are provided. Examples of other therapeutic agents include at least one microorganism of a different species or strain, a vaccine that inhibits methanogens or methanogenesis, and/or a natural or chemically-synthesised inhibitor of methanogenesis and/or methanogen inhibitor, such as bromoform.

Suitable agents with which the compositions useful herein can be separately, simultaneously or sequentially administered include one or more prebiotic agents, one or more probiotic agents, one or more postbiotic agents, one or more phospholipids, one or more gangliosides, other suitable agents known in the art, and combinations thereof.

Typically, the term prebiotic refers to a material that stimulates the growth and/or activity of bacteria in the animals' digestive system that have biologic activity. Prebiotics may be selectively fermented ingredients that allow specific changes, both in the composition and/or activity of the gastrointestinal microflora, which confer health benefits upon the host. Probiotics generally refer to microorganisms that contribute to intestinal microbial balance which in turn play a role in maintaining health, or providing other biologic activity. Many species of lactic acid bacteria (LAB) such as, Lacticaseibacillus and Bifidobacterium are generally considered as probiotics, but some species of Bacillus, and some yeasts have also been found as suitable candidates. Postbiotics refer to non-viable bacterial products or metabolic byproducts from microorganisms such as probiotics, that have biologic activity in the host.

Useful prebiotics include galactooligosaccharides (GOS), short chain GOS, long chain GOS, fructooligosaccharides (FOS), short chain FOS, long chain FOS, inulin, galactans, fructans, lactulose, and any mixture of any two or more thereof. Some prebiotics are reviewed by Boehm G and Moro G (Structural and Functional Aspects of Prebiotics Used in Infant Nutrition, J. Nutr. (2008) 138(9):1818S-1828S), incorporated herein by reference. Other useful agents may include dietary fibre such as a fully or partially insoluble or indigestible dietary fibre.

Accordingly, in one embodiment L. rhamnosus strain FNZ129 and/or a derivative thereof may be administered separately, simultaneously or sequentially with one or more agents selected from one or more probiotics, one or more prebiotics, one or more sources of dietary fibre, one or more galactooligosaccharides, one or more short chain galactooligosaccharides, one or more long chain galactooligosaccharides, one or more fructooligosaccharides, one or more short chain galactooligosaccharides, one or more long chain galactooligosaccharides, inulin, one or more galactans, one or more fructans, lactulose, or any mixture of any two or more thereof.

In certain embodiments, the composition comprises L. rhamnosus strain FNZ129 and/or a derivative thereof and one or more prebiotics, one or more probiotics, one or more postbiotics, one or more sources of dietary fibre. In certain embodiments, the prebiotic comprises one or more fructooligosaccharides, one or more galactooligosaccharides, inulin, one or more galactans, one or more fructans, lactulose, or any mixture of any two or more thereof.

Without wishing to be bound by theory, it is believed that co-culture and/or co-administration of two or more strains of lactic acid bacteria, such as three strains of lactic acid bacteria, can reduce the incidence of culture failure due to infection by bacteriophages. Accordingly, in certain embodiments, the composition comprises L. rhamnosus FNZ129 and one or more other strain of lactic acid bacteria, preferably two or more other strains of lactic acid bacteria. In other embodiments, the composition comprising L. rhamnosus FNZ129 is administered simultaneously or sequentially with one or more other compositions comprising one or more other strains of lactic acid bacteria, preferably two or more other strains of lactic acid bacteria.

It will be appreciated that different compositions of the invention may be formulated with a view to administration to a particular subject group, for example a particular ruminant subject group or a particular monogastric subject group. For example, the formulation of a composition suitable to be administered to cattle may differ to that suitable to be administered to a different ruminant, such as sheep; and the formulation of a composition suitable to be administered to pigs be differ to that suitable to be administered to a different monogastric animal, such as horses. It should also be appreciated that compositions of the invention may be formulated differently to be suitable to be administered to animals of different ages. For example, the formulation of a composition suitable to be administered to calves, lambs, piglets, or foals may differ to that suitable to be administered to adult cows, sheep, pigs, or horses. In certain embodiments, a first composition may be formulated for administration to young animals, such as pre-weaning animals, in an initial dosing regimen, and a second composition may be formulated for administration to the same animals in a maintenance dosing regimen. In some embodiments, the first composition is formulated for pre-weaning animals and the second composition is formulated for post-weaning animals.

Preparation of L. rhamnosus Strain FNZ129

Direct-fed microbials (DFMs) and their use in methods to modulate ruminal function and improve ruminant performance is known in the art, as are methods for their production.

Briefly, L. rhamnosus strain FNZ129 can be cultured using conventional liquid or solid fermentation techniques. In at least one embodiment, the strain is grown in a liquid nutrient broth, to a level at which the highest number of cells are formed. The strain is produced by fermenting the bacterial strain, which can be started by scaling-up a seed culture. This involves repeatedly and aseptically transferring the culture to a larger and larger volume to serve as the inoculum for the fermentation, which can be carried out in large stainless steel fermenters in medium containing proteins, carbohydrates, and minerals necessary for optimal growth. Non-limiting exemplary media are MRS or TSB. However, other media can also be used. After the inoculum is added to the fermentation vessel, the temperature and agitation are controlled to allow maximum growth. Once the culture reaches a maximum population density, the culture is harvested by separating the cells from the fermentation medium. This is commonly done by centrifugation.

In one embodiment, to prepare the L. rhamnosus strain FNZ129, the strain is fermented to a density of from about 1×10⁸ CFU/ml to about 1×10⁹ CFU/ml. The bacteria are harvested by centrifugation, and the supernatant is removed. The pelleted bacteria can then be used to produce a DFM. In at least some embodiments, the pelleted bacteria are freeze-dried and then used to form a DFM. However, it is not necessary to freeze-dry the strain before using them. The strain can also be used with or without preservatives, and in concentrated, unconcentrated, or diluted form.

The count of the culture can then be determined. CFU or colony forming unit is the viable cell count of a sample resulting from standard microbiological plating methods. The term is derived from the fact that a single cell when plated on appropriate medium will grow and become a viable colony in the agar medium.

Since multiple cells may give rise to one visible colony, the term colony forming unit is a more useful unit measurement than cell number.

### Particular Embodiments

Particular embodiments of the invention will now be described by way of numbered paragraphs:

A1. Isolated Lacticaseibacillus rhamnosus (Lactobacillus rhamnosus) strain FNZ129, NMIA accession number V21/015446 dated 2 Aug. 2021, or a derivative thereof.

A2. A food or feed composition comprising Lacticaseibacillus rhamnosus (Lactobacillus rhamnosus) strain FNZ129, NMIA accession number V21/015446 dated 2 Aug. 2021, or a derivative thereof.

A3. The food or feed composition of A2, wherein the composition is a ruminant feed composition.

A4. A ruminant feed composition for inhibiting the growth of methane-producing bacteria and/or archaea in the forestomach of ruminant animals, reducing the ability of the rumen microbiome to produce methane, reducing methane emissions by a ruminant animal, increasing feed efficiency in a ruminant animal, enhancing the growth and/or productivity in a ruminant animal, increasing the yield of milk and/or milk components produced from a ruminant animal, or improving the body weight and/or body composition of a ruminant animal, the feed composition comprising Lacticaseibacillus rhamnosus (Lactobacillus rhamnosus) strain FNZ129, NMIA accession number V21/015446 dated 2 Aug. 2021, or a derivative thereof.

A5. The ruminant feed composition of A4, wherein the feed composition a fermented yoghurt-style composition, and wherein the fermented yoghurt-style composition is formed through a process of growing L. rhamnosus FNZ129 using a milk-based carrier or non-milk-based carrier.

A6. The ruminant feed composition of A4, which is or comprises Partial or Total Mixed Ration (TMR), corn, soybean, forage, grain, distiller's grain, sprouted grain, legumes, fibre, fodder, grass, hay, straw, silage, kernel, leaves, meal, mash feed, lick block, or molasses.

A7. The ruminant feed composition of any one of A4 to A6, further comprising at least one microorganism of a different species or strain, a vaccine that inhibits methanogens or methanogenesis, and/or a natural or chemically-synthesised inhibitor of methanogenesis and/or methanogen inhibitor such as bromoform.

A8. The ruminant feed composition of any one of A4 to A7, further comprising one or more agents selected from one or more prebiotics, one or more probiotics, one or more postbiotics, one or more sources of dietary fibre, one or more galactooligosaccharides, one or more short chain galactooligosaccharides, one or more long chain galactooligosaccharides, one or more fructooligosaccharides, inulin, one or more galactans, one or more fructans, lactulose, or any mixture of any two or more thereof.

A9. The ruminant feed composition of any one of A4 to A8, wherein the derivative of the L. rhamnosus FNZ129 is a cell lysate of the strain, a cell suspension of the strain, a metabolite of the strain, a culture supernatant of the strain, or killed L. rhamnosus FNZ129.

A10. A method for inhibiting the growth of methane-producing bacteria and/or archaea in the forestomach of a ruminant animal, said method comprising the step of administering to said animal a food or feed composition as defined in A2 or A3, or a ruminant feed composition as defined in any one of A4 to A9.

A11. A method for reducing the ability of the rumen microbiome of a ruminant animal to produce methane, said method comprising the step of administering to said animal a food or feed composition as defined in A2 or A3, or a ruminant feed composition as defined in any one of A4 to A9.

A12. A method for reducing methane emissions by a ruminant animal, said method comprising the step of administering to said animal a food or feed composition as defined in A2 or A3, or a ruminant feed composition as defined in any one of A4 to A9.

A13. A method for increasing feed efficiency in a ruminant animal, said method comprising the step of administering to said animal a food or feed composition as defined in A2 or A3, or a ruminant feed composition as defined in any one of A4 to A9.

A14. A method for enhancing the growth and/or productivity in a ruminant animal, said method comprising the step of administering to said animal a food or feed composition as defined in A2 or A3, or a ruminant feed composition as defined in any one of A4 to A9.

A15. A method for increasing the yield of milk and/or milk components produced from a ruminant animal, said method comprising the step of administering to said animal a food or feed composition as defined in A2 or A3, or a ruminant feed composition as defined in any one of A4 to A9.

A16. A method for improving the body weight and/or body composition of a ruminant animal, said method comprising the step of administering to said animal a food or feed composition as defined in A2 or A3, or a ruminant feed composition as defined in any one of A4 to A9.

A17. A method for inhibiting the growth of methane-producing bacteria and/or archaea in the forestomach of ruminant animals, wherein the method comprises administering to a ruminant animal an effective amount of Lacticaseibacillus rhamnosus (Lactobacillus rhamnosus) strain FNZ129, NMIA accession number V21/015446 dated 2 Aug. 2021, or a derivative thereof.

A18. A method for reducing methane emissions by a ruminant animal, wherein the method comprises administering to the animal an effective amount of Lacticaseibacillus rhamnosus (Lactobacillus rhamnosus) strain FNZ129, NMIA accession number V21/015446 dated 2 Aug. 2021, or a derivative thereof.

A19. A method for increasing feed efficiency in a ruminant animal, wherein the method comprises administering to the animal an effective amount of Lacticaseibacillus rhamnosus (Lactobacillus rhamnosus) strain FNZ129, NMIA accession number V21/015446 dated 2 Aug. 2021, or a derivative thereof.

A20. A method for enhancing the growth and/or productivity in a ruminant animal, said method comprising the step of administering to said animal an effective amount of Lacticaseibacillus rhamnosus (Lactobacillus rhamnosus) strain FNZ129, NMIA accession number V21/015446 dated 2 Aug. 2021, or a derivative thereof.

A21. A method for increasing the yield of milk and/or milk components produced from a ruminant animal, said method comprising the step of administering to said animal an effective amount of Lacticaseibacillus rhamnosus (Lactobacillus rhamnosus) strain FNZ129, NMIA accession number V21/015446 dated 2 Aug. 2021, or a derivative thereof.

A22. A method for improving the body weight and/or body composition of a ruminant animal, said method comprising the step of administering to said animal an effective amount of Lacticaseibacillus rhamnosus (Lactobacillus rhamnosus) strain FNZ129, NMIA accession number V21/015446 dated 2 Aug. 2021, or a derivative thereof.

A23. A method for reducing the ability of the rumen microbiome to produce methane, wherein the method comprises administering to the animal an effective amount of Lacticaseibacillus rhamnosus (Lactobacillus rhamnosus) strain FNZ129, NMIA accession number V21/015446 dated 2 Aug. 2021, or a derivative thereof.

A24. The method of any one of A17 to A23, wherein the method inhibits the growth of methylotrophic methanogens in the forestomach of the animal, preferably a methanogen from the genus Methanosphaera.

A25. The method of any one of A17 to A24, wherein the L. rhamnosus FNZ129 or derivative thereof is administered in a composition that is a food, drink, food additive, drink additive, animal feed, animal feed additive, animal feed supplement, dietary supplement, carrier, vitamin or mineral premix, nutritional product, enteral feeding product, soluble, supplement, pharmaceutical, lick block, drench, tablet, capsule, pellet or intra-ruminal product, e.g., a bolus, or wherein the L. rhamnosus FNZ129 is encapsulated, for example in liposomes, microbubbles, microparticles or microcapsules.

A26. The method of A25, wherein the L. rhamnosus FNZ129 or derivative thereof is administered in drinking water, milk, milk powder, milk replacement, milk fortifier, whey, whey powder, Partial or Total Mixed Ration (TMR), corn, soybean, forage, grain, distiller's grain, sprouted grain, legumes, vitamins, amino acids, minerals, fibre, fodder, grass, hay, straw, silage, kernel, leaves, meal, solubles, supplements, mash feed, meal, fruit pulp, vegetable pulp, fruit or vegetable pomace, citrus meal, wheat shorts, corn cob meal, molasses, sucrose, maltodextrin, rice hulls, vermiculite, zeolites or crushed limestone.

A27. The method of any one of A17 to A26, wherein the method comprises administering to the animal L. rhamnosus FNZ129 in an amount of from 10⁴ to 10¹³ colony forming units per kilogram of dry weight carrier feed, from 10⁴ to 10¹⁰ colony forming units per kilogram of body weight of the animal per day, or from 10⁴ to 10¹³ colony forming units per day.

A28. The method of A27, wherein the method comprises administering to the animal L. rhamnosus FNZ129 in an amount from 10⁸ to 10¹² colony forming units per kilogram of dry weight carrier feed, from 10⁵ to 10⁸ colony forming units per kilogram of body weight of the animal per day, or from 10⁶ to 10¹³ colony forming units per day.

A29. The method of any one of A17 to A28, wherein the derivative of L. rhamnosus FNZ129 is a cell lysate of the strain, a cell suspension of the strain, a metabolite of the strain, a culture supernatant of the strain, or killed L. rhamnosus FNZ129.

A30. The method of any one of A17 to A29, the method comprising further administering at least one microorganism of a different species or strain, a vaccine that inhibits methanogens or methanogenesis, and/or a natural or chemically-synthesised inhibitor of methanogenesis and/or methanogen inhibitor such as bromoform.

A31. The method of any one of A17 to A30, wherein the L. rhamnosus FNZ129 or derivative thereof is administered separately, simultaneously or sequentially with one or more agents selected from one or more prebiotics, one or more probiotics, one or most postbiotics, one or more sources of dietary fibre, one or more galactooligosaccharides, one or more short chain galactooligosaccharides, one or more long chain galactooligosaccharides, one or more fructooligosaccharides, inulin, one or more galactans, one or more fructans, lactulose, or any mixture of any two or more thereof.

A32. The method of any one of A17 to A31, wherein the method enhances the growth or productivity of the ruminant animal.

A33. The method of A32, wherein the method increases the yield of milk and/or milk components produced from the ruminant animal.

A34. The method of A33, wherein the method increases the yield of milk fat, milk protein or milk solids in milk produced from the ruminant animal.

A35. The method of any one of A17 to A34, wherein the method additionally improves the body weight and/or body composition of the ruminant animal.

A36. The method of any one of A17 to A35, wherein said ruminant animal is a bovine, goat, sheep, bison, yak, water buffalo, deer, camel, alpaca, llama, wildebeest, antelope, or nilgai.

A37. The method of any one of A17 to A36, wherein said ruminant animal are cattle or sheep.

A38. The method of any one of A17 to A37, wherein said ruminant animal are cattle.

A39. The method of any one of A17 to A38, wherein said ruminant animal is a lactating animal.

A40. The method of any one of A17 to A38, wherein said ruminant animal is a pre-weaning animal, for example a calf or a lamb.

A41. The method of any one of A17 to A38, wherein said ruminant animal is a post-weaning animal.

A42. The method of any one of A17 to A38, wherein the L. rhamnosus FNZ129 is administered to the ruminant animal both prior to weaning and after weaning.

A43. The method of any one of A17 to A38, wherein the administering is to a pre-weaning animal and wherein the inhibition of the growth of methane-producing bacteria and/or archaea in the forestomach of the ruminant animals, the reduction of methane emissions, for example methane production, by the ruminant animal, and/or the increased feed efficiency in the ruminant animal persists post-weaning.

A44. The method of any one of A17 to A43, wherein the inhibition of the growth of methane-producing bacteria and/or archaea in the forestomach of the ruminant animals, the reduction of methane emissions, for example methane production, by the ruminant animal, and/or the increased feed efficiency in the ruminant animal persists for at least 2 days, 3 days, 5 days, 1 week, 2 weeks, 1 month, 2 months, 3 months, 4 months, 5 months, 6 months, 7 months 8 months, 9 months, 10 months, 11 months, 1 year, 2 years, 3 years, 4 years, 5 years, 6 years, or 7 years after the last administration of L. rhamnosus FNZ129.

A45. The method of A44, wherein the inhibition of the growth of methane-producing bacteria and/or archaea in the forestomach of the ruminant animals, the reduction of methane emissions, for example methane production, by the ruminant animal, and/or the increased feed efficiency in the ruminant animal persists for the life of the ruminant animal.

A46. Use of Lacticaseibacillus rhamnosus (Lactobacillus rhamnosus) strain FNZ129, NMIA accession number V21/015446 dated 2 Aug. 2021, or a derivative thereof, for the manufacture of a composition for inhibiting the growth of methane-producing bacteria and/or archaea in the forestomach of ruminant animals, reducing the ability of the rumen microbiome to produce methane, reducing methane emissions by a ruminant animal, increasing feed efficiency in a ruminant animal, increasing the yield of milk and/or milk components produced from a ruminant animal, or improving the body weight and/or body composition of a ruminant animal.

A47. Use according to A46, wherein the composition comprises a food or feed composition as defined in A2 or A3, or a ruminant feed composition as defined in any one of A4 to A9.

A48. Lacticaseibacillus rhamnosus (Lactobacillus rhamnosus) strain FNZ129, NMIA accession number V21/015446 dated 2 Aug. 2021, or a derivative thereof, for use in inhibiting the growth of methane-producing bacteria and/or archaea in the forestomach of ruminant animals, reducing the ability of the rumen microbiome to produce methane, reducing methane emissions by a ruminant animal, increasing feed efficiency in a ruminant animal, increasing the yield of milk and/or milk components produced from a ruminant animal, or improving the body weight and/or body composition of a ruminant animal.

B1. Isolated Lacticaseibacillus rhamnosus (Lactobacillus rhamnosus) strain FNZ129, NMIA accession number V21/015446 dated 2 Aug. 2021, or a derivative thereof.

B2. A food or feed composition comprising Lacticaseibacillus rhamnosus (Lactobacillus rhamnosus) strain FNZ129, NMIA accession number V21/015446 dated 2 Aug. 2021, or a derivative thereof.

B3. The food or feed composition of B2, wherein the composition is a feed composition for a monogastric animal.

B4. A monogastric feed composition for inhibiting the growth of methane-producing bacteria and/or archaea in the gastrointestinal tract of monogastric animals, reducing the ability of the gastrointestinal microbiome to produce methane, reducing methane emissions by a monogastric animal, increasing feed efficiency in a monogastric animal, enhancing the growth and/or productivity in a monogastric animal, increasing the yield of milk and/or milk components produced from a monogastric animal, or improving the body weight and/or body composition of a monogastric animal, the feed composition comprising Lacticaseibacillus rhamnosus (Lactobacillus rhamnosus) strain FNZ129, NMIA accession number V21/015446 dated 2 Aug. 2021, or a derivative thereof.

B5. The monogastric feed composition of B4, wherein the feed composition a fermented yoghurt-style composition, and wherein the fermented yoghurt-style composition is formed through a process of growing L. rhamnosus FNZ129 using a milk-based carrier or non-milk-based carrier.

B6. The monogastric feed composition of B4, which is or comprises Partial or Total Mixed Ration (TMR), corn, soybean, forage, grain, distiller's grain, sprouted grain, legumes, fibre, fodder, grass, hay, straw, silage, kernel, leaves, meal, mash feed, fruit pulp, vegetable pulp, fruit or vegetable pomace, citrus meal, wheat shorts, corn cob meal, or molasses.

B7. The monogastric feed composition of any one of B4 to B6, further comprising at least one microorganism of a different species or strain, a vaccine that inhibits methanogens or methanogenesis, and/or a natural or chemically-synthesised inhibitor of methanogenesis and/or methanogen inhibitor such as bromoform.

B8. The monogastric feed composition of any one of B4 to B7, further comprising one or more agents selected from one or more prebiotics, one or more probiotics, one or more postbiotics, one or more sources of dietary fibre, one or more galactooligosaccharides, one or more short chain galactooligosaccharides, one or more long chain galactooligosaccharides, one or more fructooligosaccharides, inulin, one or more galactans, one or more fructans, lactulose, or any mixture of any two or more thereof.

B9. The monogastric feed composition of any one of B4 to B8, wherein the derivative of the L. rhamnosus FNZ129 is a cell lysate of the strain, a cell suspension of the strain, a metabolite of the strain, a culture supernatant of the strain, or killed L. rhamnosus FNZ129.

B10. A method for inhibiting the growth of methane-producing bacteria and/or archaea in the gastrointestinal tract of a monogastric animal, said method comprising the step of administering to said animal a food or feed composition as defined in B2 or B3, or a monogastric feed composition as defined in any one of B4 to B9.

B11. A method for reducing the ability of the gastrointestinal microbiome of a monogastric animal to produce methane, said method comprising the step of administering to said animal a food or feed composition as defined in B2 or B3, or a monogastric feed composition as defined in any one of B4 to B9.

B12. A method for reducing methane emissions by a monogastric animal, said method comprising the step of administering to said animal a food or feed composition as defined in B2 or B3, or a monogastric feed composition as defined in any one of B4 to B9.

B13. A method for increasing feed efficiency in a monogastric animal, said method comprising the step of administering to said animal a food or feed composition as defined in B2 or B3, or a monogastric feed composition as defined in any one of B4 to B9.

B14. A method for enhancing the growth and/or productivity in a monogastric animal, said method comprising the step of administering to said animal a food or feed composition as defined in B2 or B3, or a monogastric feed composition as defined in any one of B4 to B9.

B15. A method for increasing the yield of milk and/or milk components produced from a monogastric animal, said method comprising the step of administering to said animal a food or feed composition as defined in B2 or B3, or a monogastric feed composition as defined in any one of B4 to B9.

B16. A method for improving the body weight and/or body composition of a monogastric animal, said method comprising the step of administering to said animal a food or feed composition as defined in B2 or B3, or a monogastric feed composition as defined in any one of B4 to B9.

B17. A method for inhibiting the growth of methane-producing bacteria and/or archaea in the gastrointestinal tract of monogastric animals, wherein the method comprises administering to a monogastric animal an effective amount of Lacticaseibacillus rhamnosus (Lactobacillus rhamnosus) strain FNZ129, NMIA accession number V21/015446 dated 2 Aug. 2021, or a derivative thereof.

B18. A method for reducing methane emissions by a monogastric animal, wherein the method comprises administering to the animal an effective amount of Lacticaseibacillus rhamnosus (Lactobacillus rhamnosus) strain FNZ129, NMIA accession number V21/015446 dated 2 Aug. 2021, or a derivative thereof.

B19. A method for increasing feed efficiency in a monogastric animal, wherein the method comprises administering to the animal an effective amount of Lacticaseibacillus rhamnosus (Lactobacillus rhamnosus) strain FNZ129, NMIA accession number V21/015446 dated 2 Aug. 2021, or a derivative thereof.

B20. A method for reducing the ability of the gastrointestinal microbiome to produce methane, wherein the method comprises administering to the animal an effective amount of Lacticaseibacillus rhamnosus (Lactobacillus rhamnosus) strain FNZ129, NMIA accession number V21/015446 dated 2 Aug. 2021, or a derivative thereof.

B21. A method for enhancing the growth and/or productivity in a monogastric animal, wherein the method comprises administering to a monogastric animal an effective amount of Lacticaseibacillus rhamnosus (Lactobacillus rhamnosus) strain FNZ129, NMIA accession number V21/015446 dated 2 Aug. 2021, or a derivative thereof.

B22. A method for improving the body weight and/or body composition of monogastric animal, wherein the method comprises administering to a monogastric animal an effective amount of Lacticaseibacillus rhamnosus (Lactobacillus rhamnosus) strain FNZ129, NMIA accession number V21/015446 dated 2 Aug. 2021, or a derivative thereof.

B23. The method of any one of B17 to B22, wherein the method inhibits the growth of a methylotrophic methanogen in the caecum of the animal, preferably a methanogen from the genus Methanosphaera.

B24. The method of any one of B17 to B23, wherein the L. rhamnosus FNZ129 or derivative thereof is administered in a composition that is a food, drink, food additive, drink additive, animal feed, animal feed additive, animal feed supplement, dietary supplement, carrier, vitamin or mineral premix, nutritional product, enteral feeding product, soluble, slurry, supplement, pharmaceutical, lick block, drench, tablet, capsule, pellet or bolus, or wherein the L. rhamnosus FNZ129 is encapsulated, for example in liposomes, microbubbles, microparticles or microcapsules.

B25. The method of B24, wherein the L. rhamnosus FNZ129 or derivative thereof is administered in drinking water, milk, milk powder, milk replacement, milk fortifier, whey, whey powder, a feed pellet, corn, soybean, forage, grain, distiller's grain, sprouted grain, legumes, vitamins, amino acids, minerals, fibre, fodder, grass, hay, silage, kernel, leaves, meal, solubles, slurries, supplements, mash feed, meal, fruit pulp, vegetable pulp, fruit or vegetable pomace, citrus meal, wheat shorts, corn cob meal, molasses, sucrose, maltodextrin, rice hulls, vermiculite, zeolites or crushed limestone.

B26. The method of any one of B17 to B25, wherein the method comprises administering to the animal L. rhamnosus FNZ129 in an amount from 10⁴ to 10¹³ colony forming units per kilogram of dry weight carrier feed, from 10⁴ to 10¹⁰ colony forming units per kilogram of body weight of the animal per day, or from 10⁴ to 10¹³ colony forming units per day.

B27. The method of B26, wherein the method comprises administering to the animal L. rhamnosus FNZ129 in an amount from 10⁸ to 10¹² colony forming units per kilogram of dry weight carrier feed, from 10⁵ to 10⁸ colony forming units per kilogram of body weight of the animal per day, or from 10⁶ to 10¹³ colony forming units per day.

B28. The method of any one of B17 to B27, wherein the derivative of L. rhamnosus FNZ129 is a cell lysate of the strain, a cell suspension of the strain, a metabolite of the strain, a culture supernatant of the strain, or killed L. rhamnosus FNZ129.

B29. The method of any one of B17 to B28, the method comprising further administering at least one additional microorganism of a different species or strain, a vaccine that inhibits methanogens or methanogenesis, and/or a natural or chemically-synthesised inhibitor of methanogenesis and/or methanogen inhibitor such as bromoform.

B30. The method of any one of B17 to B29, wherein the L. rhamnosus FNZ129 or derivative thereof is administered separately, simultaneously or sequentially with one or more agents selected from one or more prebiotics, one or more probiotics, one or most postbiotics, one or more sources of dietary fibre, one or more galactooligosaccharides, one or more short chain galactooligosaccharides, one or more long chain galactooligosaccharides, one or more fructooligosaccharides, inulin, one or more galactans, one or more fructans, lactulose, or any mixture of any two or more thereof.

B31. The method of any one of B17 to B30, wherein the method additionally improves the body weight and/or body composition of the monogastric animal.

B32. The method of any one of B17 to B31, wherein said monogastric animal is a human, pig, cat, dog, horse, donkey, rabbit, or poultry.

B33. The method of any one of B17 to B31, wherein said monogastric animal is a companion animal.

B34. The method of any one of B17 to B31, wherein said monogastric animal is a non-human animal.

B35. The method of any one of B17 to B31, wherein said monogastric animal is a pig.

B36. The method of any one of B17 to B31, wherein said monogastric animal is a chicken, duck, goose or turkey.

B37. The method of any one of B17 to B35, wherein said monogastric animal is pre-weaning animal, for example a piglet or a foal.

B38. The method of any one of B17 to B35, wherein said monogastric animal is a post-weaning animal.

B39. The method of any one of B17 to B35, wherein the L. rhamnosus FNZ129 is administered to the monogastric animal both prior to weaning and after weaning.

B40. The method of any one of B17 to B35, wherein the administering is to a pre-weaning animal and wherein the inhibiting the growth of methane-producing bacteria and/or archaea in the gastrointestinal tract of monogastric animals, the reducing methane emissions, for example methane production, by the monogastric animal, and/or the increasing feed efficiency in the monogastric animal persists post-weaning.

B41. The method of any one of B17 to B35, wherein the inhibiting the growth of methane-producing bacteria and/or archaea in the gastrointestinal tract of monogastric animals, the reducing methane emissions, for example methane production, by the monogastric animal, and/or the increasing feed efficiency in the monogastric animal persists for at least 2 days, 3 days, 5 days, 1 week, 2 weeks, 1 month, 2 months, 3 months, 4 months, 5 months, 6 months, 7 months 8 months, 9 months, 10 months, 11 months, 1 year, 2 years, 3 years, 4 years, 5 years, 6 years, or 7 years after the last administration of L. rhamnosus FNZ129.

B42. The method of B41, wherein the inhibiting the growth of methane-producing bacteria and/or archaea in the gastrointestinal tract of monogastric animals, the reducing methane emissions, for example methane production, by the monogastric animal, and/or the increasing feed efficiency in the monogastric animal persists for the life of the monogastric animal.

B43. The method of any one of B17 to B42, wherein the L. rhamnosus FNZ129 is administered in a composition that is a fermented yoghurt-style composition, and wherein the fermented yoghurt-style composition is formed through a process of growing L. rhamnosus FNZ129 using a milk-based carrier.

B44. Use of Lacticaseibacillus rhamnosus (Lactobacillus rhamnosus) strain FNZ129, NMIA accession number V21/015446 dated 2 Aug. 2021, or a derivative thereof for the manufacture of a composition for inhibiting the growth of methane-producing bacteria and/or archaea in the gastrointestinal tract of monogastric animals, reducing the ability of the gastrointestinal microbiome to produce methane, reducing methane emissions by a monogastric animal, increasing feed efficiency in a monogastric animal, enhancing the growth and/or productivity in a monogastric animal, increasing the yield of milk and/or milk components produced from a monogastric animal, or improving the body weight and/or body composition of a monogastric animal.

B45. The use of B44, wherein the composition is a medicament.

B46. The use of B44 or B45, wherein the monogastric animal is a human.

B47. Lacticaseibacillus rhamnosus (Lactobacillus rhamnosus) strain FNZ129, NMIA accession number V21/015446 dated 2 Aug. 2021, or a derivative thereof for use in inhibiting the growth of methane-producing bacteria and/or archaea in the gastrointestinal tract of monogastric animals, reducing the ability of the gastrointestinal microbiome to produce methane, reducing methane emissions by a monogastric animal, increasing feed efficiency in a monogastric animal, enhancing the growth and/or productivity in a monogastric animal, increasing the yield of milk and/or milk components produced from a monogastric animal, or improving the body weight and/or body composition of a monogastric animal.

B48. The L. rhamnosus FNZ129 or derivative thereof for use of B47, wherein the monogastric animal is a human.

### EXAMPLES

### 1. Example 1—Plate-Based Screen of Bacteriocin Extracts Against Indicator Methanogen Strains

### 1.1 Materials and Methods

### 1.1.1 Bacteriocin Extraction

Bacteriocin extracts from L. rhamnosus FNZ129 cultures were prepared and tested for their effect against indicator methanogen strains Methanobrevibacter boviskoreani JH1 (‘JH1’), Methanosphaera sp. WGK6 (‘WGK6’), Methanobrevibacter ruminantium M1 (‘M1’) and Methanobrevibacter gottschalkii D5 (‘D5’).

L. rhamnosus FNZ129 was revived from −80° C. storage by plating onto De Man-Rogosa-Sharpe agar (MRS, De Man et al., 1960)+lactose (2 g/L). Using a small inoculating loop, glycerol stocks were streaked onto MRS agar plates to obtain an isolated colony. The plates were incubated for 48 h in a sealed container at 37° C. After growth, a single colony was selected, picked up and re-streaked a second time on an agar plate and incubated at 37° C. to obtain an isolated colony. After 48 hours, a single colony from the re-streaked plate was selected and inoculated into the MRS liquid medium, and incubated at the 37° C. for 48 h. An inoculum (1 mL) of each revived strain was then sub-cultured into 16 mL MRS+nisin liquid medium (1 ng/mL final conc.). The nisin was included in these media at a very low level to induce bacteriocin production. The cultures were incubated overnight at 37° C. Overnight-grown L. rhamnosus FNZ129 cultures were used for bacteriocin extraction. A drop of the culture was used to make a wet mount slide to examine cells using phase contrast microscopy and to prepare a Gram stain to check culture purity.

The remainder of the culture (˜16 mL) was transferred into a 50 mL Falcon tube and used for bacteriocin extraction following the method of Gaspar et al. (2018), with some modifications as follows. The pH of the culture was adjusted to ˜6.8 with 6M NaOH. Then 0.3 mL of catalase (2 mg/L) was added to the culture and incubated for 30 min at 37° C. followed by an incubation at 70° C. for 45 min. The cultures were then centrifuged for 20 min at 12,000×g at 4° C., the supernatant was decanted, and the cell pellet was resuspended in 8 mL 0.9% NaCl, pH 2. The pH of the resuspended pellet was checked and, if necessary, adjusted to pH 2 with 1M HCl. The cells were incubated for 2 hr at 4° C. with slow agitation on a shaking platform. The cells were then centrifuged at 12,000×g for 20 min at 4° C., and the supernatant collected into a fresh 15 mL Falcon tube. The pH of the supernatant was adjusted to pH 6.8 with 1M NaOH and filtered through a sterile filter (Millex-GP 0.22 μm, 25 mm diameter, Millipore, Merck, Sigma-Aldrich NZ) into a sterile, N₂-flushed, Hungate tube using a 10 mL syringe and needle under sterile conditions. The filtered supernatant was frozen at −20° C. until use.

1.1.2 Mbb. Boviskoreani JH1 Culture

To identify potential candidate LAB strains with anti-methanogen activities, a microtitre plate-based methanogen growth inhibition bioassay using the model methanogen strain, Methanobrevibacter boviskoreani JH1 (Li et al, 2019) was used. Mbb. boviskoreani JH1 has the unusual ability to grow using ethanol as a source of reducing power to reduce CO₂ to CH₄ allowing JH1 growth in microtitre plates incubated under anaerobic conditions without the need to supply H2 via a 1 atm overpressure of H₂:CO₂ (80:20). This allows a high throughput JH1 screening method to identify inhibitory activities from LAB strains.

The Mbb. boviskoreani JH1 cultures for inoculating the plate assays were grown in Balch tubes (Anaerobic tube, 18×150 mm, butyl rubber septum stopper, aluminium crimps, Bellco Glass, Vineland, NJ, USA) containing 9 mL BY medium (Joblin, 1995) supplemented with (final concentrations) 60 mM sodium formate, 200 mM ethanol, 0.1 mL of Vitamin Solution (1×) and 0.1 mL of Coenzyme M Solution (10 μM) by syringe using anaerobic techniques. The tubes were incubated at 39° C. without shaking until visible turbidity appeared after 3 to 5 days and were used for inoculation of the microtitre plate assays after they attained an OD₆₀₀ of between 0.8 to 1.0 against a distilled water blank. The over-pressure in the JH1 culture tubes was released by inserting a needle through the butyl rubber septum and allowing the accumulated gases to escape, prior to removing the inoculum.

The freshly grown cultures were checked using wet mounts under fluorescence microscopy, and Mbb. boviskoreani JH1 appeared as short ovoid-shaped rods that fluoresced green under ultraviolet (UV) illumination. Culture contamination was checked by inoculating a sample of the culture into 9 mL BY medium supplemented with 5 mM glucose and incubating at 39° C. for one day. If no turbidity was seen after 1 day, then the culture was considered uncontaminated. Further culture verification was conducted from time to time by extracting the genomic DNA from the culture and PCR amplifying the 16S rRNA gene, using both the conventional bacterial 16S primers (27f-GAGTTTGATCMTGGCTCAG, 1492r-GGYTACCTTGTTACGACTT) and the archaeal-specific 16S primers (915 af-AGGAATTGGCGGGGGAGCAC, 1386r-GCGGTGTGTGCAAGGAGC). The presence of a band with the archaeal primer set and the absence of a band with the bacterial primer set, and the sequencing results from the PCR products, were used to confirm culture purity.

1.1.3 Methanosphaera Sp. WGK6 Culture

Members of the genus Methanosphaera make up around 8% of rumen methanogens (Henderson et al., 2015) and are generally H2-dependent methylotrophs, using H2 to reduce methanol to methane. Methanosphaera sp. WGK6 is a H2-utilising methylotrophic methanogen isolated from the gut of a kangaroo in Australia, but it is also able to use ethanol as a source of reducing power to reduce methanol to methane (Hoedt, 2017).

Similar to Mbb. boviskoreani JH1, this metabolic capability theoretically allows WGK6 to grow on ethanol without the need for an over-pressure of H₂, and thus enable it to grow in a microtitre plate. The growth of Methanosphaera sp. WGK6 was tested using BRN-RF10 medium (Balch et al., 1979; Hoedt, 2017) in Hungate tubes with H₂ (180 kPa over-pressure of H₂+CO₂; 80:20) or ethanol as the energy sources and methanol as the terminal electron acceptor in both cases. Attempts to grow WGK6 on ethanol+methanol were unsuccessful, but WGK6 was able to grow on methanol+H₂ in Hungate tubes. Our initial attempts to grow Methanosphaera sp. WGK6 in a microtitre plate format with methanol under a H₂+CO₂ atmosphere (180 kPa over-pressure) in a pressurised gas cannister, produced barely detectable growth after 1 week. However, after increasing the concentration of cysteine added to the BRN-RF10 medium, better growth of Methanosphaera sp. WGK6 was obtained. So, a plate assay using stainless steel gas cannisters able to be pressurize (H₂+CO₂; 80:20) was developed.

The Methanosphaera sp. WGK6 cultures for the assay were grown in Balch tubes in 9 mL BRN-RF10 medium supplemented with (final concentrations) 60 mM sodium formate, 1% methanol, 0.1 mL of Vitamin Solution (1×) and 0.1 mL of Coenzyme M Solution (10 μM) by syringe using anaerobic techniques and with 180 kPa over-pressure of H₂+CO₂ (80:20, BOC Gases NZ). The tubes were incubated at 39° C. without shaking until visible turbidity appeared after 3 to 5 days and were used for inoculation of the tube assays after they attained an OD₆₀₀ of between 0.8 to 1.0 against a distilled water blank. The over-pressure in the WGK6 culture tubes was released by inserting a needle through the butyl rubber septum and allowing the accumulated gases to escape, prior to removing the inoculum.

1.1.4 Mbb. ruminantium M1 and Mbb. Gottschalkii D5 Culture

The procedure for growing Methanobrevibacter ruminantium M1 and Methanobrevibacter gottschalkii D5 was identical to the WGK6 protocol described in 1.1.3 above, except it used BY medium for growth. The cultures for the assays were grown in Balch tubes in 9 mL BY medium supplemented with (final concentrations) 60 mM sodium formate, 0.1 mL of Vitamin Solution (1×) and 0.1 mL of Coenzyme M Solution (10 μM) added by syringe using anaerobic techniques and with 180 kPa over-pressure of H₂+CO₂ (80:20, BOC Gases NZ). The tubes were incubated at 39° C. without shaking until visible turbidity appeared after 3 to 5 days and were used for inoculation of the microtitre plate assays.

1.1.5 Mbb. Boviskoreani JH1 Growth Inhibition Assay

The bacteriocin extracts from L. rhamnosus FNZ129 stored frozen under anaerobic conditions in Hungate tubes were allowed to thaw at room temperature. All of the assay components for each assay, except the JH1 inoculum, were added via CO₂-flushed syringes and needles to 3.75 mL BY+formate medium in sterile 7.5 mL Hungate tubes in the proportions indicated in Table 1. Each tube was then inoculated with freshly grown JH1 culture, incubated for 1 hr at 39° C., then moved inside an anaerobic chamber (98% CO₂-2% H₂ atmosphere; Coy Laboratory Products, USA) and dispensed into wells of multiwell 96 well plates. The filled plates were placed into an AnaeroPack 2.5 L Rectangular Jar with an MCG Anaeropack-Anaero (Ngaio Diagnostics, Nelson, NZ), the lid sealed, and the jar removed from the anaerobic chamber and incubated at 39° C. The plate was observed daily through the transparent jar, until the Mbb. boviskoreani JH1 control wells showed visible turbidity (usually within 5 to 6 days). The optical density of each well was then recorded at 595 nm (OD₅₉₅) after 5 seconds shaking in a Multiskan F C Microplate Photometer (Thermo Scientific, Auckland, NZ). The absorbance readings of the media control wells were subtracted as background, and the % inhibition of Mbb. boviskoreani JH1 growth caused by the bacteriocin extract samples, relative to the JH1 positive growth control wells (which contained buffer alone) was calculated.

TABLE 1
Microtitre plate setup for the Mbb. boviskoreani
JH1 growth inhibition assay.

| | Amount added (μl) | | | |
|---|---|---|---|---|
| | LAB | | JH1 | |
| | bacteriocin | Media | alone | |
| Component | extract | control | control | Nisin |
| BY medium + | 150 | 150 | 150 | 150 |
| formate (3M) | | | | |
| Phosphate | 5 | 90 | 80 | 70 |
| buffer (1M) | | | | |
| Ethanol (10M) | 5 | 5 | 5 | 5 |
| Vitamin/CoM | 5 | 5 | 5 | 5 |
| solution (1×) | | | | |
| Bacteriocin | 75 | 0 | 0 | 0 |
| extract | | | | |
| Nisin | 0 | 0 | 0 | 10 |
| (1 mg/ml, 300 | | | | |
| μM) | | | | |
| JH1 inoculum | 10 | 0 | 10 | 10 |
| TOTAL | 250 | 250 | 250 | 250 |

1.1.6 Methanosphaera sp. WGK6 Growth Inhibition Assay Each of the assay components for the assay, except the WGK6 inoculum, were added via CO₂-flushed syringes and needles to 3.75 mL BRN-RF10 medium in Hungate tubes supplemented with 1% methanol (247 mM, final conc.) as described in Table 4. The tubes were then moved into the chamber, along with the inoculum tube. The medium containing all the components except the inoculum were dispensed into the plates inside the chamber, and then the inoculum was added to the appropriate wells. The plates were placed into a stainless-steel gas cannister laid horizontally, to hold up to 4 microtitre plates at a time. Two anaerobic sachets (MCG Anaeropack-Anaero, Ngaio Diagnostics, Nelson, NZ) were added, the cannister was sealed and the cannister was taken out of the anaerobic chamber and pumped to a pressure 180 kPa with H₂+CO₂ (80:20, BOC Gases NZ), then incubated at 39° C. for 1 week. The cannisters were checked periodically to ensure an over-pressure was maintained, and if necessary, re-pressurised with H₂+CO₂. After incubation for 1 week, the cannister was opened and the plates were removed. The contents of each well were resuspended evenly by repeated pipetting with a multichannel pipettor. The optical density of each well was then immediately recorded at 595 nm (OD₅₉₅) after 5 seconds shaking in a Multiskan FC Microplate Photometer (Thermo Scientific, Auckland, NZ). The absorbance readings of the media control wells were subtracted as background, and the % inhibition of Methanosphaera sp. WGK6 growth caused by the bacteriocin extract samples, relative to the WGK6 positive growth control wells (which contained buffer alone in place of bacteriocin extract) was calculated.

TABLE 2
Microtitre plate setup for the Methanosphaera sp.
WGK6 growth inhibition assay.

| | Amount added (ml) | | | |
|---|---|---|---|---|
| | Media | Nisin | Growth | Bacteriocin |
| Component | control | control | control | test |
| BRN-10 Media | 3.5 | 3.5 | 3.5 | 3.5 |
| 3M Sodium | 0.1 | 0.1 | 0.1 | 0.1 |
| Formate, 1M | | | | |
| Sodium | | | | |
| Acetate, 1M | | | | |
| Methanol mix | | | | |
| 25 mg/ml L- | 0.1 | 0.1 | 0.1 | 0.1 |
| cysteine-HCl | | | | |
| Methanogen | 0 | 0.5 | 0.5 | 0.5 |
| inoculum | | | | |
| Nisin (1 mg/ | 0 | 0.2 | 0 | 0 |
| mL, 300 μM) | | | | |
| Bacteriocin | 0 | 0 | 0 | 0.8 |
| extract | | | | |
| 0.9% NaCl | 0.8 | 0.6 | 0.8 | 0 |
| Additional | 0.5 | 0 | 0 | 0 |
| BRN-10 (in | | | | |
| place of | | | | |
| inoculum) | | | | |
| TOTAL | 5.0 | 5.0 | 5.0 | 5.0 |

1.1.7 Mbb. ruminantium M1 and Mbb. gottschalkii D5 Growth Inhibition Assays

Cultures of Mbb. ruminantium M1 and Mbb. gottschalkii D5 were prepared as described in 1.1.4 above. The over-pressure in the tubes was released prior to removing the inoculum.

The assay components were added to 3.5 mL of sterile BY medium in a 7.5 mL Hungate tube via CO₂-flushed syringes and needles as describe in Table 3. Each tube was then inoculated with freshly-grown culture, incubated for 1 hr at 39° C., then moved inside the anaerobic chamber and dispensed into wells of 96-well multiwell plates. The plates were sealed and incubated in stainless steel gas cannisters under 180 kPa over-pressure of H₂+CO₂ and their optical densities recorded by spectrophotometric measurement at OD₅₉₅ as described for the Methanosphaera sp. WGK6 assay in 1.1.6 above. The OD₅₉₅ readings of the BY media control wells were subtracted as background, and the % inhibition of the growth of the Mbb. ruminantium M1 or Mbb. gottschalkii D5 caused by bacteriocin extract samples, relative to the positive growth control wells (which contained buffer in place of the bacteriocin extract) were calculated.

TABLE 3
Microtitre plate setup for the Mbb, ruminantium
M1 and Mbb. gottschalkii D5 growth inhibition assays.

| | Amount added (ml) | | | |
|---|---|---|---|---|
| | Media | | Growth | Bacteriocin |
| Component | control | Nisin | control | test |
| BY medium | 7.0 | 7.0 | 7.0 | 3.5 |
| 3M sodium | | | | |
| formate/1M | 0.2 | 0.2 | 0.2 | 0.1 |
| sodium | | | | |
| acetate mix/ | | | | |
| 1M methanol | | | | |
| Vitamin/CoM | 0.2 | 0.2 | 0.2 | 0.1 |
| solution (1×) | | | | |
| 25 mg/ml L- | 0.2 | 0.2 | 0.2 | 0.1 |
| cysteine-HCl | | | | |
| Methanogen | 0 | 1.0 | 1.0 | 0.5 |
| inoculum (M1 | | | | |
| or D5) | | | | |
| Nisin (1 mg/ | 0 | 0.4 | 0 | 0 |
| mL, 300 μM) | | | | |
| Bacteriocin | 0 | 0 | 0 | 0.8 |
| extract | | | | |
| Additional BY | 1.0 | 0 | 0 | 0 |
| medium | | | | |
| NaCl 0.9% | 1.6 | 1.2 | 1.6 | 0 |
| TOTAL | 10.2 | 10.2 | 10.2 | 5.1 |

### 1.2 Results

Bacteriocin extracts from a total of 1,712 strains of lactic acid bacteria were screened against Methanosphaera sp. WGK6. Of these, 1,580 strains (>92%) showed less than 50% inhibition. The 1,712 strains of lactic acid bacteria included 94 strains of Lacticaseibacillus rhamnosus, of which 62 (˜66%) showed less than 20% inhibition of WGK6, 81 (˜86%) showed less than 50% inhibition, and only 3 strains (˜3%) showed >80% inhibition. Together, this indicates that methanogen inhibition is likely to be a strain-specific effect.

The L. rhamnosus FNZ129 bacteriocin extract showed very strong inhibition of the indicator methylotrophic methanogen Methanosphaera sp. WGK6, but no inhibition of indicator hydrogenotrophic methanogens Mbb. boviskoreani 31H1, Mbb. ruminantium M1, or Mbb. gottschalkii D5, as shown in Table 4.

TABLE 4
Inhibition of indicator methanogen strains by
L. rhamnosus FNZ129 bacteriocin extract.

| | % inhibition | | | |
|---|---|---|---|---|
| | JH1 | WGK6 | M1 | D5 |
| FNZ129 | 0 | 94 | 0 | 2 |

### 1.3 Discussion and Conclusion

Members of the Methanobrevibacter and Methanosphaera genera are the predominant methanogens in the rumen across multiple ruminant species. WGK6 was used as an indicator strain for methylotrphic methanogens in general and Methanosphaera spp. in particular. Mbb. boviskoreani JH1, Mbb. ruminantium M1, and Mbb. gottschalkii D5 were used as indicator strains for Methanobrevibacter spp. This Example shows that L. rhamnosus FNZ129 bacteriocin extract shows a strong inhibitory effect against the methylotrophic methanogen Methanosphaera sp. WGK6, but no effect against the hydrogenotrophic Mbb. boviskoreani JH1, Mbb. ruminantium M1, and Mbb. gottschalkii D5 methanogens.

### 2. Example 2—Impact of L. rhamnosus FNZ129 on Rumen In Vitro Assays

### 2.1 Materials and Methods

2.1.1 Preparation of bacterial cultures and supernatants for testing

L. rhamnosus FNZ129 was used to inoculate 7 Hungate tubes, each containing 5 mL of anaerobic MRS medium (Sigma-Aldrich), and these were incubated at 39° C. for 16 hours (until the cultures reached stationary phase). Cultures were pooled into a 250 mL CO₂-flushed serum bottle. An aliquot (1 mL) of the combined cultures was added to 9 mL of sterile MRS medium to measure its OD₆₀₀. Further aliquots (0.5 mL) of the culture mix were inoculated in triplicate into 4.5 mL of sterile anaerobic buffer and serially 10-fold diluted under CO₂ and plated onto MRS plates to determine the number of colony forming units (CFU·mL⁻¹) of original culture. Half of the remaining culture was used for one set of rumen in vitro fermentations (test culture) and the other half was filtered (Millipore 0.22 μm pore size) and the filtrate was placed into a new sterile anaerobic serum bottle (supernatant treatment, SN). Anaerobic phosphate buffer (0.46 M K₂HPO₄; 0.54 M KH₂PO₄, pH 7) was used as the no treatment control (Buffer).

### 2.1.2 Rumen Fluid Preparation and In Vitro Fermentation Set Up

For inoculation of the rumen in vitro fermentation vessels, fresh rumen contents were collected from 6 rumen-fistulated Friesian cows. After squeezing through 1 layer of cheesecloth, the resulting rumen fluids from two animals were combined (approx. 150 mL rumen fluid) giving 3 biological replicates. Aliquots (12.5 mL) of the mixed rumen fluid were added to 0.5 mg dried grass and 36.5 mL of anaerobic phosphate buffer in a 250 mL serum bottle. The treatments (1 mL) of either Buffer, test culture, SN or bacteriocin extract were added before closing the serum bottles with butyl rubber stoppers, giving a final fermentation volume of 50 mL containing 25% rumen fluid (v/v). Gas production and methane content was measured using an automated incubation system (Muetzel et al., 2014).

### 2.1.1 VFA Sample Collections and Analysis

Samples were collected from bottles for VFA analysis. At each time point, 3 mL aliquots were collected, and their pH measured. 1.8 mL samples of these aliquots were used for VFA and non-VFA analyses. VFA samples were centrifuged at 21,000×g for 10 min at 4° C. and 0.9 mL of supernatant was removed and added to 0.1 mL of internal standard (20 mM 2-ethylbutyrate in 20% phosphoric acid), mixed and frozen at −20° C. until analysis. After thawing and re-centrifugation at 21,000×g for 10 min at 4° C., 0.9 mL was collected for derivatization for non-VFA analysis, while the remainder of the sample was analysed directly via GC.

### 2.2 Results

L. rhamnosus FNZ129 was tested for its impact on gas production in rumen in vitro assays, as shown in Tables 5 to 10. The data shown are averages of three replicates. Negative numbers represent stimulation, rather than inhibition. An asterisk (*) is used to indicate statistical significance (p<0.05) by Student's T test with Welch's correction.

TABLE 5
Percent inhibition of total gas produced
(ml per g of substrate) compared to control.

| | 2 hours | 6 hours | 12 hours | 20 hours |
|---|---|---|---|---|
| FNZ129 | 16.8 | 9.3 | 9.1 | 5.4 |
| Supernatant | 8.2 | −0.3 | −1.6 | −0.7 |
| Extract | −0.7 | −0.9 | 0.9 | 0.9 |

TABLE 6
Percent inhibition of total methane produced
(ml per g of substrate) compared to control.

| | 2 hours | 6 hours | 12 hours | 20 hours |
|---|---|---|---|---|
| FNZ129 | 37.0 * | 11.94 * | 8.1 | 3.2 |
| Supernatant | 21.3 * | 6.6 * | 2.1 | 1.0 |
| Extract | 4.4 | 1.6 | 2.6 | 2.3 |

TABLE 7
Percent inhibition of total volatile fatty acids
produced (mM) compared to control.

| | 0 hours | 6 hours | 12 hours | 24 hours |
|---|---|---|---|---|
| FNZ129 | 1.1 | 5.9 | −12.5 | 4.2 |
| Supernatant | 3.4 | −1.3 | −14.9 | 6.0 |
| Extract | 6.4 | −20.5 * | −6.8 | −8.2 |

TABLE 8
Percent inhibition of acetic acid
produced (mM) compared to control.

| | 0 hours | 6 hours | 12 hours | 24 hours |
|---|---|---|---|---|
| FNZ129 | 2.7 | 4.8 | −13.4 | 3.6 |
| Supernatant | 4.2 | 2.3 | −3.4 | 7.5 |
| Extract | 10.9 | −29.4 * | −10.8 | −1.6 |

TABLE 9
Percent inhibition of propionic acid
produced (mM) compared to control.

| | 0 hours | 6 hours | 12 hours | 24 hours |
|---|---|---|---|---|
| FNZ129 | 1.4 | 7.6 | −12.4 | 4.4 |
| Supernatant | 2.8 | 2.2 | −16.0 | −1.5 |
| Extract | 12.0 | −15.1 | −10.0 | −9.3 |

TABLE 10
Percent inhibition of butyric acid
produced (mM) compared to control.

| | 0 hours | 6 hours | 12 hours | 24 hours |
|---|---|---|---|---|
| FNZ129 | 2.4 | 5.1 | −7.0 | 19.4 |
| Supernatant | −5.0 | 7.7 | −16.4 | −2.8 |
| Extract | 8.9 | −30.2 * | −9.3 | −8.0 |

The addition of L. rhamnosus FNZ129 broth culture caused a decrease in the total gas produced, particularly at 2 h, while culture supernatant and extract showed little or no effect on total gas produced. However, broth culture and culture supernatant both caused a significant decrease in the total amount of methane produced at 2 and 6 hours.

L. rhamnosus FNZ129 broth culture and culture supernatant showed little effect on the volatile fatty acids produced, however the bacteriocin extract significantly stimulated the overall production of volatile fatty acids at 6 hours. This effect can also be seen as a significant stimulation of the production of acetic and butyric acids at 6 hours, and a smaller stimulation of propionic acid production can also be seen at 6 hours.

It should also be noted that the rumen in vitro assays are a closed system and may become nutrient-limited over time. Therefore, the 0-12 hour timepoints may more accurately reflect the situation in vivo, as animals will typically ingest more food and liquid over a 24-hour period.

### 2.3 Conclusion

This Example shows that L. rhamnosus FNZ129 demonstrated significant impacts on fermentation end products. Overall, the results show that L. rhamnosus FNZ129 and culture supernatant thereof significantly reduced the total amount of methane produced in a rumen in vitro model. This may be mediated by a compound or compounds secreted by the bacteria into the culture supernatant. It also shows that a bacteriocin extract from FNZ129 has a significant stimulatory effect on the production of volatile fatty acids, in particular acetic and butyric acid. This suggests a shift in hydrogen metabolism away from methanogenesis to short chain/volatile fatty acid (VFA) production and/or disruption of cross-feeding of intermediates between members of the microbiome due to changes in the rumen microbiome. The significant increases in total VFA and butyric acid, and increase in propionic acid, suggests that animal feed efficiency is also likely to be improved.

### 3. Example 3—the Effect of L. rhamnosus FNZ129 on Piglet Weight

### 3.1 Materials and Methods

The experimental protocol for a pig probiotic trial using freeze dry probiotic product (Fonterra) was approved by the AgResearch Grasslands Animal Care and Ethics Committee (approval number 15323).

A total of 16 piglets were enrolled in the trial at 3 days of age. The piglets were weighed and assigned at random to one of the 2 treatment groups (n=8): FNZ129, receiving 5×10¹⁰ CFU/d of the probiotic organism Lacticaseibacillus rhamnosus FNZ129; and the Control group, without LAB feeding. The piglets were placed individually into custom cages constructed to allow animals to see, hear, and smell adjacent piglets while minimising physical contact (as described by Fil et al., 2021), each fitted with a heating-pad, an automatic milk feeder and free access to water via a water bowl. The piglets remained in these cages except during the time when the cages were being cleaned when they were kept in a large communal open pen where they could interact and play for at least 2 h/d. During the first 5 weeks, the piglets were fed exclusively on milk (reconstituted from milk powder). To feed the piglets, the amount of milk required for 8 piglets was prepared and 1 sachet of freeze dried (FD) FNZ129 containing the required dose for 8 animals was added to the milk. The milk was placed into the automatic milk feeder connected to an electronic system allowing regular dispensing of milk over a 24 h period. Frozen ice packs were placed around the feeder reservoirs to keep the milk cool and prevent microbial growth. Pigs were weighed every third day.

At 5 weeks of age, solid food was introduced into the diet in the form of Little Pig Tucker pellets (NRM Feeds, NZ), and the piglets were weaned by slowly decreasing the amount of milk offered, (keeping the probiotic dose constant) until week 8 when only pellets were offered twice daily (morning; afternoon). Water was freely available until the end of the trial. During this period at 7 weeks of age the piglets (˜20 kg Live weight; LWT) were moved to larger pens in a covered barn. Each pen had a raised wooden sleeping area with a heat pad, a feed trough and water available via a self-activated nipple. Once the piglets had transitioned completely to the pellet diet, the amount of FNZ129 required for 8 pigs was reconstituted in a small amount of water (200 mL) and mixed evenly into ˜1.6 kg of pellets. The pellets containing the FNZ129 were then evenly divided into 8 aliquots (200 g/pig) and fed to the appropriate pigs. The Control animals received the same quantity of pellets treated with water only. These pellet+treatment mixtures were provided as the first feed in the morning when pigs were hungry to ensure the entire LAB dose was consumed each day. Once the pellet+treatment mixtures were eaten, the main meal of dry pellets was topped up for the morning feed. The pigs were fed ad libitum pellets from 8 weeks of age until the end of the trial at 19 weeks.

The piglets suffered an episode of rotavirus infection during Week 4 of the trial and the animals were given electrolyte therapy and Scourban Plus (Bayer NZ). A second round of rotavirus infection occurred at Week 9 of the trial after moving the pigs to the larger pens. The pigs were again treated with electrolyte therapy and all animals recovered well.

### 3.2 Results

This Example showed that supplementation with L. rhamnosus FNZ129 did not adversely affect pig weight (Table 11).

TABLE 11
Effect of bacterial strains on pig weight.
Values indicate mean weight ± standard deviation.

| | Average pig weight (kg) | | |
|---|---|---|---|
| | Control | | Student |
| | treatment | FNZ129 | T test |
| Age (days) | (n = 8) | (n = 8) | p value |
| 49 | 17.7 ± 1.2 | 17.4 ± 0.7 | 0.65 |
| 53 | 20.8 ± 1 | 20.7 ± 1.2 | 0.88 |
| 55 | 21.9 ± 1 | 21.4 ± 1.5 | 0.47 |
| 59 | 25.2 ± 1.4 | 24.6 ± 1.3 | 0.48 |
| 62 | 27.8 ± 1.8 | 26.9 ± 1.5 | 0.35 |
| 73 | 35.2 ± 2.3 | 32.6 ± 2.4 | 0.07 |
| 76 | 38.5 ± 1.7 | 35.6 ± 2.4 | 0.051 |
| 80 | 41.9 ± 2.3 | 39.8 ± 3.2 | 0.20 |
| 82 | 45.3 ± 3.8 | 43.1 ± 3 | 0.28 |
| 87 | 49.8 ± 3.5 | 47.1 ± 4.7 | 0.26 |
| 90 | 53.8 ± 3 | 50.8 ± 4.4 | 0.17 |
| 94 | 56 ± 3.1 | 53.4 ± 4.2 | 0.23 |
| 98 | 63.3 ± 4.2 | 60.6 ± 4.7 | 0.31 |
| 104 | 73.5 ± 5.1 | 69.8 ± 7.4 | 0.31 |
| 111 | 82.8 ± 4.6 | 79.1 ± 8 | 0.31 |
| 122 | 100.1 ± 5.2 | 94.1 ± 9.1 | 0.17 |

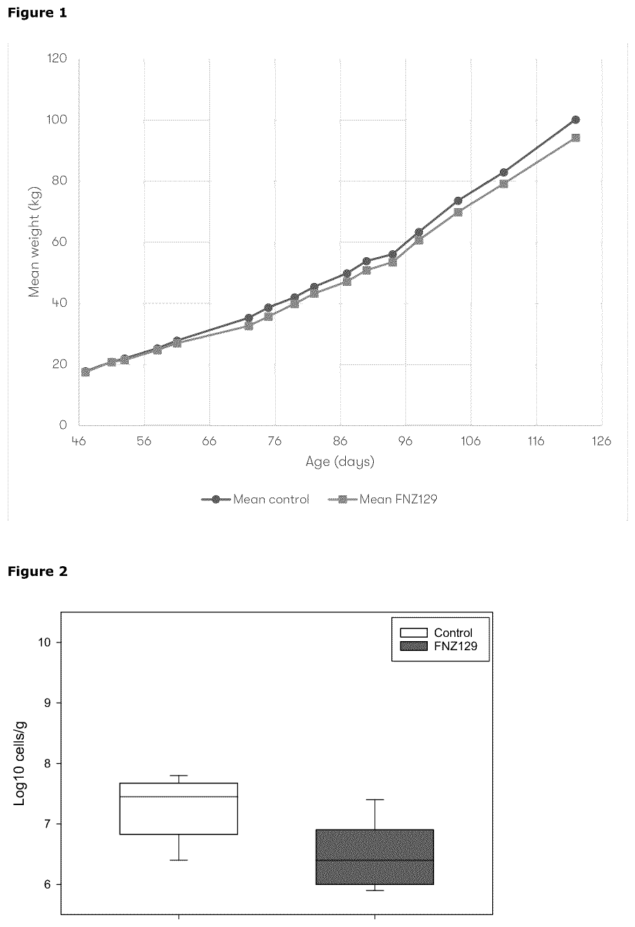

Table 11 and FIG. 1 show that supplementation with the bacterial strain FNZ129 did not adversely affect pig weight gain.

### 3.3 Conclusion

Supplementation of feed with L. rhamnosus FNZ129 did not have a significant negative effect on pig weight. This work indicates that supplementation with FNZ129 can reduce methane emissions without adversely affecting weight gain.

### 4. Example 4—Effect of L. rhamnosus FNZ129 on Number of Methanogenic Bacteria in Pigs

### 4.1 Materials and Methods

### 4.1.1 Animal Trial: Experimental Design and Animal Ethics Approval

The piglets used in Example 3 were used for this trial.

### 4.1.2 Pig Gut Sample Collection

At 19 weeks of age, the pigs were euthanized (captive bolt stunning, weighed, followed by exsanguination), and their caecum and colorectal regions were tied off and removed to collect their digestive contents from these two gut regions. The gut contents samples were used for enumeration of methanogens using a Most Probable Number (MPN) method, and the remaining samples were for volatile fatty acid (VFA) analyses by Gas Chromatography. For the MPN analysis, 5 mL Eppendorfs were filled with caecal or colorectal contents for each animal and placed on ice until further processing in the laboratory. For VFA analysis, caecal contents were aliquoted into 50 mL Falcon tubes, while colorectal contents were sampled into 15 mL Falcon tubes, and placed immediately on ice prior to storage at −20° C.

### 4.1.3 Most Probable Number (MPN)

The MPN method (McCrady, 1918) was used to estimate the number of microorganisms able to produce methane from samples of caecal and colorectal contents. Briefly, measured amounts of sample (approximatively 1 g), were added into the first RM02 (Kenters et al. 2011) dilution tubes and the weights were recorded for use in the final calculations. From the first dilution tube, 10-fold serial dilutions (1 mL into 9 mL RM02 medium) were performed. Each dilution was mixed evenly, then further diluted a total of 10 times, under anaerobic conditions. Dilutions were selected from the series and samples were inoculated into Balch tubes containing BRN-RF10 medium (Balch et al., 1979; Hoedt, 2017) supplemented with methanol (100 mM final conc.) and pressurized with 180 kPa overpressure of H₂+CO₂. The inoculated BRN-RF10 medium tubes were incubated horizontally at 39° C. for 1 month. These conditions allow the growth of hydrogenotrophic organisms such as the methanogenic archaea and homoacetogens, as well as methylotrophic methanogens. After 1 month, the tubes were placed at room temperature for 30 min before carrying out headspace gas analysis using gas chromatography. Gas samples (0.5 mL) were collected from the tube headspace at the pressure in the culture vessel using polycarbonate 1 mL Luer-Lok syringes (Becton Dickinson and Co., Franklin Lakes, NJ, USA) fitted with Mininert Luer-tip syringe valves (Hamilton, Reno, NV, USA). The headspace gas samples were manually injected into an Aerograph 660 gas chromatograph (Varian Associates, Palo Alto, CA, USA) fitted with a Porapak Q80/100 mesh column (Waters Corporation, Milford, MA, USA) and a thermal conductivity detector. N₂ was used as the carrier gas. A 0.5 mL sample of gas standard containing H₂:CH₄:N₂ (5%:30%:65% v/v; BOC Gas, Palmerston North, NZ) was measured at 1 atm and used for calibration. The presence of methane was used as an indicator of methanogenic activity in the tube and was considered positive. If no methane was detected in the headspace, the tubes was considered as negative. By measuring the presence or absence of methane into the gaseous head space of the culture tubes, it is possible to determine in which tubes methanogens are present and metabolically active. From this data, and using MPN tables, the total number of methane-producing organism present in the original sample was calculated.

### 4.1.4 Media Preparation

RM02 was prepared anaerobically and dispensed under anaerobic conditions into Hungate tubes (9 mL per tube), then autoclaved for 20 min at 125° C. BRN-RF10 medium supplemented with (final concentrations) 60 mM sodium formate was prepared and dispensed under anaerobic condition into Balch tubes 9.8 mL per tube), then autoclaved for 20 min at 125° C. Before inoculation, 0.5% methanol (100 mM final), and 0.1 mL of Coenzyme M Solution (10 μM) were added by syringe using anaerobic techniques. After inoculation, the tubes were pressurised with 180 kPa over-pressure of H₂+CO₂ (80:20, BOC Gases NZ).

### 4.2 Results

TABLE 12
Most probable number (MPN) of methanogenic
bacteria per gram of pig faeces

| | Caecum | | Colorectal | |
|---|---|---|---|---|
| | MPN | fold change | MPN | fold change |
| Control | 2.9 × 10⁷ | N/A | 4.8 × 10⁸ | N/A |
| FNZ129 | 6.8 × 10⁶* | 4.23 | 2.6 × 10⁸ | 1.86 |

*Statistically significant, P = 0.015.

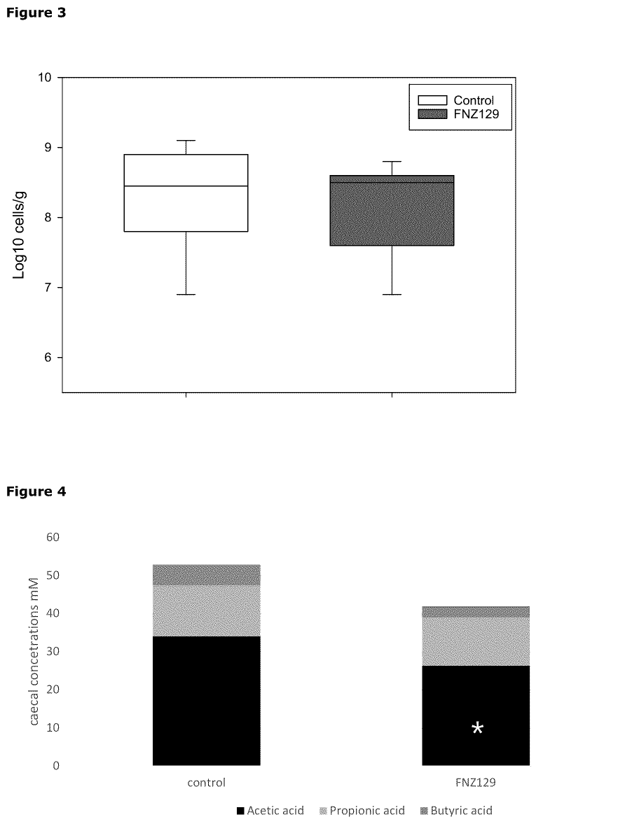

The results showed a significant decrease in methanogen numbers and methanogenic activity in the caecum of pigs treated with FNZ129 (Table 12, and FIGS. 2 and 3). The FNZ129 treatment also resulted in numerically lower methanogen numbers and methanogenic activity in the colorectal samples, although the difference did not reach statistical significance.

### 4.3 Discussion

The pig intestinal microbiome shares many similarities with that of humans, including the dominance of the two major phyla the Bacteroidetes and Firmicutes, which occur in similar proportions in both species. While relative abundances may vary, bacteria associated with human health, such as Lacticaseibacillus, Bifidobacterium, and Faecalibacterium are also commonly found in the pig. These similarities in the microbiome are likely to be a driven by the similarity in the digestive systems, which lead to common ecological constraints.

The MPN method allowed us to identify a significant impact of FNZ129 on caecal methanogens. The feeding of the FNZ129 strain decreased the population level of methane-producing organisms in the pig caecum, supporting the hypothesis that the strain could produce biological compound(s) inhibiting the growth of methanogens.

Independently of treatment, the lower methanogen populations in the pig caecum compared to the colorectum was expected as methanogens in monogastric animals prefer the terminal part of the digestive tract. Methanogen communities, as estimated by copy numbers of 16S rRNA genes, range from ˜10⁸ to 10⁹ organisms per gram of digestive contents from caecum to rectum. Similarly, methane percentage in gases produced from the gut range from 1.7-2.5% to 29-38%, from the caecum to the rectum, respectively. An increasing gradient of methanogen populations and methane formation in the pig hindgut from the caecum to the rectum exists (Jorgensen et 2011, Gresse et al 2019) and reflects slower transit times, more anaerobic conditions, and higher pH from the caecum to the colorectum. These conditions favour methanogens, allowing them to replicate and maintain their populations more easily.

Probiotics administered to pigs will first encounter methanogens after they pass through the stomach and small intestine and enter the caecum. Because methanogen numbers and activity are lower in the caecum, the effect of the probiotic strains are likely to have their greatest anti-methanogen effect in this gut compartment. Moreover, it is also expected to see the impact of probiotics on microbial fermentation through altered VFA profiles in this region of the gut as the caecum represents a preferred niche for lactobacilli. The LAB are likely be most active in this region and interact with other member of the ecosystem, or produce and/or release compounds into the caecum, to have their inhibitory effect on methanogen populations.

### 4.4 Conclusion

This Example shows that feed supplementation with L. rhamnosus FNZ129 can reduce the number of methanogenic microorganisms in the gut of pigs.

### 5. Example 5—Effect of Bacterial Strains on Volatile Fatty Acid Production in Pigs

### 5.1 Materials and Methods

### 5.1.1 Sample Collection

The pigs used in Examples 3 and 4 were used in this experiment. Directly after euthanasia, the pig caecum and colorectum regions of the gut were tied off and removed. Caecal contents were aliquoted into 50 mL Falcon tubes, while colorectal contents were sampled into 15 mL Falcon tubes, and placed immediately onto ice to be transferred into a −20° C. freezer until further analysis.

### 5.1.2 Sample Preparation for Gas Chromatography Analysis of VFAs

Samples were defrosted at room temperature. An aliquot (0.5 mL) of each sample was first removed. The remaining sample volume was centrifuged at 21,000×g for 10 min at 4° C. and 0.9 mL of the supernatant was removed and added to 0.1 mL of internal standard (20 mM 2-ethylbutyrate in 20% phosphoric acid), mixed and frozen at −20° C. until analysis. After thawing and re-centrifugation at 21,000×g for 10 min at 4° C., 0.2 mL of the supernatant was collected for derivatization for non-VFA analysis using gas chromatography (Richardson et al. 1989), while the remainder of the sample was analysed directly via gas chromatography (Attwood et al., 1998) using a gas chromatograph (Model 6869, Hewlett-Packard, Montreal, QC, Canada) equipped with an auto-sampler, and fitted with a Zebron ZB-FFAP 30.0 m×0.53 mm I.D.×1 μm film column (Phenomenex, Torrance, CA, United States) and a flame ionization detector set at 265° C.

### 5.2 Results

The main VFAs present in the caecal and colorectal sample were acetic, propionic, and butyric acids. Their respective concentrations and proportions correspond to normal levels of VFA present in monogastric animals.

### 5.2.1 Caecal VFAs

Significant differences were observed in caecal VFA concentrations between pigs fed with FNZ129 compared to control pigs. Caecal VFAs are reported as both absolute concentration of acetic, propionic, and butyric acid (FIG. 4) and the relative proportion of each of these as a percentage of total VFAs (Table 13). Administration of the FNZ129 probiotic strain significantly decreased the absolute acetic acid concentration (FIG. 4), and significantly increased the proportion of propionic acid relative to total VFAs (Table 13). Caecal concentrations of lactic acid and butyric acid in pigs fed FNZ129 were not significantly different from controls. No succinic acid was detected in any of the caecal contents and although total VFA concentrations were lower in FNZ129-treated animals (42.6 mM) compared to control animals (53.6 mM), this difference was not significant.

TABLE 13
Main volatile fatty acid percentages in pig
caecal samples collected after euthanasia.
n = 8 animals for control, n = 7 animals for FNZ129.

| | % Acetic acid | % Propionic acid | % Butyric acid |
|---|---|---|---|
| Control | 64 ± 3 | 24 ± 3 | 10 ± 3 |
| FNZ129 | 62 ± 3 | 30 ± 2 ** | 7 ± 2 |

** T test FNZ129 vs control, p < 0.01.

### 5.2.2 Colorectal VFAs

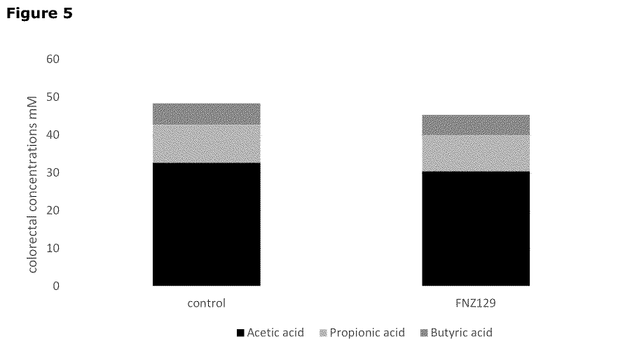

Colorectal VFAs are also reported as both absolute concentration of acetic, propionic, and butyric acid (FIG. 5) and the relative proportion of each of these as a percentage of total VFAs (Table 14). The results indicated that there was no impact of L. rhamnosus FNZ129 on the colorectal fermentation process (Table 14 and FIG. 5). The proportion of acetic acid in colorectal samples was higher than the control, however the difference was not significant.

TABLE 14
Main volatile fatty acid percentages in
pig colorectal samples collected after euthanasia.
n = 8 animals for control, n = 7 animals for FNZ129.

| | % Acetic acid | % Propionic acid | % Butyric acid |
|---|---|---|---|
| Control | 55 ± 7 | 17 ± 1 | 9 ± 2 |
| FNZ129 | 57 ± 6 | 18 ± 3 | 10 ± 2 |

### 5.3 Discussion

The MPN (Example 4) and VFA (Example 5) results demonstrate an impact of FNZ129 on the caecal fermentation process. Administration of FNZ129 decreased the absolute concentration of acetic acid and increased propionic acid as a percentage of total VFAs in the caecum. The impact of the FNZ129 strain was only seen in the caecum with no differences observed of FNZ129 treatment in the colorectal samples compared with the control group.

A change in the percentage of propionic acid could indicate a modification of hydrogen metabolism in the caecum. An increase in hydrogen accumulation could occur if methanogenic activity in the caecum was decreased, and increased hydrogen concentrations in the caecum would cause the thermodynamically favoured production of propionic acid. The differences observed in caecal acetic and propionic acids, combined with the lower methanogenic MPN result suggest that the FNZ129 strain has had an inhibitory effect on the methanogenic community in the caecum and/or disrupted cross-feeding of intermediates between members of the microbiome due to changes in the gastrointestinal microbiome, and potentially decreased their production of methane.

### 5.4 Conclusion

This Example shows that feed supplementation with L. rhamnosus FNZ129 significantly increased the proportion of propionic acid, suggesting altered hydrogen metabolism consistent with an inhibitory effect on caecal methanogens.

### REFERENCES

Anderson, R. C., Cookson, A. L., McNabb, W. C., Kelly, W. J., Roy, N. C., 2010. Lactobacillus plantarum DSM 2648 is a potential probiotic that enhances intestinal barrier function. FEMS Microbiol. Lett. 309, 184-192. https://doi.org/10.1111/j.1574-6968.2010.02038.x
Attwood, G. T., A. V. Klieve, D. Ouwerkerk, and B. K. C Patel. 1998. Ammonia -hyper producing bacteria from New Zealand ruminants. Applied and Environmental Microbiology, 64:1796-1804.
Balch, W. E., Fox, G. E., Magrum, L. J., Woese, C. R. & Wolfe, R. S., 1979. Methanogens: re-evaluation of a unique biological group. Microbiol. Rev. 43, 260-96.
Bengtsson-Palme, J., Hartmann, M., Eriksson, K. M., Pal, C., Thorell, K., Larsson, D. G. J., Nilsson, R. H., 2015. METAXA2: improved identification and taxonomic classification of small and large subunit rRNA in metagenomic data. Mol. Ecol. Resour. 15, 1403-1414. https://doi.org/10.1111/1755-0998.12399
Bolger, A. M., Lohse, M., Usadel, B., 2014. Trimmomatic: a flexible trimmer for Illumina sequence data. Bioinforma. Oxf. Engl. 30, 2114-2120. https://doi.org/10.1093/bioinformatics/btu170
Buchfink, B., Xie, C., Huson, D. H., 2015. Fast and sensitive protein alignment using DIAMOND. Nat. Methods 12, 59-60. https://doi.org/10.1038/nmeth.3176
Bushnell, B., 2014. BBMap: A Fast, Accurate, Splice-Aware Aligner (No. LBNL-7065E). Lawrence Berkeley National Lab. (LBNL), Berkeley, CA (United States).
Carbonero, F., Benefiel, A. C., Gaskins, H. R., 2012. Contributions of the microbial hydrogen economy to colonic homeostasis. Nat. Rev. Gastroenterol. Hepatol. 9, 504-518. https://doi.org/10.1038/nrgastro.2012.85
Carlson, M., 2016. KEGG.db: A set of annotation maps for KEGG.
De Man, J. C., Rogosa, M., & Sharpe, M. E., 1960. A medium for the cultivation of lactobacilli. J. Appl. Bacteriol. 23(1), 130-135. https://doi.org/10.1111/j.1365-2672.1960.tb00188.x
De Filippis, F., Pasolli, E., Tett, A., Tarallo, S., Naccarati, A., De Angelis, M., Neviani, E., Cocolin, L., Gobbetti, M., Segata, N., Ercolini, D., 2019. Distinct Genetic and Functional Traits of Human Intestinal Prevotella copri Strains Are Associated with Different Habitual Diets. Cell Host Microbe 25, 444-453.e3. https://doi.org/10.1016/j.chom.2019.01.004
Dixon, P., 2003. VEGAN, a package of R functions for community ecology. J. Veg. Sci. 14, 927-930. https://doi.org/10.1111/j.1654-1103.2003.tb02228.x
Dobin, A., Davis, C. A., Schlesinger, F., Drenkow, J., Zaleski, C., Jha, S., Batut, P., Chaisson, M., Gingeras, T. R., 2013. STAR: ultrafast universal RNA-seq aligner. Bioinforma. Oxf. Engl. 29, 15-21. https://doi.org/10.1093/bioinformatics/bts635
Doyle, N., Mbandlwa, P., Kelly, W. J., Attwood, G., Li, Y., Ross, R. P., Stanton, C., Leahy, S., 2019. Use of Lactic Acid Bacteria to Reduce Methane Production in Ruminants, a Critical Review. Front. Microbiol. 10. https://doi.org/10.3389/fmicb.2019.02207
Efimov, B. A., Chaplin, A. V., Shcherbakova, V. A., Suzina, N. E., Podoprigora, I. V., Shkoporov, A. N., 2018. Prevotella rara sp. nov., isolated from human faeces. Int. J. Syst. Evol. Microbiol. 68, 3818-3825. https://doi.org/10.1099/ijsem.0.003066
Fil, J. E., Joung, S., Hayes, C. A., Dilger, R. N., 2021. Influence of rearing environment on longitudinal brain development, object recognition memory, and exploratory behaviors in the domestic pig (Sus scrofa). Front. Neurosci., 15:649536. https://doi.org/10.3389/fnins.2021.649536
Filippo, C. D., Cavalieri, D., Paola, M. D., Ramazzotti, M., Poullet, J. B., Massart, S., Collini, S., Pieraccini, G., Lionetti, P., 2010. Impact of diet in shaping gut microbiota revealed by a comparative study in children from Europe and rural Africa. Proc. Natl. Acad. Sci. 10⁷, 14691-14696. https://doi.org/10.1073/pnas.1005963107
Flint, H. J., Scott, K. P., Duncan, S. H., Louis, P., Forano, E., 2012. Microbial degradation of complex carbohydrates in the gut. Gut Microbes 3, 289-306. https://doi.org/10.4161/gmic.19897
Gaspar, C., Donders, G. G., Palmeira-de-Oliveira, R., Queiroz, J. A., Tomaz, C., Martinez-de-Oliveira, J., & Palmeira-de-Oliveira, A. 2018. Bacteriocin production of the probiotic Lactobacillus acidophilus KS400. AMB Express. 8(1), 153. doi: 10.1186/s13568-018-0679-z.
Good, M., Sodhi, C. P., Ozolek, J. A., Buck, R. H., Goehring, K. C., Thomas, D. L., Vikram, A., Bibby, K., Morowitz, M. J., Firek, B., Lu, P., Hackam, D. J., 2014. Lactobacillus rhamnosus HN001 decreases the severity of necrotizing enterocolitis in neonatal mice and preterm piglets: evidence in mice for a role of TLR9. Am. J. Physiol. Gastrointest. Liver Physiol. 306, G1021-1032. https://doi.org/10.1152/ajpgi.00452.2013
Gresse, R., Durand, F. C., Duniére, L., Blanquet-Diot, S., Forano, E., 2019. Microbiota Composition and Functional Profiling Throughout the Gastrointestinal Tract of Commercial Weaning Piglets. Microorganisms 7(9), 343. https://doi.org/10.3390/microorganisms7090343
Groschwitz, K. R., Hogan, S. P., 2009. Intestinal barrier function: molecular regulation and disease pathogenesis. J. Allergy Clin. Immunol. 124, 3-20; quiz 21-22. https://doi.org/10.1016/j.jaci.2009.05.038
Henderson G, Cox F, Ganesh S, Jonker A, Young W, Global Rumen Census Collaborators, and Janssen PH. 2015. Rumen microbial community composition varies with diet and host, but a core microbiome is found across a wide geographical range. Scientific Reports 5( )14567; doi: 10.1038/srep14567.
Hjorth, M. F., Bladel, T., Bendtsen, L. Q., Lorenzen, J. K., Holm, J. B., Kiilerich, P., Roager, H. M., Kristiansen, K., Larsen, L. H., Astrup, A., 2019. Prevotella-to-Bacteroides ratio predicts body weight and fat loss success on 24-week diets varying in macronutrient composition and dietary fiber: results from a post-hoc analysis. Int. J. Obes. 2005 43, 149-157. https://doi.org/10.1038/s41366-018-0093-2
Hoedt, E. C., 2017. Functional and comparative studies of members of the genus Methanosphaera, and their adaptations to the gut environment. PhD Thesis, The University of Queensland, Brisbane, Australia. https://doi.org/10.1007/s002849900262
Huang, L., Zhang, D., Han, W., Guo, C., 2019. High-mobility group box-1 inhibition stabilizes intestinal permeability through tight junctions in experimental acute necrotizing pancreatitis. Inflamm. Res. Off. J. Eur. Histamine Res. Soc. A1 68, 677-689. https://doi.org/10.1007/s00011-019-01251-x
Huson, D. H., Beier, S., Flade, I., Górska, A., El-Hadidi, M., Mitra, S., Ruscheweyh, H.-J., Tappu, R., 2016. MEGAN Community Edition-Interactive Exploration and Analysis of Large-Scale Microbiome Sequencing Data. PLOS Comput. Biol. 12, e1004957. https://doi.org/10.1371/journal.pcbi.1004957
Iljazovic, A., Roy, U., Gelvez, E. J. C., Lesker, T. R., Zhao, B., Gronow, A., Amend, L., Will, S. E., Hofmann, J. D., Pils, M. C., Schmidt-Hohagen, K., Neumann-Schaal, M., Strowig, T., 2020. Perturbation of the gut microbiome by Prevotella spp. enhances host susceptibility to mucosal inflammation. Mucosal Immunol. 1-12. https://doi.org/10.1038/s41385-020-0296-4
IPCC, 2014. Mitigation of Climate Change. Contribution of Working Group III to the Fifth Assessment Report of the Intergovernmental Panel on Climate Change. Cambridge University Press, Cambridge.
Jackson, R. B., Saunois, M., Bousquet, P., Canadell, J. G., Poulter, B., Stavert, A. R., Bergamaschi, P., Niwa, Y., Segers, A., Tsuruta, A., 2020. Increasing anthropogenic methane emissions arise equally from agricultural and fossil fuel sources. Environ. Res. Lett. 15, 071002. https://doi.org/10.1088/1748-9326/ab9ed2
Joblin, K. N. 1995. Methanogenic archaea. In Methods in Gut Microbial Ecology for Ruminants pp 47-53.
Johnson, J. S., Spakowicz, D. J., Hong, B.-Y., Petersen, L. M., Demkowicz, P., Chen, L., Leopold, S. R., Hanson, B. M., Agresta, H. O., Gerstein, M., Sodergren, E., Weinstock, G. M., 2019. Evaluation of 16S rRNA gene sequencing for species and strain-level microbiome analysis. Nat. Commun. 10, 5029. https://doi.org/10.1038/s41467-019-13036-1
Jorgensen, H., Theil, P. K., Knudsen, K. E. B. Enteric methane emission from pigs. Planet Earth 2011-Global Warming Challenges and Opportunities for Policy and Practice, 605-622.
Kanehisa, M., Goto, S., 2000. KEGG: kyoto encyclopedia of genes and genomes. Nucleic Acids Res. 28, 27-30. https://doi.org/10.1093/nar/28.1.27
Kawasaki, T., Kawai, T., 2014. Toll-Like Receptor Signaling Pathways. Front. Immunol. 5. https://doi.org/10.3389/fimmu.2014.00461
Kenters N, Henderson G, Jeyanathan J, Kittelmann S, Janssen PH. Isolation of previously uncultured rumen bacteria by dilution to extinction using a new liquid culture medium.J Microbiol Methods. 2011 January; 84(1):52-60.
Kovatcheva-Datchary, P., Nilsson, A., Akrami, R., Lee, Y. S., De Vadder, F., Arora, T., Hallen, A., Martens, E., Björck, I., Bäckhed, F., 2015. Dietary Fiber-Induced Improvement in Glucose Metabolism Is Associated with Increased Abundance of Prevotella. Cell Metab. 22, 971-982. https://doi.org/10.1016/j.cmet.2015.10.001
LeBlanc, J. G., Laiño, J. E., del Valle, M. J., Vannini, V., van Sinderen, D., Taranto, M. P., de Valdez, G. F., de Giori, G. S., Sesma, F., 2011. B-group vitamin production by lactic acid bacteria--current knowledge and potential applications. J. Appl. Microbiol. 111, 1297-1309. https://doi.org/10.1111/j.1365-2672.2011.05157.x
Leite, A. Z., Rodrigues, N. de C., Gonzaga, M. I., Paiolo, J. C. C., de Souza, C. A., Stefanutto, N. A. V., Omori, W. P., Pinheiro, D. G., Brisotti, J. L., Matheucci Junior, E., Mariano, V. S., de Oliveira, G. L. V., 2017. Detection of Increased Plasma Interleukin-6 Levels and Prevalence of Prevotella copri and Bacteroides vulgatus in the Feces of Type 2 Diabetes Patients. Front. Immunol. 8, 1107. https://doi.org/10.3389/fimmu.2017.01107
Li, Y., Kelly, W. J., Attwood, G. T., Reid, P. and Leahy, S. C. 2019. Methanobrevibacter boviskoreani JH1T growth on alcohols allows development of a high throughput bioassay. Proceedings of the Chicago Congress on Gastrointestinal Function Chicago 2019.
Loughman, A., Ponsonby, A.-L., O'Helv, M., Symeonides, C., Collier, F., Tang, M. L. K., Carlin, J., Ranganathan, S., Allen, K., Pezic, A., Saffery, R., Jacka, F., Harrison, L. C., Sly, P. D., Vuillermin, P., 2020. Gut microbiota composition during infancy and subsequent behavioural outcomes. EBioMedicine 52. https://doi.org/10.1016/j.ebiom.2020.102640
McCrady, M. H., 1918. Tables for Rapid Interpretation of Fermentation-tube Results. The Public Health Journal, n9(5): 201-220.
Matsuzawa-Ishimoto, Y., Hwang, S., Cadwell, K., 2018. Autophagy and Inflammation. Annu. Rev. Immunol. 36, 73-101. https://doi.org/10.1146/annurev-immunol-042617-053253
Ursula M. McCormack, Tânia Curião, Barbara U. Metzler-Zebeli, Elizabeth Magowan, Donagh P. Berry, Henry Reyer, Maria L. Prieto, Stefan G. Buzoianu, Michael Harrison, Natalie Rebeiz, Fiona Crispie, Paul D. Cotter, Orla O'Sullivan, Gillian E. Gardiner, Peadar G. Lawlor (2019). Porcine Feed Efficiency-Associated Intestinal Microbiota and Physiological Traits: Finding Consistent Cross-Locational Biomarkers for Residual Feed Intake. mSystems 4 (4):e00324-18; DOI: 10.1128/mSystems.00324-18.
Morishita, T., Tamura, N., Makino, T., Kudo, S., 1999. Production of menaquinones by lactic acid bacteria. J. Dairy Sci. 82, 1897-1903. https://doi.org/10.3168/jds.S0022-0302(99)75424-X
Mudd, A. T., Fleming, S. A., Labhart, B., Chichlowski, M., Berg, B. M., Donovan, S. M., Dilger, R. N., 2017. Dietary Sialyllactose Influences Sialic Acid Concentrations in the Prefrontal Cortex and Magnetic Resonance Imaging Measures in Corpus Callosum of Young Pigs. Nutrients 9. https://doi.org/10.3390/nu9121297
Muetzel, S., Hunt, C., and Tavendale, M. H. (2014). A fully automated incubation system for the measurement of gas production and gas composition. Animal Feed Science and Technology 196, 1-11.
Nikoletopoulou, V., Sidiropoulou, K., Kallergi, E., Dalezios, Y., Tavernarakis, N., 2017. Modulation of Autophagy by BDNF Underlies Synaptic Plasticity. Cell Metab. 26, 230-242.e5. https://doi.org/10.1016/j.cmet.2017.06.005
Overbeek, R., Begley, T., Butler, R. M., Choudhuri, J. V., Chuang, H.-Y., Cohoon, M., de Crécy-Lagard, V., Diaz, N., Disz, T., Edwards, R., Fonstein, M., Frank, E. D., Gerdes, S., Glass, E. M., Goesmann, A., Hanson, A., Iwata-Reuyl, D., Jensen, R., Jamshidi, N., Krause, L., Kubal, M., Larsen, N., Linke, B., McHardy, A. C., Meyer, F., Neuweger, H., Olsen, G., Olson, R., Osterman, A., Portnoy, V., Pusch, G. D., Rodionov, D. A., Rückert, C., Steiner, J., Stevens, R., Thiele, I., Vassieva, O., Ye, Y., Zagnitko, O., Vonstein, V., 2005. The subsystems approach to genome annotation and its use in the project to annotate 1000 genomes. Nucleic Acids Res. 33, 5691-5702. https://doi.org/10.1093/nar/gki866
Peabody, M. A., Van Rossum, T., Lo, R., Brinkman, F. S. L., 2015. Evaluation of shotgun metagenomics sequence classification methods using in silico and in vitro simulated communities. BMC Bioinformatics 16, 362. https://doi.org/10.1186/s12859-015-0788-5
Perez, R. H., Zendo, T., Sonomoto, K., 2014. Novel bacteriocins from lactic acid bacteria (LAB): various structures and applications. Microb. Cell Factories 13 Suppl 1, S3. https://doi.org/10.1186/1475-2859-13-S1-S3
Precup, G., Vodnar, D.-C., 2019. Gut Prevotella as a possible biomarker of diet and its eubiotic versus dysbiotic roles: a comprehensive literature review. Br. J. Nutr. 122, 131-140. https://doi.org/10.1017/S0007114519000680
Quast, C., Pruesse, E., Yilmaz, P., Gerken, J., Schweer, T., Yarza, P., Peplies, J., Glo\ddot{c}kner, F. O., 2013. The SILVA ribosomal RNA gene database project: improved data processing and web-based tools. Nucleic Acids Res. 41, D590-D596. https://doi.org/10.1093/nar/gks1219
Reinshagen, M., Rohm, H., Steinkamp, M., Lieb, K., Geerling, I., Von Herbay, A., Flämig, G., Eysselein, V. E., Adler, G., 2000. Protective role of neurotrophins in experimental inflammation of the rat gut. Gastroenterology 119, 368-376. https://doi.org/10.1053/gast.2000.9307
Riboulet-Bisson, E., Sturme, M. H. J., Jeffery, I. B., O'Donnell, M. M., Neville, B. A., Forde, B. M., Claesson, M. J., Harris, H., Gardiner, G. E., Casey, P. G., Lawlor, P. G., O'Toole, P. W., Ross, R. P., 2012. Effect of Lactobacillus salivarius bacteriocin Abp118 on the mouse and pig intestinal microbiota. PloS One 7, e31113. https://doi.org/10.1371/journal.pone.0031113
Robinson, M. D., McCarthy, D. J., Smyth, G. K., 2010. edgeR: a Bioconductor package for differential expression analysis of digital gene expression data. Bioinforma. Oxf. Engl. 26, 139-140. https://doi.org/10.1093/bioinformatics/btp616 Roux, V., Robert, C., Raoult, D., 2014. Non-contiguous finished genome sequence of Prevotella timonensis type strain 4401737(T.). Stand. Genomic Sci. 9, 1344-1351. https://doi.org/10.4056/sigs.5098948
Scher, J. U., Sczesnak, A., Longman, R. S., Segata, N., Ubeda, C., Bielski, C., Rostron, T., Cerundolo, V., Pamer, E. G., Abramson, S. B., Huttenhower, C., Littman, D. R., 2013. Expansion of intestinal Prevotella copri correlates with enhanced susceptibility to arthritis. eLife 2, e01202. https://doi.org/10.7554/eLife.01202
Smith, N. W., Shorten, P. R., Altermann, E. H., Roy, N. C., McNabb, W. C., 2019. Hydrogen cross-feeders of the human gastrointestinal tract. Gut Microbes 10, 270-288. https://doi.org/10.1080/19490976.2018.1546522
Smyth, G. K., 2004. Linear models and empirical bayes methods for assessing differential expression in microarray experiments. Stat. Appl. Genet. Mol. Biol. 3, Article3. https://doi.org/10.2202/1544-6115.1027
Soto-Martin, E. C., Warnke, I., Farquharson, F. M., Christodoulou, M., Horgan, G., Derrien, M., Faurie, J.-M., Flint, H. J., Duncan, S. H., Louis, P., 2020. Vitamin Biosynthesis by Human Gut Butyrate-Producing Bacteria and Cross-Feeding in Synthetic Microbial Communities. mBio 11. https://doi.org/10.1128/mBio.00886-20
Tyrrell, H. F., Reid, J. T., 1965. Prediction of the energy value of cow's milk. J. Dairy Sci. 48, 1215-1223. https://doi.org/10.3168/jds.S0022-0302(65)88430-2
Wheeler, B., Torchiano, M., 2016. ImPerm: Permutation tests for linear models.
Wong, M., Ganapathy, A. S., Suchanec, E., Laidler, L., Ma, T., Nighot, P., 2019. Intestinal epithelial tight junction barrier regulation by autophagy-related protein ATG6/beclin 1. Am. J. Physiol. Cell Physiol. 316, C753-C765. https://doi.org/10.1152/ajpcell.00246.2018
Wright, D. P., Rosendale, D. I., Robertson, A. M., 2000. Prevotella enzymes involved in mucin oligosaccharide degradation and evidence for a small operon of genes expressed during growth on mucin. FEMS Microbiol. Lett. 190, 73-79. https://doi.org/10.1111/j.1574-6968.2000.tb09265.x
Zhang, J., Kobert, K., Flouri, T., Stamatakis, A., 2014. PEAR: a fast and accurate Illumina Paired-End reAd mergeR. Bioinforma. Oxf. Engl. 30, 614-620. https://doi.org/10.1093/bioinformatics/btt593

### INDUSTRIAL APPLICABILITY

This invention relates to the use of probiotic bacteria, particularly L. rhamnosus strain FNZ129 and/or derivatives thereof, and in particular the use to improve the body weight or body condition of animals, such as a ruminant and/or monogastric animals; to improve feed efficiency, growth, productivity, and/or milk or meat yield, and/or to inhibit the growth of methane-producing bacteria and/or archaea in the digestive tract of animals; to reduce the ability of the rumen and/or gastrointestinal microbiome to produce methane, reduce methane emissions by an animal, deliver a microorganism to an animal, and/or to reduce the greenhouse gas emission footprint of an animal. Methods for using L. rhamnosus strain FNZ129 and/or derivatives thereof and feed compositions comprising the same are also provided.

1. (canceled)

2. A food or feed composition comprising Lacticaseibacillus rhamnosus (Lactobacillus rhamnosus) strain FNZ129, NMIA accession number V21/015446 dated 2 Aug. 2021, or a derivative thereof.

3. The food or feed composition of claim 2, wherein the composition is a ruminant feed composition or a feed composition for a monogastric animal.

4. The food or feed composition of claim 2, wherein the composition is a feed composition for improving the body weight and/or body composition of an animal, increasing feed efficiency in an animal, enhancing the growth and/or productivity in an animal, increasing the yield of milk and/or milk components produced from an animal, inhibiting the growth of methane-producing bacteria and/or archaea in the gastrointestinal tract of an animal, reducing the ability of the gastrointestinal microbiome to produce methane, reducing methane emissions by an animal, delivering a microorganism to an animal, and/or reducing the greenhouse gas emission footprint of an animal.

5. The feed composition of claim 4, wherein the feed composition:
a. is a ruminant feed composition, and the animal is a ruminant animal;
b. is a monogastric feed composition, and the animal is a monogastric animal
c. is a fermented yoghurt-style composition, and wherein the fermented yoghurt-style composition is formed through a process of growing L. rhamnosus FNZ129 using a milk-based carrier or non-milk-based carrier;
d. is or comprises Partial or Total Mixed Ration (TMR), corn, soybean, forage, grain, distiller's grain, sprouted grain, legumes, fibre, fodder, grass, hay, straw, silage, kernel, leaves, meal, mash feed, fruit pulp, vegetable pulp, fruit or vegetable pomace, citrus meal, wheat shorts, corn cob meal, lick block, or molasses;
e. further comprises at least one microorganism of a different species or strain, a vaccine that inhibits methanogens or methanogenesis, and/or a natural or chemically-synthesised inhibitor of methanogenesis and/or methanogen inhibitor such as bromoform; and/or
f. further comprises one or more agents selected from one or more prebiotics, one or more probiotics, one or more postbiotics, one or more sources of dietary fibre, one or more galactooligosaccharides, one or more short chain galactooligosaccharides, one or more long chain galactooligosaccharides, one or more fructooligosaccharides, inulin, one or more galactans, one or more fructans, lactulose, or any mixture of any two or more thereof.

6. (canceled)

7. (canceled)

8. (canceled)

9. (canceled)

10. The feed composition of claim 4, wherein the derivative of the L. rhamnosus FNZ129 is a metabolite of the strain, or a culture supernatant of the strain.

11. A method for:
a. improving the body weight and/or body composition of an animal,
b. increasing feed efficiency in an animal,
c. enhancing the growth and/or productivity in an animal,
d. increasing the yield of milk and/or milk components produced from an animal,
e. inhibiting the growth of methane-producing bacteria and/or archaea in the gastrointestinal tract of an animal,
f. reducing the ability of the gastrointestinal microbiome of an animal to produce methane,
g. reducing methane emissions by an animal,
h. delivering a microorganism to an animal, and/or
i. reducing the greenhouse gas emission footprint of an animal;
the method comprising the step of administering to the animal
an effective amount of Lacticaseibacillus rhamnosus (Lactobacillus rhamnosus) strain FNZ129, NMIA accession number V21/015446 dated 2 Aug. 2021, or a derivative thereof.

12. The method of claim 11, wherein the animal is a ruminant animal or a monogastric animal.

13. The method of claim 11, wherein the method inhibits the growth of methylotrophic methanogens in the forestomach and/or caecum of the animal, preferably a methanogen from the genus Methanosphaera.

14. The method of claim 11, wherein the L. rhamnosus FNZ129 or derivative thereof is administered in a composition that is a food, drink, food additive, drink additive, animal feed, animal feed additive, animal feed supplement, dietary supplement, carrier, vitamin or mineral premix, nutritional product, enteral feeding product, soluble, slurry, supplement, pharmaceutical, lick block, drench, tablet, capsule, pellet, bolus, or intra-ruminal product, or wherein the L. rhamnosus FNZ129 is encapsulated, for example in liposomes, microbubbles, microparticles or microcapsules.

15. The method of claim 14, wherein the L. rhamnosus FNZ129 or derivative thereof is administered in drinking water, milk, milk powder, milk replacement, milk fortifier, whey, whey powder, Partial or Total Mixed Ration (TMR), a feed pellet, corn, soybean, forage, grain, distiller's grain, sprouted grain, legumes, vitamins, amino acids, minerals, fibre, fodder, grass, hay, straw, silage, kernel, leaves, meal, solubles, slurries, supplements, mash feed, meal, fruit pulp, vegetable pulp, fruit or vegetable pomace, citrus meal, wheat shorts, corn cob meal, molasses, sucrose, maltodextrin, rice hulls, vermiculite, zeolites or crushed limestone.

16. The method of claim 11, wherein the method comprises administering to the animal L. rhamnosus FNZ129 in an amount of:
a. from 10⁴ to 10¹³ colony forming units per kilogram of dry weight carrier feed, optionally from 10⁸ to 10¹² colony forming units per kilogram of dry weight carrier feed;
b. from 10⁴ to 10¹⁰ colony forming units per kilogram of body weight of the animal per day, optionally from 10⁵ to 10⁸ colony forming units per kilogram of body weight of the animal per day; or
c. from 10⁴ to 10¹³ colony forming units per day, optionally from 10⁶ to 10¹³ colony forming units per day.

17. The method of claim 11, wherein the derivative of L. rhamnosus FNZ129 is a metabolite of the strain, or a culture supernatant of the strain.

18. The method of claim 11, the method comprising further administering at least one microorganism of a different species or strain, a vaccine that inhibits methanogens or methanogenesis, and/or a natural or chemically-synthesised inhibitor of methanogenesis and/or methanogen inhibitor such as bromoform.

19. The method of claim 11, wherein the L. rhamnosus FNZ129 or derivative thereof is administered separately, simultaneously or sequentially with one or more agents selected from one or more prebiotics, one or more probiotics, one or most postbiotics, one or more sources of dietary fibre, one or more galactooligosaccharides, one or more short chain galactooligosaccharides, one or more long chain galactooligosaccharides, one or more fructooligosaccharides, inulin, one or more galactans, one or more fructans, lactulose, or any mixture of any two or more thereof.

20. The method of claim 11, wherein the method:
a. enhances the growth or productivity of the animal,
b. increases the yield of milk and/or milk components produced from the animal,
c. increases the yield of milk fat, milk protein or milk solids in milk produced from the animal, and/or
d. additionally improves the body weight and/or body composition of the animal.

21. The method of claim 11, wherein the animal is:
a. a ruminant animal;
b. a bovine, goat, sheep, bison, yak, water buffalo, deer, camel, alpaca, llama, wildebeest, antelope, or nilgai;
c. a lactating ruminant animal;
d. a monogastric animal;
e. a human, pig, cat, dog, horse, donkey, rabbit, or poultry: or
f. a monogastric companion animal.

22. (canceled)

23. (canceled)

24. (canceled)

25. The method of claim 11, wherein the animal is a pre-weaning animal, for example a calf, a lamb, a piglet, or a foal; or a post-weaning animal; or wherein the L. rhamnosus FNZ129 is administered to the animal both prior to weaning and after weaning.

26. The method of claim 11, wherein the administering is to a pre-weaning animal and wherein the inhibition of the growth of methane-producing bacteria and/or archaea in the gastrointestinal tract of the animal, the reduction of methane emissions, for example methane production, by the animal, and/or the increased feed efficiency in the animal persists post-weaning.

27. The method of claim 11, wherein the inhibition of the growth of methane-producing bacteria and/or archaea in the gastrointestinal tract of the animal, the reduction of methane emissions, for example methane production, by the animal, and/or the increased feed efficiency in the animal persists for at least 2 days, 3 days, 5 days, 1 week, 2 weeks, 1 month, 2 months, 3 months, 4 months, 5 months, 6 months, 7 months 8 months, 9 months, 10 months, 11 months, 1 year, 2 years, 3 years, 4 years, 5 years, 6 years, or 7 years after the last administration of L. rhamnosus FNZ129; preferably for the life of the animal.

28. (canceled)

29. A method for producing an animal product, for example a dairy, meat, or wool product, having a reduced greenhouse gas emission footprint, the method comprising:
a. providing an animal to which the method of claim 11 has been applied, and
b. producing an animal product from the animal.

30. (canceled)

31. (canceled)

32. Lacticaseibacillus rhamnosus (Lactobacillus rhamnosus) strain FNZ129, NMIA accession number V21/015446 dated 2 Aug. 2021, or a derivative thereof, for use in improving the body weight and/or body composition of an animal, increasing feed efficiency in an animal, enhancing the growth and/or productivity in an animal, increasing the yield of milk and/or milk components produced from an animal, inhibiting the growth of methane-producing bacteria and/or archaea in the gastrointestinal tract of an animal, reducing the ability of the gastrointestinal microbiome to produce methane, reducing methane emissions by an animal, delivering a microorganism to an animal, and/or reducing the greenhouse gas emission footprint of an animal.
